(19) United States
(12) Patent Application Publication
(10) Pub. No.: US 2025/0221834 A1
(43) Pub. Date: Jul. 10, 2025

(54) SYSTEMS AND METHODS FOR TREATING VASCULAR DISEASE

(71) Applicant: Route 92 Medical, Inc., San Mateo, CA (US)

(72) Inventors: Craig Bonsignore, San Mateo, CA (US); Michi E. Garrison, San Mateo, CA (US); David Rhodes, San Mateo, CA (US); Herbert M. Mendoza, San Mateo, CA (US)

(21) Appl. No.: 19/013,852

(22) Filed: Jan. 8, 2025

Related U.S. Application Data
(60) Provisional application No. 63/619,067, filed on Jan. 9, 2024.

(51) Int. Cl. A61F 2/89; A61F 2/82; A61F 2/915; A61F 2/962

(52) U.S. Cl. CPC A61F 2/89; A61F 2/915; A61F 2/962; A61F 2002/823; A61F 2002/91575; A61F 2250/0037

(57) ABSTRACT

Disclosed are flow diverters including a self-expanding tubular member comprising a plurality of expandable cells having interconnected zig-zag rings and diagonal struts. The tubular member has a constrained configuration having a first outer diameter of at least 1.0 mm sized for delivery using a flow diverter delivery system and an expanded configuration having a second outer diameter larger than the first outer diameter. Related devices, delivery systems, and methods of using the devices and delivery systems for treating disease, particularly intracranial and cerebral aneurysms by deploying implantable expandable devices, are provided.

### CROSS-REFERENCE TO RELATED APPLICATIONS

This application claims the benefit of priority to U.S. Provisional Application Ser. No. 63/619,067, filed Jan. 9, 2024. The entire contents of the application is incorporated by reference in its entirety.

### FIELD

The present technology relates generally to medical device systems and methods, and more particularly, to medical device delivery and methods of implantation of stents, flow diverters, and other expandable implants, for the treatment of vascular disease, such as blood vessel narrowing due to vasospasm or atherosclerotic disease, intracranial stenosis or other blockages, intracranial aneurysm, and the like.

### BACKGROUND

Vascular disease caused by stenosis or narrowing of a vessel is commonly treated by endovascular implantation of scaffolding devices such as stents, often in combination with balloon angioplasty, to increase the inner diameter or cross-sectional area of the vessel lumen. Endovascular implantation of scaffolding devices, such as stents or flow diverters, can also be used to treat aneurysms in vessels of the brain (e.g., cerebral arteries) or vessels leading to the brain (e.g., intracranial arteries) to direct flow and/or assist in the implantation of a coil into the aneurysm. Flow diverters are particularly useful for treating aneurysms with wide necks that are difficult to exclude by other means, such as embolic coils. The flow diverters are targeted to be positioned starting from a distal normal segment, spanning the aneurysm, and ending in a proximal normal segment. Flow diverters are designed to have a very dense material coverage, around 30% when expanded, to exclude or limit blood flow from entering the aneurysm through the aneurysm neck. Excluding blood flow into the aneurysm reduces or eliminates the risk of aneurysm rupture due to thrombosis at the site over time.

In general, endovascular implantation of devices in cerebral and intracranial arteries and veins have been performed via smaller-sized delivery systems. Access with larger diameter systems has been challenging due, in part, to the tortuosity of the vasculature in the skull as well as the small size and delicate nature of the vessels. Navigating these arteries to deliver endovascular implants, such as flow diverters, stents, and other expandable implants, requires catheter systems having superior flexibility and deliverability, which can be challenging, especially for larger diameter catheters. Due to difficulty in navigating large-diameter delivery systems to these anatomies, flow diverters and other endovascular implants have been typically delivered through microcatheters that have an inner diameter of 0.027″ or smaller.

All currently available flow diverters are based on a braided wire design to achieve the high percentage metal coverage that achieves the desired thrombotic effect. A braid design can expand from a diameter deliverable through a 0.027″ inner diameter microcatheter to a vessel having a maximum desired vessel diameter of up to 5 mm (0.2″) while still possessing a metal coverage ratio of 30% at the expanded configuration. Examples include the Medtronic PIPELINE, the Stryker SURPASS, the Terumo FRED, and others. In contrast, stents constructed from laser-cut metal tubes, such as Nitinol, stainless steel, and other alloys are unable to accomplish the desired at least 30% metal coverage ratio due to geometric constraints.

Unfortunately, braid-style flow diverters can be difficult, time-consuming, imprecise, and risky to deliver. One problem with braid-style, self-expanding implants, such as braided flow diverters is that they may not immediately expand fully to the walls of the vessel and therefore may move during deployment, leading to time-consuming and risky maneuvers to achieve the desired wall coverage, location, and wall apposition. Significant shortening of the braided flow diverters is also a problem during deployment due to the nature of braid construction, and often leads to ineffective coverage of the aneurysm site and often requires repositioning, manipulation, or may require placement of an additional implant. Because of this, coverage of the aneurysm and/or apposition of the flow diverter against the wall is often not optimal. Poor apposition is associated with higher rates of narrowing or occlusion of the flow diverter.

Additionally, due to difficulty in navigating large diameter delivery systems to distal carotid and cerebral anatomies, devices such as flow diverters have been typically delivered through microcatheters that are 0.027″ ID or smaller. The delivery system for such devices often includes a leading distal guidewire tip, which presents risk of vessel perforation. Furthermore, braid-style implants like flow diverters terminating in wire ends often require delivery systems with additional distal-end-constraining features to enable the device to be pushed through the microcatheter. This constraining feature adds time and complexity to the deployment procedure.

There is a need for improved expandable implants, like stents and flow diverters, which can be delivered through larger-bore access systems that are able to optimally access cerebral and intracranial arteries for the treatment of diseases, such as atherosclerosis or aneurysms at these sites while providing adequate vessel coverage and improved deliverability and expansion characteristics. There is also a need for improved implant delivery systems of stents and flow diverters that are compatible with these improved expandable devices and larger-bore access devices, and which can deliver expandable implants precisely and quickly with minimal steps.

### SUMMARY

In an aspect, disclosed is a flow diverter including a self-expanding tubular member comprising a plurality of expandable cells, each of the expandable cells having interconnected zig-zag rings and diagonal struts. The tubular member has a constrained configuration having a first outer diameter of at least 1.0 mm sized for delivery using a flow diverter delivery system and an expanded configuration having a second outer diameter larger than the first outer diameter. The tubular member has a proximal end zone, a distal end zone, and a middle zone located between the proximal end zone and the distal end zone. At least the middle zone of the tubular member is laser-cut to have a material coverage of at least 25% when the tubular member is in the expanded configuration. The distal end zone and the proximal end zone can have lower material coverage than the middle zone. The proximal end zone can be a different length than the distal end zone, including longer or shorter than the distal end zone.

In an interrelated aspect, disclosed is a method of treating intracranial or cerebral aneurysm including advancing a catheter system through a base sheath towards an intracranial or cerebral vessel having a segment with an aneurysm. The catheter system includes an inner catheter having a tubular elongate body with a single lumen and a flexible, distal tapered end region; and an outer catheter having a catheter lumen and a distal end. The method includes positioning the tapered end region of the inner catheter distal to the distal end of the outer catheter; crossing the segment of vessel with the aneurysm with at least a portion of the tapered end region of the inner catheter; advancing the outer catheter over the inner catheter and positioning a distal end region of the outer catheter across the aneurysm; withdrawing the inner catheter from the catheter lumen and maintaining the outer catheter in place across the aneurysm; advancing a flow diverter delivery system having a flow diverter through the catheter lumen to the distal end region of the outer catheter; withdrawing the outer catheter while maintaining the flow diverter delivery system in place; and deploying the flow diverter across the segment with the aneurysm. The proximal zone can be a different length than the distal zone, including longer or shorter. The method can further include deploying the low-density proximal zone across a side branch.

In an interrelated aspect, disclosed is a flow diverter having a self-expanding tubular member with a plurality of expandable cells, each of the expandable cells including interconnected zig-zag rings and diagonal struts. The tubular member has a constrained configuration having a first outer diameter sized for delivery using a flow diverter delivery system and an expanded configuration having a second outer diameter larger than the first outer diameter. The tubular member has a proximal end zone near a proximal end, a distal end zone near a distal end, and a middle zone located between the proximal end zone and the distal end zone. At least the middle zone of the tubular member is laser-cut to have a higher material coverage when the tubular member is in the expanded configuration compared to the proximal end zone or the distal end zone.

In an interrelated aspect, disclosed is an implant delivery system including an inner core member with an elongate shaft having a single inner lumen, a recessed section, and a tip distal to the recessed section; an outer tubular member having a length sufficient to extend over the recessed section and that is retractable relative to the inner core member to expose the recessed section; and an expandable device. When the expandable device is assembled for delivery by the delivery system, the expandable device is mounted around the recessed section of the inner tubular member and the distal end of the outer tubular member is positioned distal to the expandable device to constrain the expandable device within the recessed section, and the tip projects distal to the distal end of the outer tubular member. The tip has a flexibility, a shape, a taper length, and a taper angle configured for atraumatic delivery of the delivery system to a vessel in the brain with or without a guidewire.

In an interrelated aspect, disclosed is system for treating an intracranial vessel including an access catheter having a lumen extending from a proximal opening to a distal opening at a distal end of the access catheter; and an implant delivery system sized to be received within the lumen of the access catheter and having no separate restraining sleeve. The implant delivery system includes an inner core member with an elongate shaft having a single inner lumen, a recessed section, and a tip distal to the recessed section; and an expandable device mounted around the recessed section. The access catheter of the system functions as a restraining sleeve for the expandable device of the implant delivery system.

In an interrelated aspect, disclosed is an implant delivery system including an inner core member with an elongate shaft having a single inner lumen, a recessed section having at least one grip segment, and a tip distal to the recessed section. The delivery system includes an outer tubular member having a length sufficient to extend over the recessed section and that is retractable relative to the inner core member to expose the recessed section; and an expandable device. When the expandable device is assembled for delivery by the delivery system, the expandable device is mounted around the recessed section of the inner tubular member such that regions of the expandable device engage with the at least one grip segment, and the distal end of the outer tubular member is positioned distal to the expandable device to constrain the expandable device within the recessed section, and the tip projects distal to the distal end of the outer tubular member, the tip having a flexibility, a shape, a taper length, and a taper angle configured for atraumatic delivery of the delivery system to a vessel in the brain with or without a guidewire. The expandable device can be a flow diverter having an open cell design or a closed cell design. The flow diverter can be a laser-cut expandable metal tube having a dense material coverage region. The at least one grip segment can include a plurality of discreet grip segments and the expandable device is an open cell design flow diverter.

### BRIEF DESCRIPTION OF THE DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity.

FIG. 1A is a cross-sectional view of an implementation of a cut-tube flow diverter in the collapsed delivery configuration;

FIG. 1B is the flow diverter of FIG. 1A in the “as cut” configuration showing a flat (unrolled) view of the device pattern;

FIG. 1C is a detail view of the end feature of device in FIG. 1B;

FIG. 1D is a detail view of the end feature of device in FIG. 1C with assembled radiopaque marker;

FIG. 2A is a cross-sectional view of an interrelated implementation of a cut-tube flow diverter with an asymmetrical pattern in the collapsed delivery configuration;

FIG. 2B is the flow diverter of FIG. 2A in the “as cut” configuration showing a flat (unrolled) view of the device pattern;

FIG. 3 is an interrelated implementation of a cut-tube flow diverter with flared ends;

FIG. 4A shows components of an implant and implant delivery system;

FIG. 4B shows the implant and implant delivery system of FIG. 4A assembled in a delivery configuration;

FIG. 4C shows the implant and implant delivery system of FIG. 4B with the implant partially deployed;

FIG. 5A shows components of an interrelated implementation of an implant and implant delivery system with a locking mechanism;

FIG. 5B shows a partial view of the implant and implant delivery system of FIG. 5A assembled in a delivery configuration;

FIG. 5C shows the implant and implant delivery system of FIG. 5B with the implant partially deployed;

FIG. 6A shows details of the locking mechanism of the implant to the delivery system of FIG. 5A;

FIG. 6B shows details of the locking mechanism of FIG. 6A in the assembled configuration;

FIG. 6C shows an isometric view of the locking mechanism of FIG. 6A in the assembled configuration;

FIG. 7A shows an access catheter system for accessing a treatment site in a vessel;

FIG. 7B shows the access catheter system of FIG. 7A assembled for use;

FIG. 8A is a detail view of a distal end region of a catheter advancement element taken along circle C-C of FIG. 7A;

FIG. 8B is a detail view of a distal end region of a catheter advancing element having a guidewire positioned in the inner lumen of the catheter advancement element of FIG. 8A;

FIG. 9A shows an assembled catheter system accessing an intracranial treatment site, with a base sheath positioned in the internal carotid artery (ICA), an outer catheter advanced in the distal ICA, and an inner catheter crossing the vessel in the area of the treatment site A;

FIG. 9B shows the outer catheter of FIG. 9A advanced across the treatment site and the inner catheter withdrawn;

FIG. 9C shows an implant delivery system advanced across the treatment site and the outer catheter withdrawn;

FIG. 9D shows the implant restraining sleeve withdrawn and an implant deployed across a treatment site;

FIG. 9E shows an asymmetric implant of FIGS. 2A and 2D, deployed across a treatment site and side branch with the implant delivery system withdrawn;

FIGS. 10A and 10B are variations of the flow diverter of FIGS. 1A-1B in the “as cut” configuration showing a flat (unrolled) view of the device pattern;

FIG. 11A is an alternate embodiment of a flow diverter, in the “as cut” configuration showing a flat (unrolled) view of the device pattern;

FIG. 11B is a detail of the “as cut” configuration of FIG. 11A taken at box B;

FIGS. 11C and 11D are details of the central section of the flow diverter of FIG. 11A, in the expanded and collapsed delivery configuration, respectively;

FIG. 12 shows an interrelated implementation of the inner core member of an implant delivery system;

FIG. 13A is a detailed view of the distal end region of the inner core member of FIG. 12;

FIG. 13B is a detailed view of an interrelated implementation of inner core member of FIG. 12;

FIG. 14 is a cross-sectional view of a grip portion of an inner core member of FIG. 13B;

FIG. 15 is a cross-sectional view of a distal end region of another implementation of an implant delivery system.

It should be appreciated that the drawings are for example only and are not meant to be to scale. The drawings are intended to be illustrative to dimensions including metal coverage percentages. The drawings are not to scale in absolute terms or comparatively. It is to be understood that devices described herein may include features not necessarily depicted in each figure.

### DETAILED DESCRIPTION

Described herein are expandable implants, such as flow diverters, coils, and stents, and implant delivery systems and methods that are compatible with large-bore access systems. The devices, systems and methods take advantage of the large-bore access to provide improvements over existing expandable implants, such as braided flow diverters and associated microcatheter-based delivery systems to enable more precise, safe, and rapid treatment of vessel diseases, such as aneurysms, vasospasm, intracranial atherosclerotic disease (ICAD), intracranial stenosis, and the like. These devices and systems can be delivered through any large-bore neurovascular access systems. Also described are improved large-bore access systems that facilitate the speed, safety, and ease of accessing intracranial and cerebral arteries to implant expandable devices, such as stents or flow diverters at their intended site, even despite navigational challenges.

Where the phrase “access catheter” is used herein, such a catheter may be used for other purposes besides or in addition to access, such as the delivery of fluids to a treatment site or as an aspiration catheter. Alternatively, the access systems described herein may also be useful for access to other parts of the body outside the vasculature. Similarly, where the phrase “implant,” “expandable implant,” or “working device” is described as being an expandable implant, cerebral treatment device, stent, coil, or flow diverter, other interventional devices can be delivered using the access and delivery systems described herein. Preferably, the working device is an implantable structure-temporary and retrievable or an implant that remains in place following a procedure. The working device can be a stent. As used herein, the term “stent” refers to a working device that is designed for use within a bodily structure such as within a body lumen and that is capable of undergoing a shape change from a lower profile insertion configuration to a higher profile deployed configuration. A stent refers to both balloon-expandable and self-expanding stents. A stent may be uncovered or covered with a material such as with a mesh, fabric sleeve, or graft material. A stent may be coated with a material such as a polymer or one or more drugs. A stent refers to an implant that remains in place within the bodily structure for a period of time following a procedure to continue providing a therapeutic effect. A stent may be permanent such as a metal stent or semi-permanent such as a bioabsorbable stent that erodes or is absorbed in a given time-frame. The stent may be a braided design, a cut metal tube design, or a multi-layer or compound design with more than one expandable element such as a braid and/or cut tube elements coupled together to form a single implant device. Stent may be used interchangeably with other terms describing expandable implants, such as flow diverters.

While some implementations are described herein with specific regard to accessing a neurovascular anatomy or delivery of an expandable cerebral or intracranial treatment device, the systems and methods described herein should not be limited to this and may also be applicable to other uses. For example, the catheter systems described herein may be used to deliver working devices to an extracranial vessel including the carotid vessels leading to the cerebral anatomy, or a target vessel of a coronary anatomy, peripheral anatomy, or other vascular anatomy. Coronary vessels are considered herein including left and right coronary arteries, posterior descending artery, right marginal artery, left anterior descending artery, left circumflex artery, M1 and M2 left marginal arteries, and D1 and D2 diagonal branches. Any of a variety of peripheral vessels are considered herein. Any of a variety of venous targets are also considered herein including coronary and peripheral veins and intracranial veins and sinuses, such as the dural venous sinuses.

As used herein, “embolus” or “embolus material” or “embolic material” or “embolic region” refers to material within a zone of an occlusion site that is denser or a relatively hard consistency that is preferably placed in contact with a distal end of an aspiration catheter to successfully perform aspiration embolectomy. The embolus may be a thrombus (a clot of blood) or other material that formed at a first blood vessel location (e.g., a coronary vessel), breaks loose, and travels through the circulation to a second blood vessel location. As used herein, “in situ thrombus” or “thrombus material” or “thrombotic material” or “thrombotic region” or “in situ clot material” or “clot material” refers to material within a zone of an occlusion site that accumulates in situ at the site of the embolus and is often less dense or relatively soft and fluid-like. As used herein, “organized thrombus” refers to in situ thrombus material or clot material that accumulates at the site of embolus and is denser and less fluid-like than the in situ clot material.

As used herein, “an occlusion” or “an occlusion site” or “occlusive material” refers to the blockage that occurred as a result of an atherosclerotic lesion or embolus lodging within a vessel and disrupting blood flow through the vessel or a stenosis within a vessel or sinus. The occlusion or occlusive material can include both thrombus and embolus as well as another non-thrombotic narrowing of the vessel.

As used herein, “an aneurysm” refers to the ballooning out of a weakened section of vessel wall. A “cerebral aneurysm” or “intracranial aneurysm” refers to an aneurysm in a vessel of the brain.

While some implementations are described herein with specific regard to accessing a neurovascular anatomy for application of aspiration or delivery of a flow diverter, the systems and methods described herein should not be limited to this and may also be applicable to other uses such as the delivery of a stent for treatment of atherosclerosis or other narrowing or blockage or embolic coil-supporting scaffold. For example, the catheter systems described herein may be used to deliver working devices to the carotid artery or intracranial artery or a vein. Where the phrase “distal access catheter” or “aspiration catheter” is used herein that the catheter can be used for aspiration, the delivery of fluids to a treatment site or as a support catheter, or distal access providing a conduit that facilitates and guides the delivery or exchange of other devices such as a guidewire or expandable interventional devices such as stents or stent retrievers, coils, or flow diverters.

### Expandable Implants

Disclosed herein are expandable implants, including flow diverters, that greatly improve the deployment and performance compared to current braided-style flow diverters. The flow diverters can be self-expanding, cut-tube style implants that unlike a braided wire tube, expand to full diameter much more quickly and accurately. There is no need to constrain or cover the distal end of the implant because the cut-tube construction lacks wire ends like some braided implants do. The cut-tube flow diverters described herein also do not experience significant foreshortening when deployed, as braided wire scaffolds do. The cut pattern can be designed to achieve wall apposition and coverage sufficient to achieve flow diversion, prevention, or rejection of blood flow into the aneurysm, and/or isolation of an aneurysm, with a single layer of material (as opposed to a braid, with a crossed wire surface), resulting in a smoother and less thrombogenic inner surface. Finally, a cut-tube flow diverter has the capability of a change in design, such as feature density over the length of the device, to enable benefits such as anchoring ends without blocking side branches to blood flow.

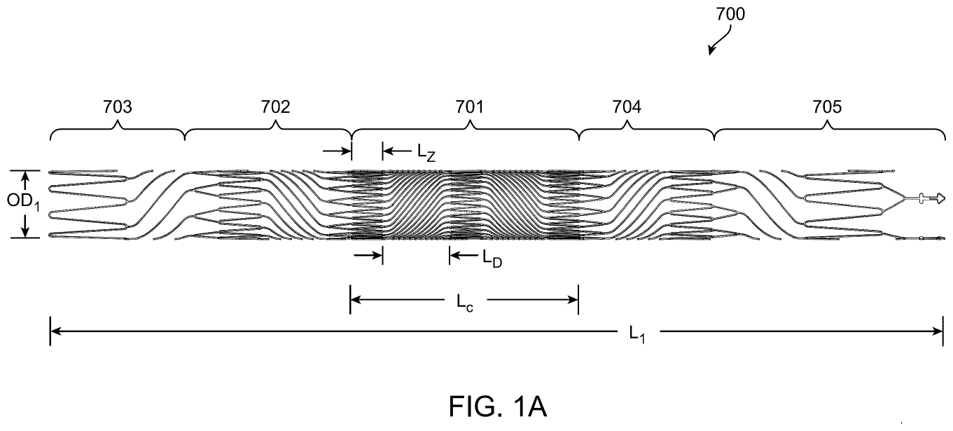

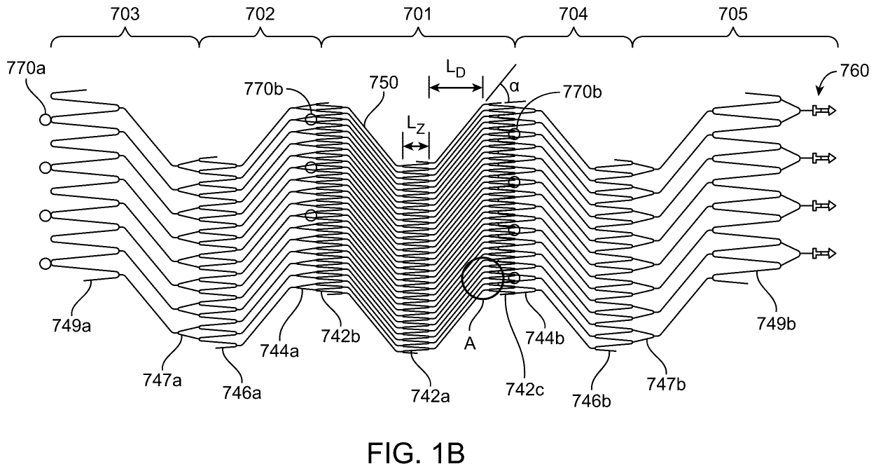

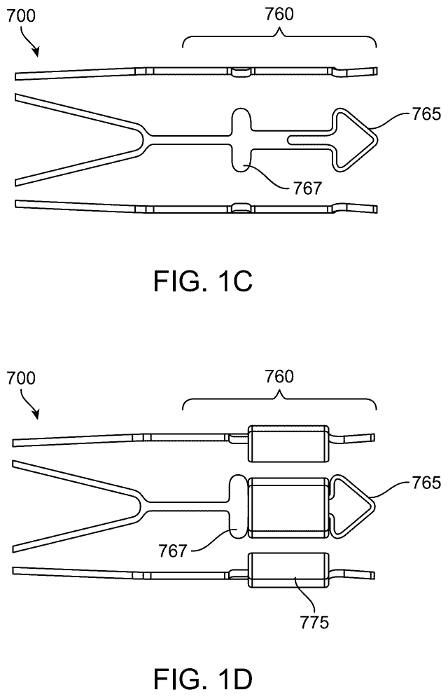

FIGS. 1A-1D show implementations of cut-tube-style flow diverter 700. In each implementation, the flow diverter 700 is a generally tubular member or element that is non-braided and having an open distal end and an open proximal end, the distal end being further away from the user during advancement through a vessel and the proximal end being closer to the user during advancement through the vessel. A longitudinal axis extends between the distal and proximal ends. FIG. 1A shows the flow diverter 700 in cross-section in a constrained configuration having a first outer diameter OD1 and first length L1. The first outer diameter OD1 accommodates insertion of the flow diverter 700 into and navigation through the vasculature to the treatment site. Upon reaching the treatment site, the flow diverter 700 is deployed and expands to a second outer diameter OD2 and second length L2. Thus, OD2 is larger than OD1.

The constrained outer diameter OD1 of the flow diverter 700 can be about 0.89 mm (0.035″) to about 3 mm (0.118″), preferably about 1.5 mm (0.06″) to 2.5 mm (0.10″), or about 1.60 mm. This constrained outer diameter OD1 is relatively large compared to the constrained outer diameter of a conventional braided-style flow diverters. This is enabled by the larger-bore access system described herein that is configured to reach distal sites for deployment of the flow diverter. The deliverability of the large-bore access system, which will be described in more detail below, is capable of delivering the larger constrained outer diameter OD1 flow diverter, also enables the flow diverter to be designed with greater material coverage when expanded.

The expanded outer diameter OD2 of the flow diverter 700 can be about 2 mm (0.08″) up to about 6 mm (0.24″), preferably between about 2.5 mm (0.10″) and about 5 mm (0.2″), depending on the anatomic requirements. The flow diverter 700, when expanded, is preferably suitable for vessels up to 5 mm in diameter. The flow diverter length L1 can also be manufactured depending on the anatomic requirements. For example, the length L1 of the flow diverter prior to expansion can vary from 10 mm to 50 mm including any dimension between and preferably from 10 mm to 35 mm.

The length L2 of the flow diverter after expansion can be in the same range. The cut-tube-style flow diverters 700 described herein undergo a minimal amount of foreshortening upon deployment from the constrained state to the expanded state in the vessel. This provides an advantage over braided-wire style flow diverters that significantly foreshorten upon deployment. The flow diverter designs shown in FIGS. 1A-1B shorten less than 10%, less than 5%, or about 1% or less when expanded from about 1.6 mm to about 4.0 mm. For example, the constrained length L1 can be about 20.7 mm and the expanded length L2 can be about 20.5 mm, which is less than about 1% foreshortening. In contrast, a braided stent shortens by about 50% when expanded to 4 mm (see Instructions For Use of the Surpass Evolve Flow Diverter System; Stryker Neurovascular). This extreme foreshortening of braided flow diverters requires precision placement of the distal end to ensure full coverage of the target segment. The distal end must extend from a site distal to the aneurysm in a normal vessel segment so that the middle section of the flow diverter can be positioned across the aneurysm neck and to a normal vessel on the proximal side of the aneurysm. Extreme foreshortening of a flow diverter can lead to “missed” deployments especially in curved or tortuous anatomy. If the aneurysm neck is very large or if the vessel is tapered at the aneurysm neck, the final length and position is even more unpredictable.

The flow diverter 700, when the tubular member is in the expanded outer diameter, is capable of achieving a material coverage suitable for treating aneurysms. The material coverage (also referred to herein as material density) can vary, but is generally between about 25%-35% material coverage (+/−5%). “Material coverage” as used herein means the surface area of the outer surface of the flow diverter divided by the surface area of the inner lumen a cylindrical tube in which the flow diverter fits in the expanded configuration. The material of the flow diverter provides the material coverage, which is the inverse of porosity of the flow diverter, which is a function of the amount of open space of the flow diverter upon expansion.

FIG. 1B is an unrolled view showing the flat pattern of the flow diverter of FIG. 1A. The flow diverter 700 includes a series of circumferential rings 742 which are formed in a zig-zag pattern. The rings 742 are connected by struts 750, with each zig-zag peak of circumferential ring 742 connected to a peak of adjacent circumferential ring 742 by a strut 750. The connections are offset such that struts 750 are diagonal with respect to the axis of the flow diverter 700. The height and number of zig-zags of circumferential rings 742 as well as the length and angle α of the diagonal struts 750 relative to the longitudinal axis A of the flow diverter are selected to provide a targeted material coverage of at least 25% or at least 30% when expanded and still be able to collapse down to a diameter of about 0.070″ or less, whilst providing a collapsed flexibility that will allow it to be navigated through tortuous anatomy to reach a targeted intracranial arterial site. This pattern with each peak connected falls in the category of “closed cell”, which provides the benefit of allowing the flow diverter to be re-sheathed and re-deployed during placement, if required by the procedure.

The flow diverter 700 can be designed to have a consistent pattern along its length or can vary in pattern over its length. Still further, the flow diverter 700 can have the same pattern of cells along its entire length, but the density of the pattern changes depending on the needs of the anatomy. The differences in pattern along the length of the flow diverter 700 can be selected to modify the strength and percent material coverage for optimal performance. The variation in pattern can form different zones along the length of the flow diverter. Specifically, the high-density zone 701 of the flow diverter 700 for flow diversion can have a maximum material density to divert flow away from anatomy external to the zone, such as an aneurysm. The flow diverter 700 can have intermediate-density zones 702, 704 on either side of the high-density zone 701. The intermediate-density zones 702, 704 can have a material density or material coverage that is lower than the high-density zone 701. The flow diverter 700 can have two end zones 703, 705 on either side, respectively, of the intermediate-density zones 702, 704, with yet lower material density than intermediate-density zones 702 and 704. At least the middle zone of the tubular member is laser-cut to have a material coverage of at least about 25% when the tubular member is in the expanded configuration. The middle zone can have different properties from one or both of the proximal end zone and the distal end zone. The middle zone can have greater material coverage than one or both of the proximal end zone and the distal end zone. One or both of the proximal end zone and the distal end zone can be laser-cut to have a material coverage that is less than 25% or less than 15% compared to the material coverage of the middle zone, which is at least 25|%. One or both of the proximal end zone and the distal end zone can include a different construction compared to the middle zone, such as a braided or woven construction.

FIGS. 1A-1B show a flow diverter 700 with sections of varying density. For example, flow diverter 700 has a section of high-density zone 701 with one or more zig-zag rings 742a in the pattern (one ring is shown but more than one ring is possible, depending on the desired length of the high-density section) and zig-zag rings 742b and 742c on the distal and proximal ends of high-density zone 701, respectively. Each ring of zig-zags is connected to the adjacent ring with diagonal struts 750. This high-density zone 701 is positioned across the entirety of the aneurysm and provides the flow diverting function of the device 700. Sections of reduced density, referred to herein as intermediate-density zones 702 and 704, are positioned on the distal and proximal side of high-density zone 701, respectively, and sections of yet further reduced density 703 and 705 on the distal and proximal side of intermediate-density zones 702 and 704, respectively.

The pattern can be modified at the ends, to allow for less dense ends, as in sections 702 and 704 of FIG. 1A, and/or terminate in even less dense ends 703 and 705, also of FIG. 1A. In an embodiment, shown in FIG. 1A, the reduced density pattern of the intermediate-density zones 702 and 704 is achieved by adding a row of V-struts to the outer edges of zig-zag rings 742b and 742c. For example, V-struts 744a are adjacent to zig-zag ring 742b, with the leg of each V-strut connected to two adjacent zig-zag peaks of ring 742b such that the number of peaks of V-struts 744a is half the number of peaks of zig-zag ring 742b. A similar pattern is made on the V-struts 744b attached to zig-zag ring 742c.

Addition reduction in density pattern can be achieved by adding another row of V-struts to zig-zag pattern further towards the end of the stent. For example, V-struts 747a are attached to zig-zag ring 746a such that the number of V-strut peaks of V-struts 747a is half the number of peaks of zig-zag rings 746a.

This section is terminated on the distal end with zig-zag ring 749a. The amplitude of ring 749a may be the same as that of V-struts 747a, or, as shown, may be higher. This may be desirable to increase the anchoring force of the distal end, and/or to allow the end to be flared as described later.

A similar pattern can occur on the proximal half of flow diverter 700, with zig-zag rings 742c and V-struts 744b, and with zig-zag ring 746b and V-struts 747b, and with proximal-most zig-zag ring 749b.

In an example, zig-zag rings 742a, 742b, and 742c have 32 peaks counted at one end of the ring (or 64 peaks counting both proximal and distal peaks) around the circumference, V-struts 744a and 744b and zig-zag rings 746a and 746b have 16 peaks, V-struts 747a and 747b and zig-zag rings 749a and 749b have 8 peaks. This ratio of 4:2:1 peaks can remain the same while the actual number of peaks can vary, depending on the OD and/or crimped profile requirements of the flow diverter 700. In another example, flow diverter has a high-density zone 701 but only one lower density distal section and one lower density proximal section.

The diagonal connecting struts 750 can be curved at each end, such that the connection to the peaks of zig-zags and V-struts are parallel or close to parallel to the longitudinal axis of the flow diverter, the longitudinal axis parallel to a lumen extending through the tubular body, but the mid-section is at an angle α to the longitudinal axis. The length and angle of the connecting struts can vary, as required by the expansion characteristics and device flexibility requirements of flow diverter 700.

In one example, the diagonal connecting struts 750 are connected at a first end to a peak in one zig-zag ring and at a second end are connected to a peak of the adjacent zig-zag ring, which is rotated about 8 peaks, about 6 peaks or at least 4 peaks circumferentially from the connection of the first end. Moving longitudinally down the flow diverter, the diagonal connecting struts 750 in each are angled in the opposite direction. A longitudinal length of the connecting strut sections is longer in the middle zone than a longitudinal length of the zig-zag rings 742a, 742b, 742c by about a ratio of 2:1, or at least about 50% greater. The longitudinal length of the diagonal connecting struts 750 remains substantially the same between the middle zone, proximal zone and distal zone while the longitudinal length of the zig-zag rings increases from the middle zone to the proximal and distal zones.

The length of the high-density zone 701, intermediate-density zones 702 and 704, and low-density zones 703 and 705 can vary. As seen in FIG. 1A, the high-density zone 701 can have a length Le designed to be longer or shorter to match the needs of the anatomy being treated. As an example, a flow diverter having an overall length L2 of about 20 mm, can incorporate a high-density zone 701 having a length L_{c} that is between 10 mm and 20 mm. The flow diverter can have a variety of lengths L2, expanded diameters OD2, and middle zone lengths L_{c}.

The lengths and number of the zig-zag rings 742 and sections of the diagonal struts 750 may also vary. For example, as seen in FIG. 1B, the high-density zone 701 of the flow diverter 700 has three zig-zag rings (i.e., 742a, 742b, 742c), each with length L_{Z} and two sections of diagonal struts 750, each section with length L_{D} and Angle α relative to the longitudinal axis of the flow diverter 700. The length L_{D} and Angle α of the sections of diagonal struts 750 dictate how much each diagonal section winds around the circumference of the flow diverter 700. FIG. 1A shows each section of diagonal struts 750 winds about ¼ the diameter of the circumference of the flow diverter 700. The lengths L_{Z} and L_{D} may remain constant along the length of flow diverter 700, or they may vary. The number of diagonal strut sections may also vary, to adjust the overall length of high-density zone 701.

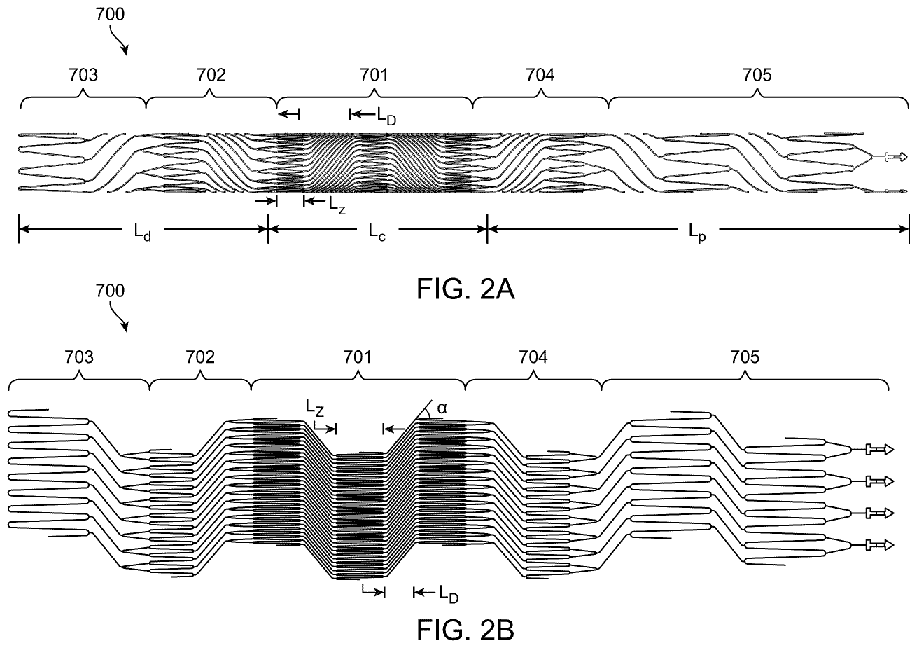

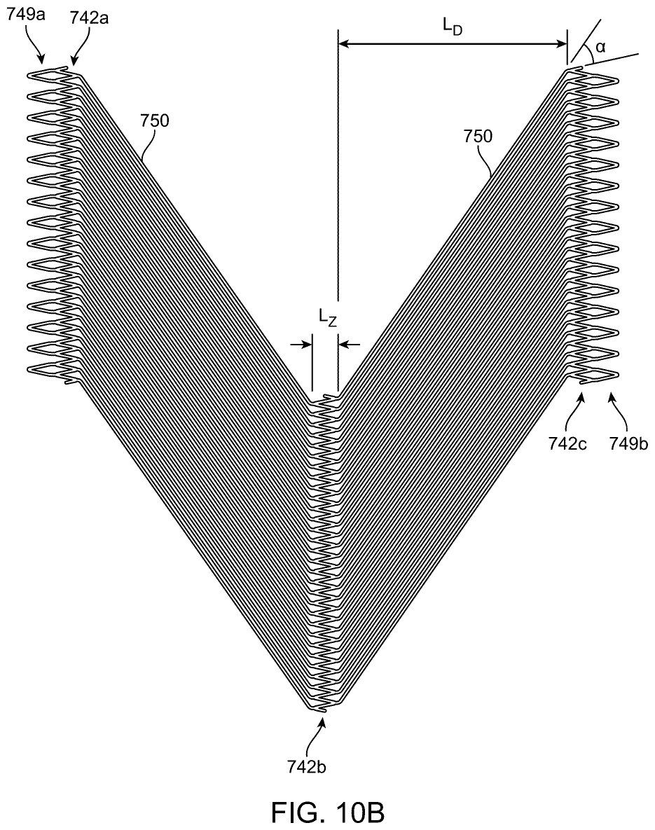

The lengths L_{Z} and L_{D} and angle α dictate the characteristics of the deployed flow diverter 700, for example, the amount of material coverage across the aneurysm neck, the radial strength, and the apposition of the device against the vessel wall, particularly in a curved segment of a blood vessel. In some implementations, a flow diverter 700 with 32 apices, L_{Z} can range from 1 mm to 3 mm, and L_{D} can range from 2 mm to 20 mm or more. The flow diverter 700 of FIGS. 2A-2B incorporates longer zig-zag rings 742 where L_{Z} is almost equal to L_{D}. Long zig-zag rings 742 provide more radial strength, but poorer apposition in curved segments. Additional variations are shown in FIGS. 10A and 10B. In FIG. 10A, there are three rows of diagonal struts 750, where L_{Z} is about 0.8 mm and L_{D} is about 7.2 mm, and Angle α (as measured in the flat configuration with respect to the longitudinal axis of the flow diverter 700) is 55 degrees, resulting in diagonal struts 750 winding around the circumference about 210 degrees when expanded and 640 degrees when constrained. In FIG. 10B, there are two rows of diagonal struts 750, where L_{Z} is the same length of 0.8 mm, but each row of diagonal struts 750 has a longer length L_{D} of about 9.4 mm and Angle α is 55 degrees, resulting in diagonal struts 750 winding around the circumference about 300 degrees when expanded and 840 degrees when constrained. The shorter length Ly in the embodiments of FIGS. 10A and 10B as compared to the embodiments of FIGS. 1A-1B and FIGS. 2A-2B result in a flow diverter that has better apposition in a curved segment. The flow diverter may also have just one row of long diagonal struts 750 with no connecting zig-zag rings 742, resulting in diagonal struts 750 winding around the entire circumference or more. In all variations, the number of rows and length and angle of diagonal struts 750 can be modified to provide longer or shorter coverage distance as desired by a user.

In an interrelated implementation, the flow diverter 700 may have a different cut pattern at either end to optimize the deployment characteristics resulting in an overall asymmetric design. FIG. 2A is a cross-sectional view and FIG. 2B is the flow diverter of FIG. 2A in the “as cut” configuration showing a flat (unrolled) view of the device pattern. The figures show an example of an asymmetrical flow diverter 700, in which zones 702 and 703 are shorter than zones 704 and 705. In this example, flow diverter 700 has a high-density zone 701 with length Lc, intermediate-density zones 702 and 703 with overall length Ld and lower density proximal sections 704 and 705 with overall length Lp, where Ld is shorter than Lp. This asymmetric design may be preferable if the aneurysm is near a major side branch or perforator, and it is desired to anchor the proximal end of the flow diverter across the perforator or side branch vessel without significantly blocking flow, while the high-density zone 701 is across the aneurysm itself. In the case of a side branch, the low-density zone 705 of the device can allow anchoring across the side branch without blocking subsequent access of devices into the side branch. Alternately, the length Ld of distal ends, including zones 702 and 703, may be longer than the length Lp of proximal ends, including zones 704 and 705, to prevent access and allow flow through a perforator side branch while anchoring across the side vessel distal to the aneurysm being flow diverted.

The flow diverters 700 in FIGS. 10A and 10B have a high-density zone formed by three regions of diagonal struts 750 or two regions of diagonal struts 750, respectively. The distal region of diagonal struts 750 (i.e., the region nearest a distal part of the flow diverter 700) can connect to a distal zig-zag ring 742a and the proximal-most region of diagonal struts 750 (i.e., the region nearest a proximal part of the flow diverter 700) can connect to a proximal zig-zag ring, or 742c in FIG. 10B or 742d in FIG. 10A. The distal-most end of the flow diverter 700 can incorporate a terminal zig-zag ring 749a distal to distal-most zig-zag ring 742a and the proximal-most end of the flow diverter 700 can incorporate a terminal zig-zag ring proximal to the proximal-most zig-zag ring. In the example of the flow diverter 700 of FIG. 10A having three regions of diagonal struts 750, the proximal-most zig-zag ring 742d has terminal zig-zag ring 749d located proximal to it. In the example of the flow diverter 700 of FIG. 10B having two regions of diagonal struts 750, the proximal-most zig-zag ring 742c has terminal zig-zag ring 749c located proximal to it. The amplitude of terminal zig-zag rings 749a, 749b can be the same as or higher than that of the zig-zag rings adjacent to the terminal rings.

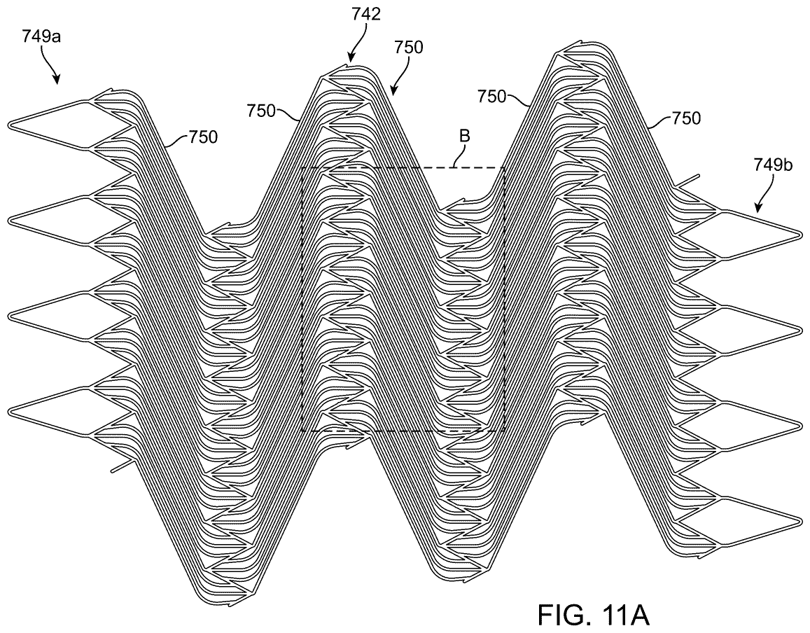

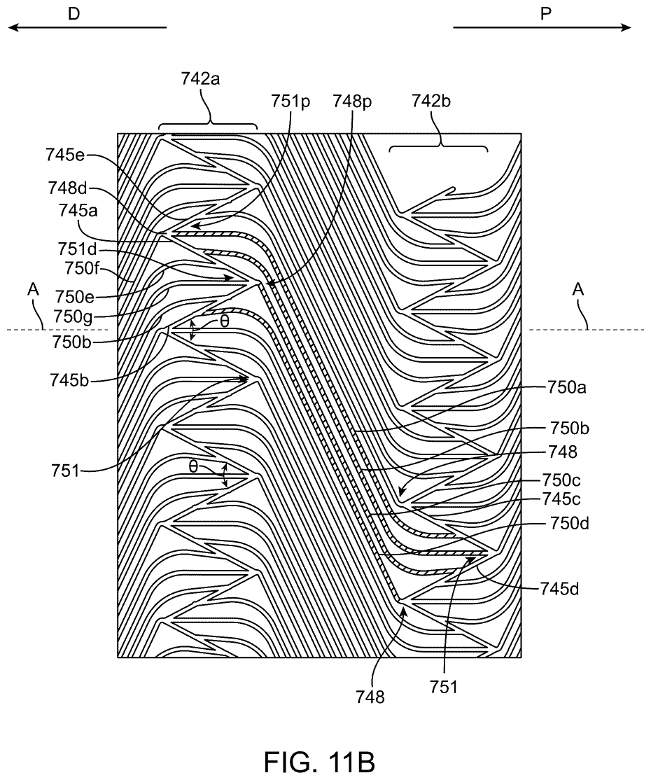

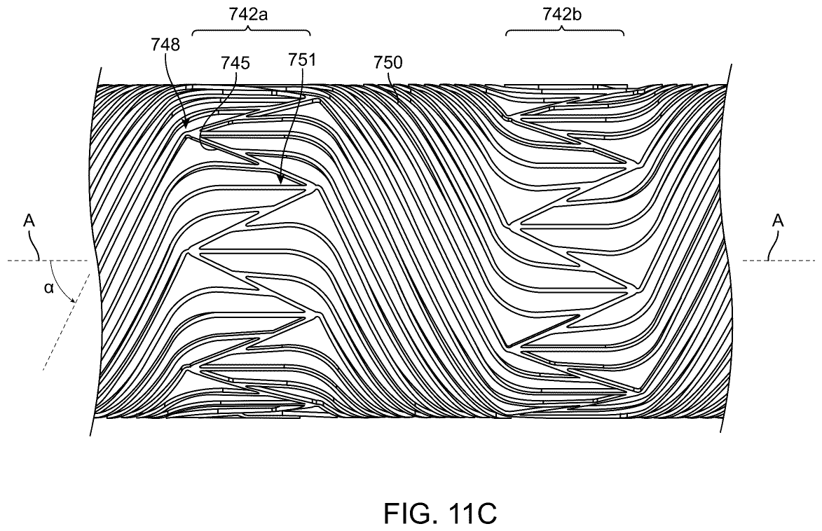

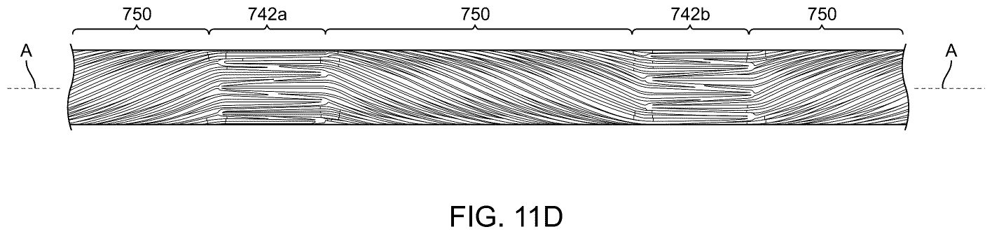

In another interrelated implementation of a flow diverter 700, as shown in FIGS. 11A-11D, the flow diverter 700 may have multiple diagonal struts 750 (e.g., two or more) connecting one “V” formed by a pair of adjacent angled struts 745a, 745b of a first zig-zag ring 742a to one “V” formed by another pair of adjacent angled struts 745c, 745d of a second zig-zag ring 742b. These multiple struts can increase the uniformity of the gap distances in the high-density (flow diversion) zone 701. FIG. 11A is an “as cut” configuration showing a flat (unrolled) view of the device pattern and FIG. 11B is a detail view of FIG. 11A taken at box B. Like the flow diverter 700 of FIGS. 1A-1B, the flow diverter 700 of FIGS. 11A-11B incorporates a series of circumferential zig-zag rings 742 connected by generally diagonal struts 750, connecting one ring 742a to an adjacent ring 742b in a generally helical pathway. As discussed above, with respect to the flow diverter of FIGS. 1A-1B and 2A-2B, the sections of connecting diagonal struts 750 are alternatingly clockwise and counterclockwise in curve direction, so as not to create an overall twisted pattern. However, as seen most clearly in flat view FIG. 11A and detail view FIG. 11B, the flow diverter 700 has multiple diagonal struts 750 connecting an angled strut 745a of a “V” formed by a pair of adjacent angled struts 745 of one adjacent ring 742a to an angled strut 745c of a “V” formed by a pair of adjacent angled struts 745 of the adjacent ring 742b. All diagonal struts 750 within one section of diagonal struts are arranged generally parallel to one another along at least a portion of the length of the diagonal struts and generally in a helical path. However, end regions of the diagonal struts 750 within one section of diagonal struts may be curved or incorporate a curve, for example, near the connection point between the diagonal strut 750 and its respective angled strut 745 of the zig-zag ring 742 such that the diagonal strut 750 becomes generally parallel to the longitudinal axis within the interior of the “V” formed by a pair of adjacent angled struts 745 of a single zig-zag ring 742 (see FIG. 11C). The connections between the angled struts 745 and the diagonal struts 750 are described in more detail below.

The zig-zag rings 742 are formed by a plurality of interconnecting angled struts 745. The angled struts 745 are arranged at uniform, acute angles to one another thereby forming alternating peaks 748 and valleys 751 of the zig-zag rings 742. As an example, a first angled strut 745a connects to a second angled strut 745b forming a proximally-facing peak 748 where their ends meet. Opposite ends of the pair of angled struts 745a, 745b spread away from one another at an angle Θ. The angle Θ between adjacent struts 745 (i.e., the angle of the valley between where the struts 745 connect) can be about 30 to 60 degrees in the deployed configuration, and the angle Θ being smaller (i.e., less than about 30 degrees) in the constrained configuration.

Still with respect to FIG. 11B, each diagonal strut 750 connects on a distal end to the first zig-zag ring 742a and connects on a proximal end to the adjacent zig-zag ring 742b. The zig-zag rings 742a, 742b are formed by pairs of angled struts 745, each pair forming a “V” defining alternating valleys 751 and peaks 748. A peak projecting in a distal direction (in the direction of arrow D) forms a corresponding valley projecting in a proximal direction (in the direction of arrow P). The connection point between a diagonal strut 750 and the angled struts 745 of the zig-zag rings 742 can vary including at a peak 748, at a valley 751, or at a connection point that is mid-strut of angled strut 745. “Mid-strut” as used herein can include a center point of a respective strut or any location along the respective strut between where the strut and a neighboring strut connect to form a peak 748 or a valley 751. An angled strut 745 is connected to at least one diagonal strut 750 of a first section of diagonal struts 750 (i.e., on a distal-facing side of the strut 745) and at least one diagonal strut 750 of a second section of diagonal struts 750 (i.e., on a proximal-facing side of the strut 745. Arrow D of FIG. 11B illustrates a distal direction and Arrow P illustrates a proximal direction. In an exemplary implementation, seen most clearly in detail FIG. 11B, a first angled strut 745a connects to a second angled strut 745b on a first end forming a proximally-facing peak 748p. That proximally-facing peak 748p has a corresponding valley 751d facing in a distal direction, the valley 751d having an angle Θ. The first angled strut 745a also connects to an angled strut 745e at its opposite end forming a distally-facing peak 748d. That distally-facing peak 748d also forms a corresponding valley 751p facing in a proximal direction.

Still with respect to FIG. 11B, the “V” formed by first angled strut 745a and second angled strut 745b of zig zag ring 742a are connected to the “V” formed by third angled strut 745c and fourth angled strut 745d by four diagonal struts 750a, 750b, 750c, 750d. The distal end of first diagonal strut 750a connects to the first angled strut 745a inside the proximally-facing valley 751 formed between the first angled strut 745a and adjacent angled strut 745e. The proximal end of first diagonal strut 750a connects to the third angled strut 745c at a mid-strut point between a distally-facing peak 748 and a distally-facing valley 751. The distal end of second diagonal strut 750b connects to the first angled strut 745a at a mid-strut point between a proximally-facing valley 751 and a proximally-facing peak 748. The proximal end of second diagonal strut 750b connects to the third angled strut 745c at a distally-facing valley 751. The distal end of third diagonal strut 750c connects to the first angled strut 745a at a proximally-facing peak 748. The proximal end of third diagonal strut 750c connects to the fourth angled strut 745d at a mid-strut point between a distally-facing valley 751 and a distally-facing peak 748. The distal end of the fourth diagonal strut 750d connects to the second angled strut 745b at a mid-strut point between a proximally-facing peak 748 and a proximally-facing valley 751. The proximal end of the fourth diagonal strut 750d connects to the fourth angled strut 745d at a distally-facing peak 748. Thus, the diagonal struts 750 connecting one zig zag ring 742a to another zig zag ring 742b create a pattern of connections (e.g., valley to mid-strut, mid-strut to valley, peak to mid-strut, mid-strut to peak, etc.), which will be described in more detail below.

Each angled strut 745 makes connections with diagonal struts 750 on a proximal-facing side of the angled strut 745 as well as on a distal-facing side of the angled strut 745. In the example of FIG. 11B, the same angled strut 745a is connected mid-strut on a proximal-facing side to at least one diagonal strut 750b of one section of diagonal struts and connected mid-strut on a distal-facing side to at least a second diagonal strut 750e of another section of diagonal struts. Similarly, another diagonal strut 750f is connected to angled strut 745a at a distally-facing peak 748 formed where the angled strut 745a connects with a neighboring angled strut 745e above it. The diagonal strut 750f on the distally-facing peak 748 can be positioned opposite the connection between the first diagonal strut 750a within the corresponding proximally-facing valley 751. Another diagonal strut 750g is connected within a distally-facing valley 751 formed where the angled strut 745a connects with its other neighboring angled strut 745b below it. The diagonal strut 750g on the distally-facing valley 751 can be positioned opposite the connection with the third diagonal strut 750c at the corresponding distally-facing peak 748.

The connection points between the peaks 748 and valleys 751 where the diagonal struts 750 connect to the zig-zag rings 742 can be at a central mid-point of the angled struts 745 between an adjacent peak 748 and valley 751. Preferably, the connection point for the strut 750 along the distally-facing side of the angled strut 745 is off-set from the connection point for the strut 750 along the proximally-facing side of the angled strut 745. For example, as shown in FIG. 11B, the mid-strut connection between angled strut 745a and diagonal strut 750e on the distally-facing side of the angled strut 745a is off-set from the mid-strut connection between angled strut 745a and diagonal strut 750b on the proximally-facing side of the angled strut 745a. In contrast, the connection between diagonal strut 750f at the distally-facing peak 748 is directly opposite of the connection between diagonal strut 750a at the proximally-facing valley 751.

As discussed above, first diagonal strut 750a connects the valley 751 of the pair of angled struts 745a, 745e in ring 742a to a connection point of an angled strut 745c of an adjacent ring 742b. The portion of first diagonal strut 750a that is “within” the valley 751 lies substantially parallel to the longitudinal axis A of the flow diverter 700. The first diagonal strut 750a curves away from the longitudinal axis A moving proximally from its distal end to extend substantially diagonal to the axis A (and, in the rolled up actual device configuration, a helical path). The proximal end of first diagonal strut 750a curves again in the opposite direction to be once again parallel to the longitudinal axis A of the flow diverter 700 and positioned within a valley 751 of the adjacent zig-zag ring 742b. In a similar fashion, the first portion of the second diagonal strut 750b that connects between the peak 748 and valley 751 of the pair of angled struts 745 in ring 742a connects to a valley 751 in ring 742b. The third diagonal strut 750c that connects at a peak 748 of the pair of angled struts 745a, 745b in ring 742a has a connection point between a peak 748 and valley 751 of a pair of angled struts 745c, 745d in ring 742b. Each of the diagonal struts 750 incorporates a curve near each end so that at least a portion of each diagonal struts 750 lies parallel to the longitudinal axis A of flow diverter 700 near its connection point with its respective angled strut 745. The diagonal struts 750 continue around the circumference of flow diverter 700 such that angled struts 745 of ring 742a are connected to angled struts 745 of adjacent ring 742b via the diagonal struts 750. The connections formed by the diagonal struts 750a, 750b, 750c, 750d between the adjacent zig-zag rings 742a, 742b create a circumferential pattern of connections. The pattern illustrated in FIGS. 11A-11D includes: 1. Proximally-facing valley of ring 742a connected to mid-strut point of ring 742b; 2. Mid-strut point of ring 742a connected to distally-facing valley of ring 742b; 3. Proximally-facing peak of ring 742a connected to mid-strut point of ring 742b; 4. Mid-strut point of ring 742a connected to distally-facing peak of ring 742b. The pattern of connections repeats around the circumference and along the length of flow diverter 700 in the dense portion 701. The pattern of connections can vary. The pattern illustrated in FIGS. 1A-1B and 2A-2B and 10A-10B includes a single diagonal strut 750 connecting peak-to-peak between angled struts 745 of adjacent rings 742. The single diagonal strut 750 can alternatively connect peak-to-valley between angled struts 745 of adjacent rings 742 or valley-to-valley between angled struts 745 of adjacent rings 742. In another variation, adjacent rings 742 are connected by more than one diagonal struts 750, including a pattern of four diagonal struts 750 connecting each angled struts 745 of adjacent rings 742 as illustrated in FIGS. 11A-11B or a pattern of three diagonal struts 750 connecting each angled struts 745 of adjacent rings 742 or a pattern of two diagonal struts 750 connecting each angled struts 745 of adjacent rings 742. Where the pattern involves one, two, three, or four diagonal struts connecting adjacent rings 742, the connections can be any of a variety of combinations including peak-to-peak, valley-to-valley, peak-to-valley, valley-to-peak, peak-to-midstrut, midstrut-to-peak, valley-to-midstrut, midstrut-to-valley, midstrut-to-midstrut, etc.

Regardless the pattern of the connections between diagonal struts 750 and angled struts 745 of the zig-zag rings 742, each diagonal strut 750 has a substantially identical length from distal end connection to proximal end connection. The substantially identical length of each diagonal strut 750 allows the implant to be constrained in a tubular shape without placing any of the struts 750 under tension. Additionally, the strut 750 curvature at each of the distal and proximal ends of the diagonal struts 750 helps the geometry of the flow diverter 700 crimp to a smaller outer dimension when in a collapsed, delivery configuration (see FIG. 11D). The angled struts 745 of the zig-zag rings 742 compress to a smaller outer diameter without twisting. The ends of the diagonal struts 750 that form connections with the angled struts 745 are substantially parallel to the longitudinal axis A of the flow diverter 700 and the region of the diagonal struts 750 between the ends twist to compress (see FIG. 11D).

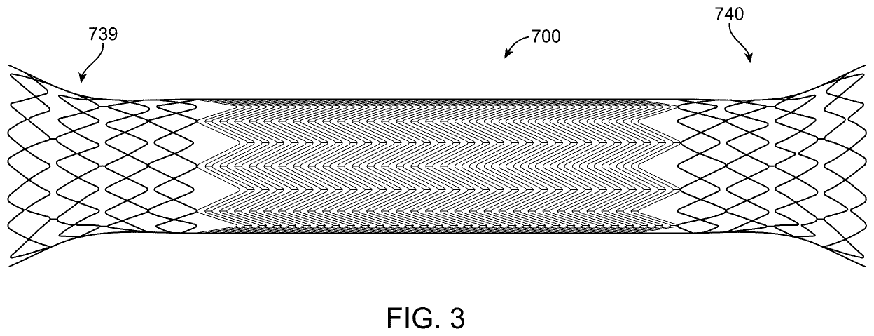

For all implementations of flow diverter 700 described herein, the ends of the flow diverter can be flared during manufacture of the device. For example, as seen in FIG. 3, flow diverter 700 has flared distal end 740 and flared proximal end 739. The flare angle may vary from 15 degrees to 40 degrees, or about 20 to 30 degrees relative to a longitudinal axis of the flow diverter 700 from the proximal end to the distal end. The purpose of the flared shape is to ensure good apposition of the flow diverter ends to the vessel wall even if the device is deployed in a curve. The springy nature of nickel titanium (NiTi) cut tube devices causes the device with flared ends when positioned within a curve to press against the outside of the curve. A device with no flared ends may lift off the vessel wall on the inside of the curve. Either end of the device, depending on the position of the device in the curve, can lift off the wall. If the end is lifted off, it may cause problems with subsequent device advancement or retraction through the flow diverter, as well as increase the risk of device thrombosis.

The flare at the proximal end of the flow diverter can be greater than the flare at the distal end of the flow diverter. For example, the angle of the flare at the proximal end can be about 40 degrees and the angle of the flare at the distal end can be about 20 degrees such that the diameter of the opening into the stent lumen on the proximal end can be about 10 mm and the diameter of the opening into the stent on the distal end can be about 7 mm. The outer diameter within a central region of the stent can be about 4.25 mm, in comparison. The length of the flare on the distal and proximal ends can each be about 3-4 mm long and the non-flared uniform OD region can be about 20-23 mm long.

The length of middle flow diversion zone 701 can be about 10 mm-20 mm. The length of each of the end zones 703, 705 can be about 5 mm-10 mm for a total length of about 18 mm-35 mm, preferably about 20 mm-30 mm. The number of nesting struts around the circumference of the stent can be greater than 12 such as about 16 so as to align with 16-strut geometry of the anchor region. The gap between struts in the middle flow diversion zone can be about 33-34 microns.

The flow diverters described herein can incorporate radiopaque marker receptacles to one or more of the end features and/or features located. For example, the flow diverter may have round or other shaped receptacles 770 cut into the laser pattern of some or all peaks of zig-zag pattern ring 749a, as seen in FIG. 1C. A disk or rivet made of radiopaque material (e.g., gold, platinum, tantalum, or the like, or an alloy of one or more) with a corresponding shape to the receptacle 770 may be pressed, glued, soldered, welded, formed, or otherwise affixed to receptacle 770. Alternately, the flow diverter may have features that allow a radiopaque component such as a section of tube to be secured in a locking fashion to the ends of flow diverter 700. For example, as seen in FIG. 1C and FIG. 1D, flow diverter 700 may have locking features 760 which allow a radiopaque tube marker 775 to be placed on the flow diverter 700 and then formed so it is captured between arrowhead 765 and stopper feature 767. Alternatively, because the flow diverter 700 is manufactured from spring material, arrowhead 765 can be compressed to allow insertion into a pre-formed tube marker 775 and then released to lock marker 775 into place. The shape of arrowhead 765 may vary as long as the design intent is met. The cross section of tube marker 775 may be round, or, in a preferred embodiment is formed to be oval or kidney shaped to reduce the amount of protrusion beyond the ID and OD of flow diverter 700. The tube is constructed from radiopaque material (e.g., gold, platinum, tantalum, or the like, or an alloy of one or more).

The radiopaque markers may also be used to delineate transitions from one density section to another. For example, a first set of one or more receptacles 770a can be positioned at the proximal and distal ends of the flow diverter and a second set of one or more receptacles 770b can be positioned to identify the length Lc such as at either end of high-density zone 701. The receptacles 770a can be positioned at the distal-most and proximal-most ends whereas receptacles 770b can be positioned on the valleys of the high-density zone 701 to identify that densest coverage. Radiopaque material can be pressed into the receptacles 770 to make the ends and zones of the flow diverter 700 visible under fluoroscopy. For example, radiopaque markers can be located in receptacles 770b on either end of the high-density zone 701 so that the user is able to confirm the location of the high-density zone 701 with approximately 30% material coverage is appropriately located across the aneurysm neck, and/or perform procedural steps to ensure that this is true. The arrangement of the receptacles 770 can vary and a few are shown in FIG. 1B as an illustration and is not intended to be limiting as the receptacles 770 can be positioned in any of a variety of locations depending on which portions of the flow diverter are desired to be visualized. At least one of the proximal end zone, the middle zone, and the distal end zone can include at least one radiopaque marker.

The flow diverters described herein may have a specialized antithrombotic surface modifications or coatings, for example, heparin coatings, hydrophilic polymer coatings, such as phosphorylcholine and phenox hydrophilic polymers, albumin, fibrin, and the like.

### Expandable Implant Delivery Systems

Expandable implants, such as stents, coils, and flow diverters are conventionally mounted on an inner delivery core wire and delivered through a microcatheter having an inner diameter of 0.027″ (0.7 mm). In order to be delivered through such a small-sized delivery system while still providing the desired wall coverage (approximately 30%) when expanded in vessel up to 5.0 mm diameter, implants like flow diverters conventionally have braided wire construction.

The delivery of conventional braided implants, such as flow diverters, typically occurs over several procedural steps. First, a microcatheter is inserted into the vasculature and advanced over a guidewire to a position across the target implant site. The microcatheter tip is often placed far distal to the ultimate target implant site because of the imprecise nature of delivering braid-style implants. Once the microcatheter is in position relative to the target implant site, the guidewire is removed. The braided implant is then inserted to the proximal end of the microcatheter using an introducer tube. The implant is pre-mounted on a delivery core wire with features to keep the implant both restrained in the collapsed configuration and secured longitudinally onto the delivery core wire. For example, the core wire can have PTFE sleeves that cover and constrain the braided implant at either end. The core wire often has a distal flexible tip that extends up to 15 mm beyond the distal end of the implant. This means that the distal tip needs to be positioned at least 15 mm beyond the treatment site, and possibly more if the microcatheter is positioned distally, for the implant to be implanted in the correct location, another source of potential complication. The core wire is used to push the implant to the end of the microcatheter. The microcatheter is then retracted to expose the braid, which, by its material properties and construction, begins to spring open. The distal end does not reach its full opening diameter until several millimeters of the braid are exposed due to the nature of the braided construction. The user must often push on the microcatheter while pulling on the core to “push” the braid to its maximum opening in order to get full apposition of the implant against the vessel wall, which is highly desirable to achieve the intended clinical effect. This push and pull technique is yet another potential cause of clinical complication of conventional braided implant as well as adding time to the procedure and imprecision in the implantation location. Braids by their nature shorten considerably upon expansion, making accurate implantation yet more difficult. Often, the implant is delivered distal to the desired site and then partially deployed and “dragged back” into place across the target site. Both the distal positioning of the microcatheter and the “drag back” step are risks for vessel damage and vessel perforation, both leading to severe clinical sequelae.

In many implant delivery systems, the delivery core wire has features that constrain the braid wire ends. The microcatheter following expansion of the implant is fully proximal to the implant and must be re-advanced through the braid to cover the delivery core wire features so that the delivery core wire does not get snagged by the just-deployed implant. Each of these steps potentially disrupt the implant, add to procedural time, and are potential causes of clinical complications due to the extra catheter maneuvering.

The implants described herein can be delivered by delivery systems that are larger in diameter and configured to be used with larger-bore access systems compared to conventional braided-style implants. The delivery systems described herein can be used with any of the above flow diverters described previously including laser cut, braided, or woven flow diverters, or combinations thereof.

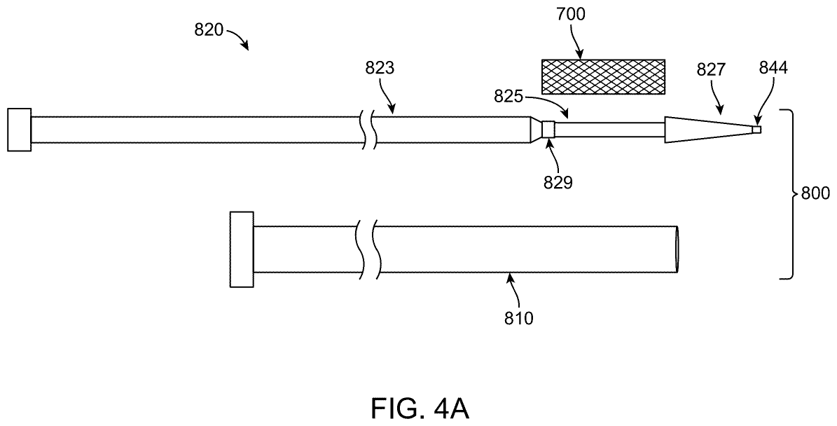

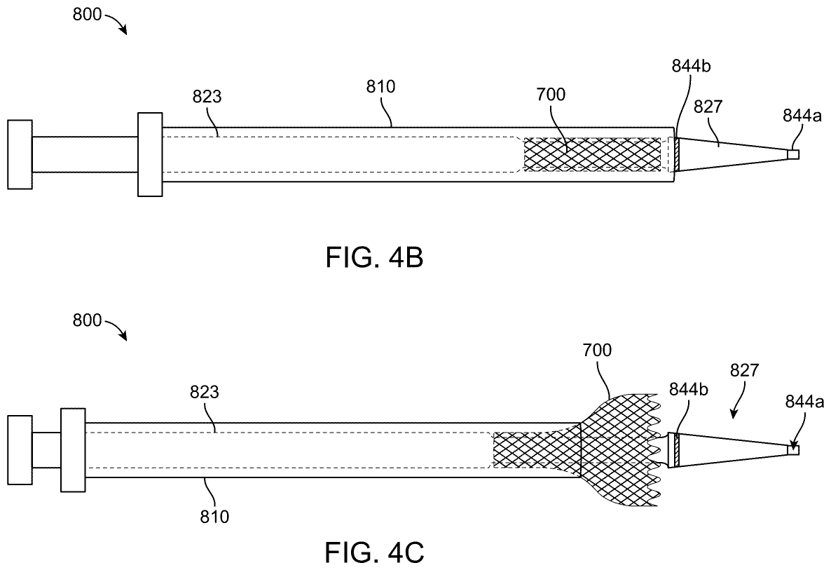

FIGS. 4A-4C illustrate an implant delivery system 800, which can be delivered through an access sheath or access catheter as described elsewhere herein.

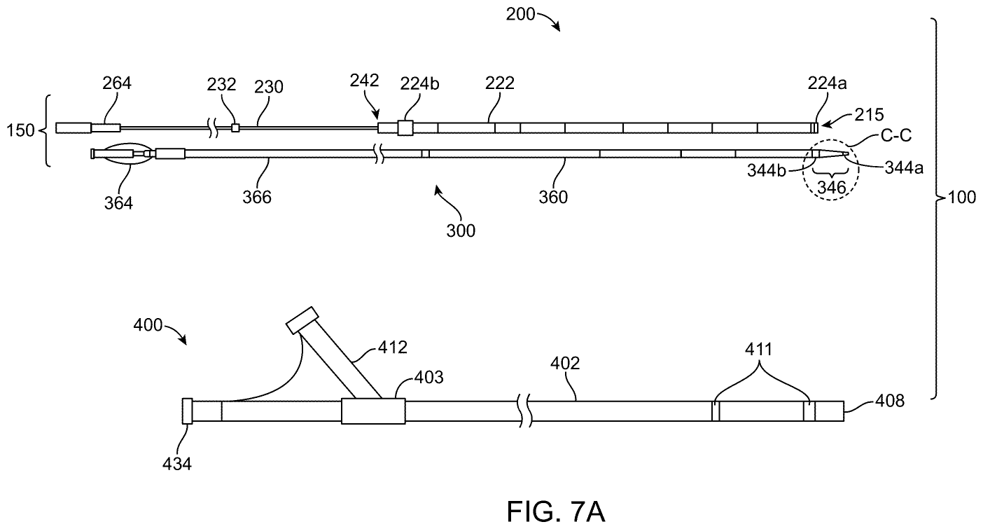

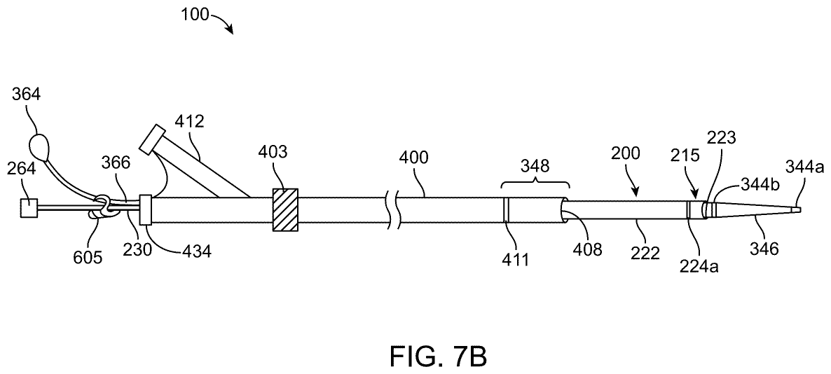

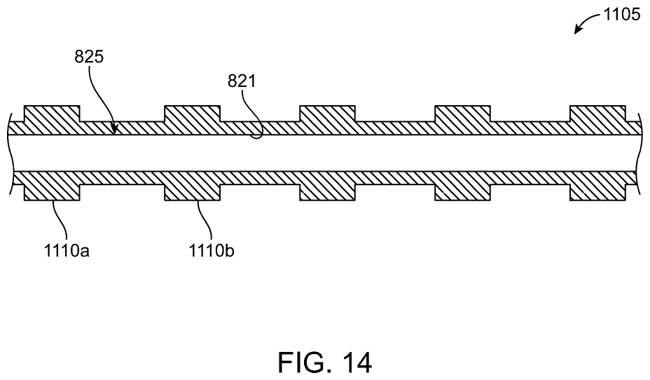

The implant delivery system 800 can include an outer restraining sleeve 810 (or have no separate restraining sleeve and instead another catheter, such as access catheter 200 of FIGS. 7A-7B to be discussed in further detail below, functions as the restraining sleeve for the expandable device, as discussed in more detail below) and an inner core member 820 having an elongate shaft 823 and tapered tip 827. The inner core member 820 can have an inner lumen 821 sized to accommodate a guidewire (shown in FIG. 14). For example, the inner diameter of the core member 820 can be at least about 0.014″ up to about 0.024″, preferably about 0.019″ to about 0.022″ to accommodate a small interventional guidewire that is about 0.014″-0.018″. The lumen can be a single, central lumen that allows the implant 700 and implant delivery system 800 to be delivered over a guidewire, if desired. Alternatively, the delivery system 800 can be delivered without a guidewire, such as using the tip 827 as an atraumatic leading component or delivery tool. And still further, the delivery system 800 can be delivered with a guidewire, but the guidewire remains parked inside the delivery system 800 so that it is available for use, but need not be used or leading the advancement of the delivery system 800. The shaft 823 of the inner core member 820 has a reduced diameter recessed section 825 near a distal end region that is sized to accommodate an implant 700. The implant delivery system 800 can be used to deliver an implant 700, including any of the flow diverters described herein, such as those shown in FIGS. 1A-1D, 2A-2B, 3, 10A-10B, 11A-11D. As shown in FIG. 4B, the implant 700 is positioned in the recess 825 of the inner core member 820 and is retained in this position by the outer restraining sleeve 810. The implant 700 is held by the inner core member 820 within the recessed section 825 and deployed by expansion upon withdrawing the restraining sleeve 810 proximally. The implant delivery system 800 can incorporate one or more locking features to ensure the implant 700 avoids being pulled proximally relative to the inner core member 820 upon withdrawal of the restraining sleeve 810 during deployment. The locking feature can include one or more grip features, or locking features as described herein. For example, the inner core member 820 can include a grip feature 829 located at a proximal end of the recessed section 825 that is configured to prevent the implant 700 from being dragged back over shaft 823 of the inner core member 820 as the restraining sleeve 810 is withdrawn during implant deployment. The grip feature 829 can be a high friction component, such as a length of thin-walled silicone or other elastomeric tube. The locking feature(s) allow for the implant to be re-sheathed during and/or after deployment of the implant.

The materials of the shaft 823 of the inner core member 820 are selected to maintain axial integrity during deployment of the implant 700. For example, the shaft 823 and recessed section 825 can be constructed from one or more polymers of suitable durometer, such as Pebax 55D, Pebax 63D, and/or Pebax 72D. The shaft 823 and/or recessed section 825 can incorporate one or more non-polymeric materials including stainless steel or nitinol, braid-, coil-, cut-hypotube, or otherwise reinforced to provide axial stiffness.

The length of the outer restraining sleeve 810 is shorter than the inner core member 820 by an amount that allows the implant 700 to be fully deployed when the restraining sleeve 810 is pulled back with respect to the inner core member 820 (see FIG. 4C). The restraining sleeve 810 is configured so that it is able to be pulled back easily without dragging the implant 700 with it. For example, the restraining sleeve 810 can be constructed with multiple layers including a low friction inner liner, such as PTFE or FEP. The restraining sleeve 810 can be braid-, coil-, or cut-hypotube-reinforced so as not to stretch during withdrawal. The restraining sleeve 810 can also have an outer hydrophilic coating on the distal portion to improve delivery through a large-bore catheter, which will be described in more detail below. The restraining sleeve 810 can be constructed as described below with respect to catheter 200.

Again, with respect to FIG. 4A, the inner core member 820 can include a distal tip region 827 located distal to the recessed section 825. The distal tip region 827 of the inner core member 820 can be tapered and have a flexibility, a shape, a taper length and a taper angle configured for atraumatic delivery of the delivery system 800 to a vessel in the brain with or without a guidewire.

The distal tip region 827 can have at least one radiopaque marker 844 configured to delineate the tapered section. For example, a first radiopaque marker 844a or first region of a radiopaque marker can identify the distal-most end of the inner core member 820 and a second radiopaque marker 844b or a second region of the radiopaque marker can identify a maximum outer diameter region of the taper. Identification of the maximum outer diameter region of the distal tip region 827 is useful for optimum delivery purposes in that it can be aligned relative to the outer restraining sleeve 810 to minimize a distal-facing lip or edge that would otherwise be created by the sleeve 810. The marker or markers can be rings of radiopaque material, such as gold, platinum/iridium, or other radiopaque material, and embedded, glued or otherwise adhered to the inner core member 820. Alternately, the radiopaque marker can be a polymer impregnated with radiopaque material, such as Pebax impregnated with tungsten, and heat-welded to the inner core member 820. The outer diameter of the inner core member 820 just proximal to the taper is sized to be a smooth fit against the inner diameter of the restraining sleeve 810 so as to present a smooth leading edge to the implant delivery system 800 being advanced in the vasculature with or without a guidewire.

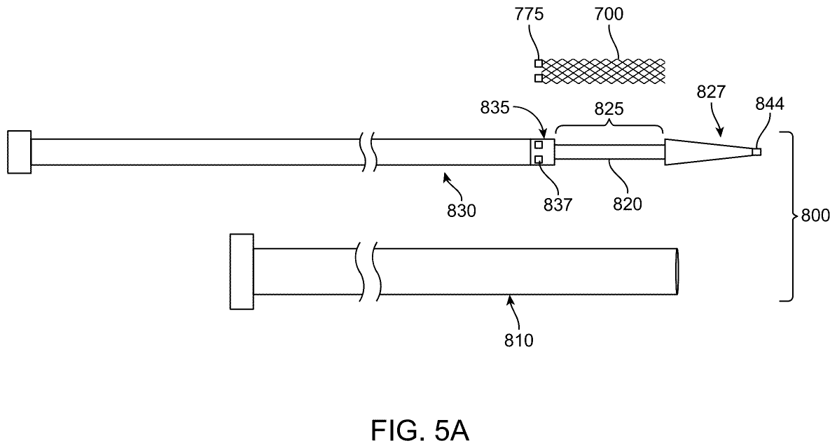

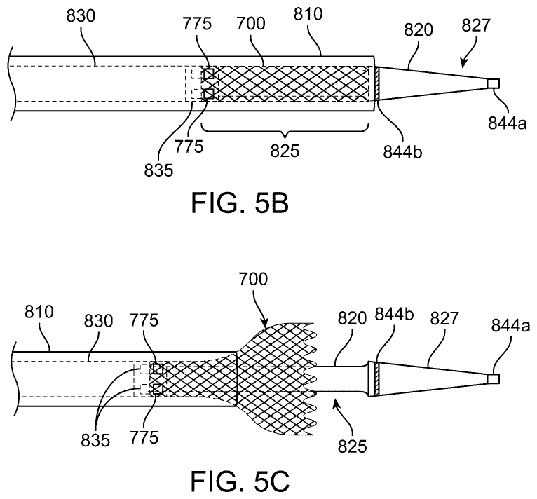

FIGS. 5A-5C illustrate a variation to the implant delivery system 800 of FIGS. 4A-4C that can be used to deliver an implant 700, including any of the flow diverters described herein, such as those shown in FIGS. 1A-1D, 2A-2B, 3, 10A-10B, 11A-11D. As shown in FIG. 5A, the delivery system 800 comprises an outer restraining sleeve 810 and an inner core member 820 as described above with respect to FIGS. 4A-4C. The outer restraining sleeve 810 is optional in that another catheter (access catheter 200) can be utilized to provide the constraining function for the expandable device. Where the restraining sleeve 810 is discussed herein, it should be appreciated that an access catheter 200 can be used to constrain and deploy the device. The delivery system 800 can include a middle member 830 with an engagement component 835 at the distal end. The middle member 830 is positioned axially over inner core member 820, with the distal end terminating proximal to the distal end of inner core member to expose recessed section 825. The inner core member has a distal tip region 827 located distal to the recessed section 825, similar in construction from inner core member of delivery system 800 of FIGS. 4A-4C. The middle and inner member are fixed with respect to each other to create an inner/middle member assembly.

FIGS. 5B and 5C illustrate the deployment of implant 700 from delivery system 800. In FIG. 5B, the implant 700 is positioned in recessed section 825 of inner core member 820. The proximal end of implant 700 with markers 775 overlap the engagement component 835. The outer restraining sleeve 810 is positioned over the inner core member 820, middle member 830 and implant 700, and serves to maintain the position of the implant 700 in its collapsed configuration.

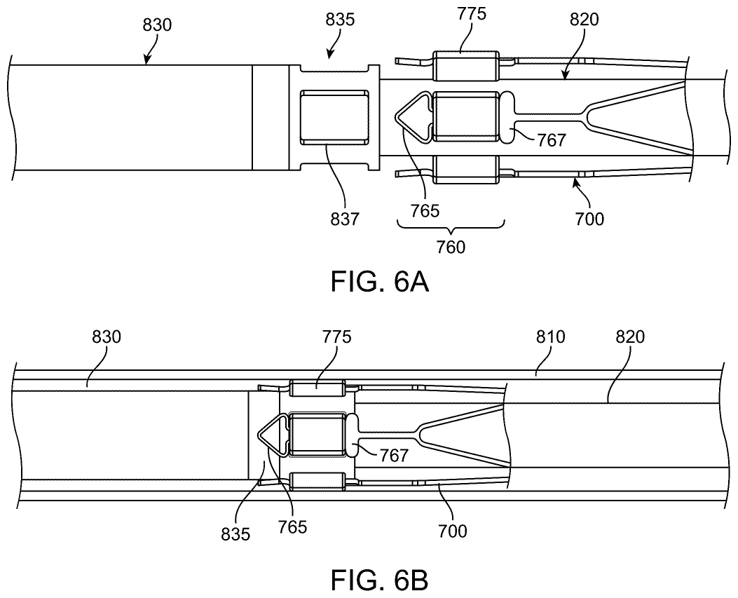

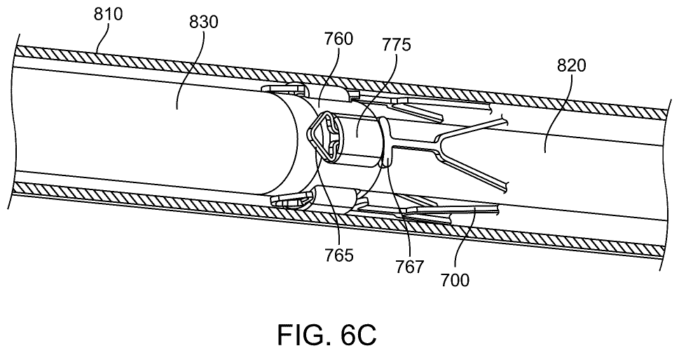

FIGS. 6A-6C are detail views of FIGS. 5A-5C illustrating a locking feature between the implant 700 and the middle member 830. FIG. 6A shows the distal end of middle member 830 in proximity to the proximal end of implant 700. As illustrated, the engagement component 835 has surface features 837, which can be cutouts or recesses. These surface features 837 correspond in size and shape to the markers 775 positioned on the proximal end of implant 700. In an example, there are four surface features 837 on the engagement component 835, corresponding to four markers 775 on the implant 700. FIG. 6B shows the engagement component 835 overlapping with proximal end of implant 700, with outer restraining sleeve 810 holding the markers 775 into surface features 837, thereby locking the implant 700 to the middle member 830. This assembly is seen in isometric view in FIG. 6C.

This locking feature enables the implant 700 to maintain its position on inner/middle member assembly while the restraining sleeve 810 is pulled back or pushed forward to either deploy or re-sheath the implant during placement of the implant in the desired anatomic position. This same locking feature also aids in assembly of the implant to the delivery system by allowing the implant to be pulled into the delivery system during manufacture of the implant and delivery system assembly. The locking feature of FIGS. 5A-5C and 6A-6C can be incorporated alone or together with the grip feature 829 shown in FIGS. 4A-4C.

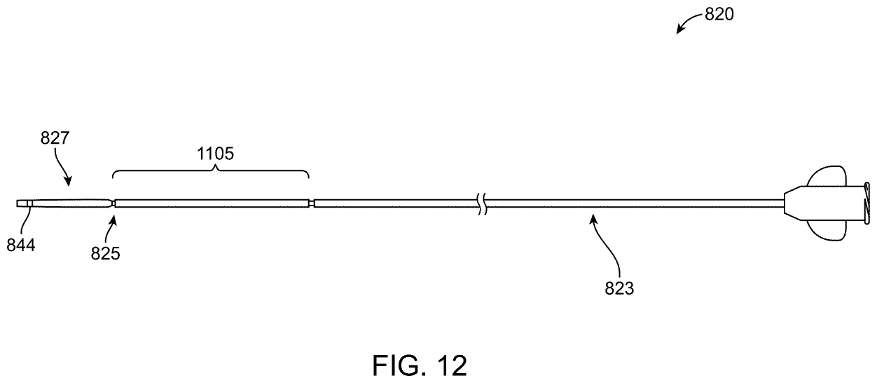

The locking features described above are designed to engage a proximal end region of the implant 700. The delivery systems 800 described herein can incorporate a locking feature that is designed to engage with more than just the proximal end region of the implant 700. For example, the locking features can engage with a distal end region, a middle, and/or the proximal end region of the implant 700. The locking feature can grip the implant 700 along the entire length of the implant 700. A variation of inner core member 820 of the delivery system 800 of FIGS. 4A-4C, 5A-5C is shown in FIG. 12. In addition to elongated shaft 823 and tapered tip 827, the inner core member 820 includes locking feature that is formed by a soft grip portion 1105 located on elongate member 823 where the implant 700 is positioned during delivery through the delivery sheath 810, such as over the recessed section and under the implant 700. The grip portion 1105 serves to engage or grip the implant 700 along the entire length of the implant so that during retraction of the sheath 810 to deploy the implant 700, or re-advancement of the sheath 810 to re-compress and reposition the implant 700, the implant 700 is neither elongated nor compressed in length due to movement of the sheath 810. This is especially valuable with implant designs that can stretch out when in tension, or shorten in compression, such as the implant designs in FIGS. 1A-1B, 2A-2B, 3, 10A-10B and 11A-11D.

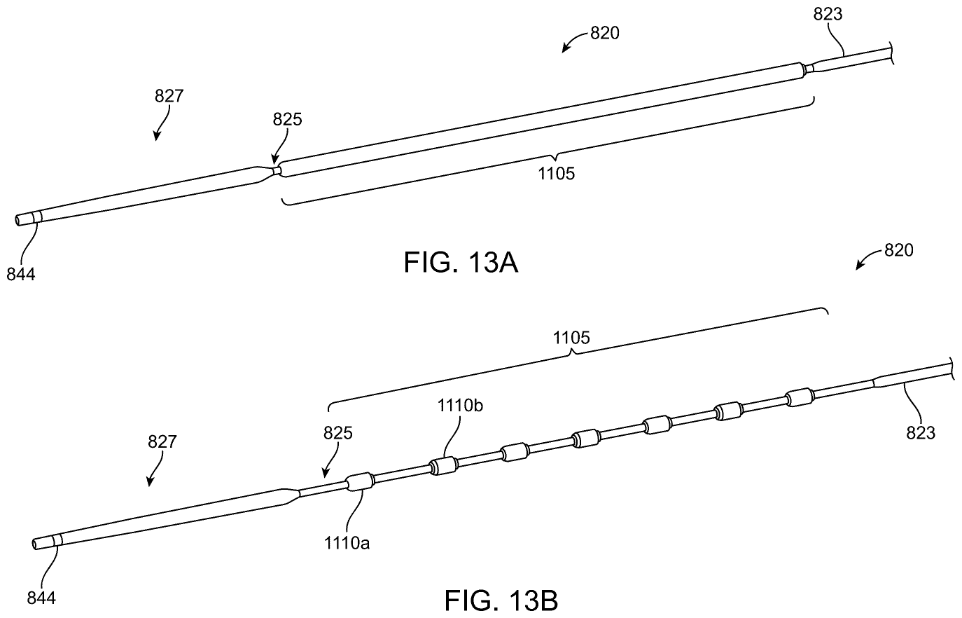

FIG. 13A is a detail view of the grip portion 1105, which is shown as a single component of uniform diameter over its length. The grip portion 1105 may also be formed of several discreet grip segments (i.e., 1110a, 1110b, and so on) as shown in FIG. 13B. FIG. 13B illustrates 7 grip segments along a length of the recessed section 825 that are equally spaced. The grip portion 1105 can include 1, 2, 3, 4, 5, 6, 7, 8, 9, 10, or more grip segments and the grip segments can be the same length or different lengths, the same outer diameter or different outer diameter, evenly spaced or unevenly spaced. These grip segments 1110a, 1110b serve to grip the implant 700 just in specific points along the length of the implant 700. The locations of the grip segments 1110a, 1110b can coincide with features between the sections that can stretch or compress. For example, in the case of a flow diverter of FIGS. 1A-1B, 2A-2B, 3, 10A-10B or 11A-11D, the inner core member 820 can include a grip portion 1105 made up of distinct grip segments 1110 that are arranged along the recessed section 825 to coincide axially with each circumferential zig-zag ring 742 and each end section 703 and 705. This segmented variation of a grip portion 1105 reduces the compression force of the outer sheath 810 against the implant 700 and inner core member 820, and therefore reduces the force needed to retract the sheath 810 to deploy the device 700.

The delivery systems described herein incorporating a higher friction grip portion, particularly discreet segments that provide higher friction, which are used for expandable implants 700 including closed cell design implants 700 and are particularly useful for open cell design implants 700. For example, the discreet segments of the grip portion can be positioned along the recessed section 825 at locations selected to coincide with target areas of an open cell implant 700, such as at the stent struts, to reinforce those target areas from stretching and/or compressing during pushing and pulling movements. Closed cell design stents and flow diverters are characterized by small free cell areas between the stent struts due to, for example, each peak of the rings being connected to a diagonal strut. This is compared to open cell designs, which have larger free cell areas due to, for example, only a select few of the peaks in a ring being connected to a diagonal strut. Closed cell design typically have greater coverage and radial strength compared to open cell designs and tend to be stiffer to delivery and stiffer once deployed. Open cell design stents and flow diverters are more prone to stretching out when pulled and/or compressing when pushed, whereas closed cell design stents and flow diverters tend to maintain their length with no or minimal stretching or compression during pushing and pulling movements of delivery system components.

The grip segments 1110a, 1110b, and so on, may be separate discreet components arranged over and around different regions of the recessed section 825. Alternatively, the grip segments 1110a, 1110b, and so on, may be formed as one grip component with alternating larger diameter sections. FIG. 14 is a cross-sectional view of a grip portion of an inner core member. The grip portion 1105 is formed of a soft material covering the outer surface of the recessed section 825 of the inner core member 820 with larger outer diameter grip segments 1110 alternating with smaller outer diameter regions. The larger outer diameter grip segments 1110 are sized to engage with an inner surface of the implant 700 and the smaller outer diameter regions between them are sized to not substantially engage with the inner surface. The material for the grip portion 1105 (whether unitary and designed to engage the entire inner surface of the implant 700 or segmented to engage less than an entire inner surface) is a soft material such as low durometer silicone rubber, thermoplastic urethane, or another thermoplastic elastomer. The grip portion 1105f can be molded or extruded separately and adhered to elongated member 823, insert-molded in place, or molded and reflowed in placed (if thermoplastic). Insert molding or reflowing has an advantage in that the soft material can enter the reinforced section (coil or braid gaps or laser cut tube slits) to improve the strength of the adherence. If adhered with a separate adhesive, the adhesive is flexible to maintain the flexibility of the inner core member 820. The grip portion 1105 is made from non-sticky material, to allow for unimpeded deployment of the implant 700, or the grip portion 1105 may be coated, for example with a low friction coating such as paralyne or other hydrophilic coating.

The dimensions of the implant 700 and the implant delivery system 800 are sized to be deliverable through larger-bore access systems. As discussed above, the implant 700 can be a flow diverter with a cut-tube design having zig-zag rings 742 that are connected via diagonal struts 750 according to a circumferential pattern of connections (e.g., peak-to-valley or another pattern as shown in FIGS. 10A-10B and 11A-11D). Upon expansion, the flow diverter has a dense material coverage (e.g., 30% coverage or 70% porosity in the middle zone) due to the tightly nested arrangement of the cells. The flow diverter can take advantage of the constraint of a larger-bore access system to achieve this dense material coverage. For example, for a catheter system 100 having an access catheter 200 with inner diameter (ID) of 0.088″, the outer restraining sleeve 810 can have an outer diameter (OD) of about 0.082″ leaving an annular clearance of 0.003″ (ID/OD difference of 0.006″) for optimal advancement of the implant delivery system 800 through the access catheter 200. In this example, the ID of the outer restraining sleeve 810 is about 0.070″. The collapsed implant 700 can have an OD of about 0.064″ to slide easily through this outer restraining sleeve 810. The inner core member 820 can have an OD of about 0.064″, with the smaller ID recessed section 825 depending on the wall thickness of the flow diverter 700. If the wall thickness of the cut-tube flow diverter 700 is about 0.005″, the recessed section 825 has an OD of about 0.054″.

Larger access systems allow for alternate delivery methodologies. For example, rather than first placing a microcatheter across the target treatment site, removing the guidewire, and then pushing the implant into place as with conventional delivery systems, the implant 700 described herein can be pre-mounted onto the delivery system 800 with the restraining sleeve 810, and delivered to the site through a larger delivery system (e.g., 0.087″-0.126″ ID). The guidewire (if present), implant 700, and inner core member 820 can all be pre-mounted in one system rather than exchanging the guidewire for the implant and inner core member as in conventional systems.

In some implementations, the access catheter 200 acts as the restraining sleeve for the implant delivery system 800 in place of a separate restraining sleeve 810. Thus, the implant delivery system 800 illustrated in FIGS. 4A-4C, 5A-5C, 6A-6C need not include a separate restraining sleeve 810 and can instead utilize the access catheter 200 as its restraining sleeve 810. The implant 700 can be mounted on the inner core member 820 (i.e., around the recessed area 825) and introduced into the catheter system 100 via a separate introducer component and pushed via advancement of the inner core member 820 to the target treatment site in the same manner as current implants may be introduced into microcatheters previously positioned across the target site. In this example, the access catheter 200 can be previously positioned across the target site. Once the implant 700 is positioned at its target site, the inner core member 820 can be held in place while the access catheter 200 is pulled back to deploy implant 700. In this example, there is one “layer” of catheters that is eliminated (i.e., the restraining sleeve 810). This allows for a larger inner diameter for a same size implant. The implant having an outer diameter of 0.064″ can be delivered using an access catheter having an inner diameter of 0.070″ and an outer diameter of 0.082″ (vs. ID 0.088″ and OD 0.100″ of the previous example). The recessed area 825 of the inner core member 820 (see, e.g., FIGS. 4A and 5A) creates a space for the implant 700 during delivery. The inner core member 820 can include a grip section 1105 to hold the implant in place along some or all of the length of the implant 700.

As mentioned above, the inner core member 820 of any of the delivery systems 800 described above can include a distal tip region 827 that is coupled to an elongate shaft 823 of the inner core member 820. The distal tip region 827 projects distal to the region of the inner core member 820 supporting the implant 700 and the elongate shaft 823 extends proximal to the region of the inner core member 820 supporting the implant 700.

The elongate shaft 823 of the inner core member 820 as well as the area of the inner core member 820 supporting the implant 700 is formed of one or more materials that are selected to maintain axial integrity during deployment of the implant 700. The proximal elongate shaft 823 provides a relatively stiff proximal end suitable for manipulating (e.g., advancing and withdrawing through an access system) the delivery system 800 relative to the anatomy and/or the catheter through which it is advanced. The elongate shaft 823 can be formed of one or more less flexible materials than the distal tip region 827 and the flexibility of the elongate shaft 823 can change over its length so that it becomes stiffer towards the proximal end. The proximal elongate shaft 823 can be fully polymeric. The elongate shaft 823 and recessed section 825 can be constructed from Pebax, such as Pebax 72D. The elongate shaft 823 and recessed section 825 can be braid-, coil-, cut-hypotube, or otherwise reinforced to provide axial stiffness. The elongate shaft 823 and recessed section 825 can be metal reinforced up to where it couples with the distal tip region 827. The metal reinforcement can transition as it approaches the coupling with the distal tip region 827 so that the inner core member 820 becomes more flexible at the material transition to the distal tip region 827. As an example, the metal reinforcement can become more flexible by increasing the spacing between coils or other technique to increase flexibility of a reinforced segment of catheter.

At least a portion of the distal tip region 827 of the inner core member 820 has a flexibility configured for atraumatic delivery of the delivery system 800 to a vessel in the brain with or without a guidewire. The distal tip region 827 transitions moving proximally to become less flexible. The distal-most portion of the distal tip region 827 is very flexible and the proximal-most portion of the distal tip region 827 approaches the flexibility of the location where the implant 700 is supported (e.g., recessed section 825).

The distal tip region 827 can be tapered along at least a portion of its length. The maximum outer diameter region can taper distally towards a smaller outer diameter near the distal-most end of the inner core member 820. The maximum outer diameter provides a snug point or close fit between the distal tip region 827 and the outer component (e.g., restraining sheath 810 or access catheter 200, if no separate restraining sheath 810 is used). The maximum outer diameter of the distal tip region 827 minimizes a distal lip or edge at the distal end of the system 800, but still allows for movement between the inner core member 820 and the outer component upon application of a relatively small load for usability purposes. A difference between the inner diameter of the outer component and the maximum outer diameter of the distal tip region 827 can be no more than about 0.015″ (0.381 mm), or can be no more than about 0.010″ (0.254 mm), for example, from about 0.003″ (0.0762 mm) up to about 0.012″ (0.3048 mm), preferably about 0.005″ (0.127 mm) to about 0.010″ (0.254 mm), and more preferably about 0.007″ (0.1778 mm) to about 0.009″ (0.2286 mm).

The proximal elongate shaft 823 can be substantially uniform in outer diameter along at least a portion of its length whereas the distal tip region 827 can taper down to a smaller outer diameter at its distal-most end. The proximal elongate shaft 823 of the inner core member 820 can also change in outer diameter along its length. The proximal elongate shaft 823 of the inner core member 820 can be up to about 150 cm in total length. The recessed section 825 of the inner core member 820 can be at least as long as the implant 700 being delivered, for example about 10 mm-50 mm, preferably slightly longer than the unexpanded length L1 of the implant 700 being delivered. The distal tip region 827 can be about 1 cm to about 20 cm in length including the tapering portion. The distal tip region 827 can be about 5 cm to about 10 cm, including 5, 6, 7, 8, 9, 10 and any length in between. In some implementations, the distal tip region 827 is longer than 10 cm up to about 15 cm, up to about 20 cm, up to about 25 cm and any length in between. The length of the tapering outer diameter of the distal tip region 827 can be about 0.5 cm to about 5 cm, about 1 cm to about 4 cm, or about 1.5 cm to about 3 cm, or between 2.0 cm and about 2.5 cm. The entire working length of the inner core member 820 can vary, but generally is long enough to extend through the catheter it is advanced through (e.g., catheter 200) plus at least a distance beyond the distal end of the catheter to deploy the implant at the treatment site while at least a length of the proximal portion remains outside the proximal end of the guide sheath and outside the body of the patient. In some implementations, the overall length excluding any proximal feature such as a hub or luer can be about 145 to about 170 cm.

The taper of the distal tip region 827 can be a constant taper from the maximum outer diameter down to the second smaller outer diameter. In some implementations, the constant taper of the distal tip region 827 can be from about 0.048″ outer diameter down to about 0.031″ (0.787 mm) outer diameter over a length of about 1 cm. In some implementations, the constant taper of the distal tip region 827 can be from 0.062″ (1.575 mm) outer diameter to about 0.031″ (0.787 mm) outer diameter over a length of about 2 cm. In still further implementations, the constant taper of the distal tip region 827 can be from 0.080″ (2.032 mm) outer diameter to about 0.031″ (0.787 mm) outer diameter over a length of about 2.5 cm. The angle of the taper of the distal tip region 827 can vary depending on the maximum outer diameter. For example, the angle of the taper can be between 0.9 to 1.6 degrees relative to horizontal. The angle of the taper can be between 2-3 degrees from a center line of the elongate body 360. The length of the taper of the of the distal tip region 827 can be between about 0.5 cm to about 5.0 cm.

The distal-most end of the outer component, whether the restraining sheath 810 or the access catheter 200 used to deploy the implant 700, can be blunt and have substantially no change in the dimension of the outer diameter whereas the distal tip region 827 can incorporate a distal taper providing an overall elongated tapered geometry of the delivery system. The maximum outer diameter of the distal tip region 827 approaches the inner diameter of the outer component such that the step-up from the distal tip region 827 to the outer component is minimized. Minimizing this step-up prevents issues with the lip formed by the distal end of the outer component catching on the tortuous neurovasculature, such as around the carotid siphon near the ophthalmic artery branch, when the distal tip region 827 in combination with the distal end region of the outer component bends and curves along within the vascular anatomy.

The taper of the distal tip region 827 tapers distally from the maximum first outer diameter to the smaller second outer diameter. The second outer diameter can be about 20%, 25%, 30%, 35%, 40%, 45%, 50%, 55%, 60%, or about 65% of the maximum outer diameter. The maximum outer diameter can be at least about 1.5 times, 2 times, 2.5 times, or about 3 times larger than the smaller distal outer diameter.

The flexibility of the taper of the distal tip region 827 is flexible enough to navigate tortuous anatomy, for example, vessels leading to the level of M1 or M2 arteries, without kinking and without damaging the vessel. The material properties of the distal tip region 827 in combination with wall thickness, angle of taper, length of the taper can all contribute to the overall flexibility of the distal tip region 827 and its transition towards the stiffer, more proximal sections of the inner core member 820 that are supporting the implant 700.

The taper of the distal tip region 827 can be formed of a single polymeric material that is molded into the tapered shape. For example, the taper of the distal tip region 827 can be formed of a first material having a Shore material hardness of no more than 35D or about 62A, such as 35D Pebax. The first material region can have a length that is at least about 0.5 cm to about 5 cm along the distal tip region 827 from the distal-most terminus of the inner core member 820 proximally. The distal tip region 827 can transition from the first material to at least a second material that is less flexible than the first material. The second material or combination of materials can have a Shore material hardness of no more than 55D, such as a combination of 35D Pebax and/or 55D Pebax. The second material region can have a length along the distal tip region 827 proximal of the first material region. The distal tip region 827 can transition from the second material to at least a third material or combination of materials that is less flexible than the second material. The third material or combination of materials can have a Shore material hardness of no more than 65D, such as 55D Pebax. The distal tip region 827 can transition from the third material to at least a fourth material or combination of materials that is less flexible than the third material. The fourth material or combination of materials can have a Shore material hardness of no more than 72D. The fourth material region can be substantially matched in Shore material hardness to the material properties of the recessed section alone or together with the implant 700 mounted around the recessed region to mitigate kinking between the extremely flexible distal tip formed of very soft materials (e.g., 35D Pebax) to the less flexible region where the implant 700 is mounted.

The transition from the extremely flexible distal-most tip of the delivery system 800 to the much stiffer region where the implant is housed can vary in materials, lengths of materials, location and number of material transitions to ensure the change in flexibility avoids kinking. The materials used to form the regions of the distal tip region 827 described above are just examples and can vary, including one or more polyether block amides (PEBAX), such as PEBAX 77A, 25D, 33D, 35D, 42D, 46D, 54D, 55D, 69D, 72D, 90D or a blend of PEBAX (such as a mix of 25D and 35D, 25D and 55D, 25D and 72D, 35D and 55D, 35D and 72D, 55D and 72D, where the blend ratios may range from 0.1% up to 50% for each PEBAX durometer), with a lubricious additive compound, such as Mobilize (Compounding Solutions, Lewiston, Maine). In some implementations, the material used to form a region of the distal tip region 827 can be Tecothane 62A. Incorporation of a lubricious additive directly into the polymer means incorporation of a separate lubricious liner, such as a Teflon liner, is unnecessary. However, the inner core member 820 may also incorporate a liner along all or at least some of its length. The number of transitions can vary including 1, 2, 3, 4, or more material transitions from the distal-most end of the implant delivery system to the location of the implant. For example, the distal tip region 827 can include at least 3 material transitions and up to about 5 material transitions along its length from a distal-most end of the elongate shaft to the recessed section. As discussed elsewhere herein, the material transitions can incorporate a change in outer diameter such that the distal-most outer diameter is smaller than the outer diameter near the implant. Alternatively, the material transitions along the distal tip region can incorporate a rounded or “bullet-nosed” tip or other shape. And still further, the material transitions along the distal tip region can incorporate no engineered change in outer diameter or shape such that the distal tip region extending distal to the location of the implant of the implant delivery system 800 is substantially uniform along its length. The flexibility over the distal tip region whether tapered, rounded, bullet-nosed, or uniformly cylindrical can transition in flexibility from being extremely soft and atraumatic and have a very low bending stiffness at the distal-most end to be substantially similar to the flexibility or bending stiffness of the region of the implant delivery system where the implant is located. The transition in flexibility of bending stiffness along the distal end region of the implant delivery system distal to the implant to the location of the implant can be due to changes in material durometer along the distal end region where changes from one segment to the next segment are within, for example, 20 Shore, 15 Shore, or 10 Shore, or less providing a transition along the length that is so slight and so smooth that the distal end region of the system is able to navigate turns (including corkscrew turns or turns of 180 degrees) within the bony intracranial anatomy without kinking and to follow the flow of blood through the vessels towards the target site without potential for dissection and/or puncture.

The reinforcement layer of the inner core member 820 can terminate prior to the taper of the distal tip region 827 such that at least the taper has a flexibility provided by the fully polymeric materials that are soft and flexible and capable of articulating and bending more easily that the region of the core member 820 where the flow diverter is positioned. The bending stiffness of the distal tip region 827 transitions towards where the flow diverter is positioned to thereby approach the bending stiffness of this region so that no kink points are created along the length of the distal tip region 827 where it transitions toward the stiffer parts of the delivery system 800.

### Access Systems

The implant and implant delivery systems described above can be delivered through an access system and/or access catheter with an appropriately large-bore inner diameter and the ability to reach the target treatment site. Current access devices, i.e., guide catheters and/or guide sheaths, are used to access neurovascular anatomy with limitations.

Guide catheters or guide sheaths are used to guide interventional devices to the target anatomy from an arterial or venous access site, typically the femoral artery or vein, but also including radial, ulnar, or brachial arteries and veins. The access site can additionally incorporate a direct puncture of the carotid artery or jugular vein. The length of the guide is determined by the distance between the access site and the desired location of the guide distal tip. Interventional devices, such as guidewires, microcatheters, and intermediate catheters used for sub-selective guides, are inserted through the guide and advanced to the target site. Often, devices are used in a co-axial fashion, namely, a guidewire inside a microcatheter inside an intermediate catheter, and advanced as an assembly to the target site in a step-wise fashion with the inner, most atraumatic elements, advancing distally first and providing support for advancement of the outer elements. The length of each element of the coaxial assemblage takes into account the length of the guide, the length of proximal connectors on the catheters, and the length needed to extend from the distal end.

Typical tri-axial systems, such as for delivery of flow diverters, stents, stent retrievers and other interventional devices, require overlapped series of catheters, each with their own rotating hemostatic valves (RHV) on the proximal end. For example, a guidewire can be inserted through a Penumbra VELOCITY microcatheter having a first proximal RHV, which can be inserted through a Penumbra ACE68 having a second proximal RHV, which can be inserted through a Penumbra NEURONMAX 088 access catheter having a third proximal RHV positioned in the high carotid via a femoral introducer. Maintaining the coaxial relationships between these catheters can be technically challenging. The three RHVs must be constantly adjusted with two hands or, more commonly, four hands (i.e., two operators). Further, the working area of typical tri-axial systems for intracranial and cerebral device delivery can require working area of 3-5 feet at the base of the operating table. Time is required to access the treatment site using tri-axial systems.

There is also difficulty in getting larger-bore access catheters and sheaths in a rapid and atraumatic fashion to intracranial and cerebral vessels. Both the lengths and diameters of current systems put limitations on the delivery system of endovascular scaffolding devices, such as stents, or flow diverters, which in turn limits the safety, speed, and precision of delivering such devices. There is a need for a system of devices and methods that allow for rapid access of distal intracranial and cerebral vessels with larger lumen sizes and/or shorter lengths.

The access systems and methods described herein enable safe and rapid positioning of large interventional devices, such as the implant delivery systems of FIGS. 4A-4C, 5A-5C, 6A-6C, 12, 13A-13B, 14, 15 to a treatment site in an intracranial or cerebral artery or vein or venous sinus. Further, the extreme flexibility and deliverability of the distal access catheter systems described herein allow the catheters to take the shape of the tortuous anatomy rather than exert straightening forces creating new anatomy. The distal access catheter systems described herein can pass through tortuous loops while maintaining the natural curves of the anatomy therein decreasing the risk of vessel straightening. The distal access catheter systems described herein can thereby create a safe conduit through the neurovasculature maintaining the natural tortuosity of the anatomy for other catheters to traverse (e.g., interventional device delivery catheters). The catheters traversing the conduit need not have the same degree of flexibility and deliverability such that if they were delivered directly to the same anatomy rather than through the conduit, would lead to straightening, kinking, or folding of the anterior circulation.

Provided herein are access systems including a catheter advancement element having a tapered distal end region with a flexibility, shape, and taper length configured to be atraumatically delivered to a vessel in the brain. This is not achieved with conventional catheter systems as they may have improper flexibility, are formed of improper materials, or have improper shape and/or taper length resulting in conventional catheter systems getting misdirected or hung up or, if more force is applied, perforating the vessel. Unlike these conventional catheter systems, the catheter systems described herein includes a catheter advancement element capable of safely navigating neurovascular anatomy and find the lumen so that a corresponding large bore catheter (i.e., implant delivery system) can be delivered to distal sites. The catheter systems described herein help locate occlusions in the vessels in the novel manner of the methods provided herein. These and other features will be described in detail herein.

FIGS. 7A-7B illustrate an implementation of a catheter system 100 including one or more devices for accessing and/or treating an intracranial or cerebral vessel, such as an aneurysm by deploying a flow diverter or an area of blood vessel narrowing by deploying a stent. FIG. 7A is an exploded view of an implementation of a catheter system 100 and FIG. 7B is an assembled view of the catheter system 100 of FIG. 7A. The catheter system 100 can include a catheter 200 alone or together with catheter advancement element 300. The catheter system 100 can include catheter advancement element 300 alone. The catheter system 100 can additionally incorporate access guide sheath 400. The catheter system 100 can additionally incorporate an implant delivery system 800 as described elsewhere herein, alone or together with an implant 700. Any combination of catheters is considered herein when referring to the catheter system 100.

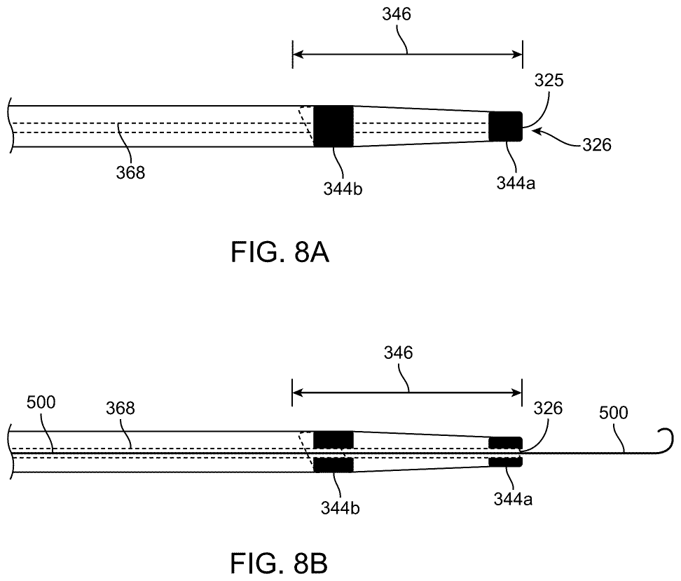

FIG. 8A is a detailed view of the catheter advancement element 300 of FIG. 7A taken along circle C-C. FIG. 8B is a detailed view of a catheter advancement element having a guidewire 500 in the lumen 368 so that the distal end of the guidewire 500 extends distal to the distal opening 326 of the lumen 368. The catheter system 100 is capable of providing quick and simple access to distal target anatomy, particularly the tortuous anatomy of the intracranial and cerebral vasculature. The catheter system 100 can be a single operator system such that each of the components and systems can be delivered and used together by one operator through a single point of manipulation requiring minimal hand movements. As will be described in more detail below, all wire and catheter manipulations can occur at or in close proximity to a single rotating hemostatic valve (RHV) or more than a single RHV co-located in the same device.

The catheter system 100 can include one or more catheters (200, 300, 400, 800) or catheter systems 150 having a catheter 200 and a catheter advancement element 300. The catheter system 150 is configured to be advanced through an access guide sheath 400. The catheter 200 is configured to be received through the guide sheath 400 and is designed to have exceptional deliverability. The catheter 200 can, but need not, be a distal access catheter having a distal tubular component 222 coupled to a smaller outer diameter proximal control element 230. The distal tubular component 222 being co-axial with a lumen of the guide sheath 400 provides a step-up in inner diameter within the conduit. The catheter 200 need not include the proximal control element 230 and instead can be a conventional, full-length catheter having a uniform outer diameter between the proximal and distal ends.

The catheter 200 can be delivered using a catheter advancement element 300 inserted through a lumen 223 of the catheter 200. The flexibility and deliverability of the distal access catheter 200 allow the catheter 200 to take the shape of the tortuous anatomy and avoids exerting straightening forces creating new anatomy. The distal access catheter 200 is capable of this even in the presence of the catheter advancement element 300 extending through its lumen. Thus, the flexibility and deliverability of the catheter advancement element 300 is on par or better than the flexibility and deliverability of the distal luminal portion 222 of the distal access catheter 200 in that both are configured to reach the middle cerebral artery (MCA) circulation without straightening out the curves of the anatomy along the way.

Still with respect to FIGS. 7A-7B, the catheter system 100 can include an access guide sheath 400 having a body 402 through which a working lumen extends from a proximal hemostasis valve 434 coupled to a proximal end region 403 of the body 402 to a distal opening 408 of a distal end region. The working lumen is configured to receive the catheter 200 therethrough such that a distal end of the catheter 200 can extend beyond a distal end of the sheath 400 through the distal opening 408. The guide sheath 400 can be used to deliver the catheters described herein as well as any of a variety of working devices known in the art. For example, the working devices can be configured to provide thrombotic treatments and can include large-bore catheters for delivery of implants, including stents or flow diverters and the like.

The sheath body 402 can extend from a proximal furcation or rotating hemostatic valve (RHV) 434 at a proximal end region 403 to a distal end opening 408 of the body 402. The proximal RHV 434 may include one or more lumens 412 molded into a connector body to connect to the working lumen of the body 402 of the guide sheath 400. The working lumen can receive the catheter 200 and/or any of a variety of working devices for delivery to a target anatomy. The RHV 434 can be constructed of thick-walled polymer tubing or reinforced polymer tubing. The RHV 434 allows for the introduction of devices through the guide sheath 400 into the vasculature, while preventing or minimizing blood loss and preventing air introduction into the guide sheath 400. The RHV 434 can be integral to the guide sheath 400 or the guide sheath 400 can terminate on a proximal end in a female Luer adaptor to which a separate hemostasis valve component, such as a passive seal valve, a Tuohy-Borst valve or RHV may be attached. The RHV 434 can have an adjustable opening that is open large enough to allow removal of devices that have adherent clot on the distal end opening 408 without causing the clot to dislodge at the RHV 434 during removal. Alternately, the RHV 434 can be removable, such as when a device is being removed from the sheath 400, to prevent clot dislodgement at the RHV 434. The RHV 434 can be a dual RHV or a multi-head RHV.

Contrast agent can be injected through the guide sheath 400 into the vessel to visualize the occlusion site by angiogram. For example, the guide sheath 400 can be positioned so that at least a portion is positioned within the carotid artery. The contrast agent may be injected through the sheath 400 once positioned in this location. Contrast agent can also be injected through one or more catheters inserted through the guide sheath 400. A baseline angiogram can be obtained, for example in the anterior/posterior (AP) and/or lateral views, prior to device insertion to assess occlusion location by injection of contrast media through the sheath 400 with fluoroscopic visualization. Fluoroscopic visualization may continue as the catheter system is advanced and subsequent angiograms can be captured periodically to assess reperfusion. The baseline angiogram image can be superimposed, such as with digital subtraction angiography, so that the vasculature and/or treatment site are visible while the catheter system is advanced.

Once the catheter system 150 is advanced into position (the positioning will be described in more detail below), the catheter advancement element 300 can be withdrawn and removed from the system. In some implementations, the catheter 200 can be used as a support catheter to deliver a stent or flow diverter (or implant delivery system) to the treatment site (e.g., within the carotid or a cerebral artery or a venous sinus or vein) as will be described elsewhere herein.

In an implementation, the guide sheath 400 includes one or more radiopaque markers 411. The radiopaque markers 411 can be disposed near the distal end opening 408. For example, a pair of radiopaque bands may be provided. The radiopaque markers 411 or markers of any of the system components can be swaged, painted, embedded, or otherwise disposed in or on the body. In some implementations, the radiopaque markers include a barium polymer, tungsten polymer blend, tungsten-filled or platinum-filled marker that maintains flexibility of the devices and improves transition along the length of the component and its resistance to kinking. In some implementations, the radiopaque markers are a tungsten-loaded PEBAX or polyurethane that is heat welded to the component.

The guide sheath markers 411 are shown in the figures as rings around a circumference of one or more regions of the body 402. However, the markers 411 can have other shapes or create a variety of patterns that provide orientation to an operator regarding the position of the distal opening 408 within the vessel. Accordingly, an operator may visualize a location of the distal opening 408 under fluoroscopy to confirm that the distal opening 408 is directed toward a target anatomy where a catheter 200 is to be delivered. For example, radiopaque marker(s) 411 allow an operator to rotate the body 402 of the guide sheath 400 at an anatomical access point, e.g., a groin of a patient, such that the distal opening provides access to an ICA by subsequent working device(s), e.g., catheters and wires advanced to the ICA. The access point need not only includes the femoral artery or femoral vein, but can also include sites such as radial, ulnar, or brachial arteries and veins, as well as a direct puncture of the carotid artery or jugular vein. In some implementations, the radiopaque marker(s) 411 include platinum, gold, tantalum, tungsten or any other substance visible under an x-ray fluoroscope. Any of the various components of the systems described herein can incorporate radiopaque markers.

Still with respect to FIGS. 7A-7B, the catheter 200 can include a relatively flexible, distal luminal portion 222 coupled to a stiffer, kink-resistant proximal extension or proximal control element 230. The term “control element” as used herein can refer to a proximal region configured for a user to cause pushing movement in a distal direction as well as pulling movement in a proximal direction. The control elements described herein may also be referred to as spines, tethers, push wires, push tubes, or other elements having any of a variety of configurations. The proximal control element 230 can be a hollow or tubular element. The proximal control element 230 can also be solid and have no inner lumen, such as a solid rod, ribbon or other solid wire type element. Generally, the proximal control elements described herein are configured to move its respective component (to which it may be attached or integral) in a bidirectional manner through a lumen.

A single, inner lumen 223 extends through the luminal portion 222 between a proximal end and a distal end of the luminal portion 222 (the lumen 223 is visible in FIG. 7B). In some implementations, a proximal opening 242 into the lumen 223 can be located near where the proximal control element 230 couples with the distal luminal portion 222. In other implementations, the proximal opening 242 into the lumen 223 is at a proximal end region of the catheter 200. A distal opening from the lumen 223 can be located near or at the distal-most end 215 of the luminal portion 222. The inner lumen 223 of the catheter 200 can have a first inner diameter and the working lumen of the guide sheath 400 can have a second, larger inner diameter. Upon insertion of the catheter 200 through the working lumen of the sheath 400, the lumen 223 of the catheter 200 can be configured to be fluidly connected and contiguous with the working lumen of the sheath 400 such that fluid flow into and/or out of the catheter system 100 is possible, such as by applying suction from a vacuum source coupled to the catheter system 100 at a proximal end. The combination of sheath 400 and catheter 200 can be continuously in communication with the bloodstream at the proximal end with advancement and withdrawal of catheter 200.

The distal luminal portion 222 of the catheter 200 can have one or more radiopaque markers 224. A first radiopaque marker 224a can be located near the distal-most end 215 to aid in navigation and proper positioning of the distal-most end 215 under fluoroscopy. Additionally, a proximal region of the catheter 200 may have one or more proximal radiopaque markers 224b so that the overlap region 348 can be visualized as the relationship between a radiopaque marker 411 on the guide sheath 400 and the radiopaque marker 224b on the catheter 200. The proximal region of the catheter 200 may also have one or more radiopaque markings providing visualization, for example, near the proximal opening 242 into the single lumen 223 of the catheter 200 as will be described in more detail below. In an implementation, the two radiopaque markers (marker 224a near the distal-most end 215 and a more proximal marker 224b) are distinct to minimize confusion of the fluoroscopic image, for example the catheter proximal marker 224b may be a single band and the marker 411 on the guide sheath 400 may be a double band and any markers on a working device delivered through the distal access system can have another type of band or mark. The radiopaque markers 224 of the distal luminal portion 222, particularly those near the distal end region navigating extremely tortuous anatomy, can be relatively flexible such that they do not affect the overall flexibility of the distal luminal portion 222 near the distal end region. The radiopaque markers 224 can be tungsten-loaded or platinum-loaded markers that are relatively flexible compared to other types of radiopaque markers used in devices where flexibility is not paramount. In some implementations, the radiopaque marker can be a band of tungsten-loaded PEBAX having a durometer of Shore A 35D.

The proximal control element 230 can include one or more markers 232 to indicate the overlap between the distal luminal portion 222 of the catheter 200 and the sheath body 402 as well as the overlap between the distal luminal portion 222 of the catheter 200 and other interventional devices that may extend through the distal luminal portion 222. At least a first mark can be an RHV proximity marker positioned so that when the mark is aligned with the sheath proximal hemostasis valve 434 during insertion of the catheter 200 through the guide sheath 400, the catheter 200 is positioned at the distal-most position with the minimal overlap length needed to create the seal between the catheter 200 and the working lumen. At least a second marker 232 can be a Fluoro-saver marker that can be positioned on the control element 230 and located a distance away from the distal-most end 215 of the distal luminal portion 222. In some implementations, a marker 232 can be positioned about 100 cm away from the distal-most end 215 of the distal luminal portion 222. The markers 232 can be positioned on the catheter so that one or more markers are visible to an operator outside the patient (and outside the guide sheath 400) during use. One or more markers can also be visible to an operator inside the patient (and inside the guide sheath 400 or beyond a distal end of the guide sheath 400) during use such that they are visualized under fluoroscopy.

The catheter 200 shown in FIGS. 7A-7B is less than full-length and includes a rapid exchange opening 242 into the distal luminal portion 222. The catheter 200 can incorporate a hub that is positioned relative to the proximal rapid exchange opening 242 a distance to prevent over-insertion of the catheter 200 relative to the sheath so the opening 242 stays inside the sheath during use. The hub can be sized to avoid entering the proximal valve component on the sheath so that the catheter 200 “bottoms out” before the opening 242 exits the distal opening of the sheath. This feature provides a passive mechanism to ensure an overlap sufficient for scaling (e.g., against a vacuum pressure of 300 mmHg up to about 700 mmHg) remains at all degrees of extension between the catheter 200 and the sheath and the opening 242 stays inside the sheath lumen, even when the catheter 200 extends a maximum distance beyond the distal end of the sheath, such as to reach distal intracranial vessels. Similarly, when nested catheters 200 are used, each catheter 200 can have a length such that the proximal hub is positioned to ensure the hub of the respective catheter 200 bottoms out and prevents the respective opening 242 from exiting the distal opening of the catheter is extends distal to. The catheter 200 can also be a full-length catheter having a lumen that ends between the distal and proximal opening, the proximal opening configured to remain outside the sheath hub and outside the patient.

Still with respect to FIGS. 7A-7B and also FIG. 8A, the catheter advancement element 300 can include a non-expandable, flexible elongate body 360 (sometimes referred to herein as a tubular portion) and a proximal portion 366. The catheter advancement element 300 and the catheter 200 described herein may be configured for rapid exchange or over-the-wire methods. For example, the flexible elongate body 360 can be a tubular portion extending the entire length of the catheter advancement element 300 and can have a proximal opening from the lumen 368 of the flexible elongate body 360 that is configured to extend outside the patient's body during use. Alternatively, the tubular portion can have a proximal opening positioned such that the proximal opening remains inside the patient's body during use. The proximal portion 366 can be a proximal element coupled to a distal tubular portion 360 and extending proximally therefrom. A proximal opening from the tubular portion 360 can be positioned near where the proximal element 366 couples to the tubular portion 360. Alternatively, the proximal portion 366 can be an extension of the tubular portion 360 having a length that extends to a proximal opening near a proximal terminus of the catheter advancement element 300 (i.e., outside a patient's body). A luer 364 can be coupled to the proximal portion 366 at the proximal end region so that tools, such as a guidewire, can be advanced through the lumen 368 of the catheter advancement element 300. A syringe or other component can be coupled to the luer 364 in order to draw a vacuum and/or inject fluids through the lumen 368. The syringe coupled to the luer 364 can also be used to close off the lumen of the catheter advancement element 300 to maximize the piston effect described elsewhere herein.

The configuration of the proximal portion 366 can vary. In some implementations, the proximal portion 366 is simply a proximal extension of the flexible elongate body 360 that does not change significantly in structure but changes in flexibility. For example, the proximal portion 366 transitions from the very flexible distal regions of the catheter advancement element 300 towards less flexible proximal regions of the catheter advancement element 300. In some implementations, the proximal portion 366 can provide a relatively stiff proximal end suitable for manipulating (e.g., advancing and withdrawing) the more distal regions of the catheter advancement element 300 relative to the anatomy and/or the outer catheter 200. The proximal portion 366 can be formed of a less flexible polymer than the flexible elongate body. The proximal portion 366 can be fully polymeric having no reinforcement or the proximal portion 366 can be a reinforced polymer portion. The configuration of the proximal portion 366 can vary depending on whether the catheter advancement element 300 is to be used with a full-length catheter or a catheter having only a short distal tubular portion. The catheter advancement element 300 used with a full-length catheter need not rely upon a proximal reinforcement in order to advance the catheter system through the anatomy and can instead rely on the proximal stiffness of the outer catheter. A catheter advancement element 300 used with a partial tube outer catheter may benefit from a stiffer reinforcement within its proximal end region for advancing the system.

In some implementations, the proximal portion 366 is a metal reinforced segment. The metal reinforced segment can be positioned a distance away from the distal end of the elongate body. For example, the metal reinforced segment can be about 50 cm from the distal end. The metal reinforced segment can have an inner diameter of about 0.021″ and an outer diameter of about 0.027″. The metal reinforced segment can be a spine. The metal reinforced segment can be a hypotube. In other implementations, the proximal portion 366 is a hypotube. The hypotube may be exposed or may be coated by a polymer. In still further implementations, the proximal portion 366 may be a tubular polymer portion reinforced by a coiled ribbon or braid. The proximal portion 366 can have the same outer diameter as the flexible elongate body or can have a smaller outer diameter as the flexible elongate body.

The proximal portion 366 need not include a lumen. For example, the proximal portion 366 can be a solid rod, ribbon, or wire have no lumen extending through it that couples to the tubular elongate body 360. Where the proximal portion 366 is described herein as having a lumen, it should be appreciated that the proximal portion 366 can also be solid and have no lumen. The proximal portion 366 is generally less flexible than the elongate body 360 and can transition to be even more stiff towards the proximal-most end of the proximal portion 366. Thus, the catheter advancement element 300 can have an extremely soft and flexible distal end region 346 that transitions proximally to a stiff proximal portion 366 well suited for pushing and/or torquing the distal elongate body 360.

The elongate body 360 can be received within and extended through the internal lumen 223 of the distal luminal portion 222 of the catheter 200 (see FIG. 2B). The elongate body 360 or tubular portion can have an outer diameter. The outer diameter of the tubular portion can have at least one snug point. The at least one snug point provides a close fit between the elongate body 360 and the distal luminal portion 222 that minimizes a distal lip or edge at the distal end of the catheter 200, but that still allows for movement relative to one another so as to allow a user to achieve a desired extension or withdrawal of the catheter advancement element 300 relative to the catheter 200 or the catheter 200 relative to the catheter advancement element 300. The snug point allows for movement between the catheters upon application of a relatively small load so as to avoid any negative impact on usability within a patient. A difference between the inner diameter of the catheter 200 and the outer diameter of the tubular portion at the snug point can be no more than about 0.015″ (0.381 mm), or can be no more than about 0.010″ (0.254 mm), for example, from about 0.003″ (0.0762 mm) up to about 0.012″ (0.3048 mm), preferably about 0.005″ (0.127 mm) to about 0.010″ (0.254 mm), and more preferably about 0.007″ (0.1778 mm) to about 0.009″ (0.2286 mm).

As will be described in more detail below, the catheter advancement element 300 can also include a distal end region 346 located distal to the at least one snug point of the tubular portion. The distal end region 346 can have a length and taper along at least a portion of the length. The distal end region 346 of the catheter advancement element 300 can be extended beyond the distal end of the catheter 200 as shown in FIG. 7B. The proximal portion 366 of the catheter advancement element 300 or proximal extension is coupled to a proximal end region of the elongate body 360 and extends proximally therefrom. The proximal portion 366 can be less flexible than the elongate body 360 and configured for bi-directional movement of the elongate body 360 of the catheter advancement element 300 within the luminal portion 222 of the catheter 200, as well as for movement of the catheter system 100 as a whole. The elongate body 360 can be inserted in a coaxial fashion through the internal lumen 223 of the luminal portion 222. The outer diameter of at least a region of the elongate body 360 can be sized to substantially fill at least a portion of the internal lumen 223 of the luminal portion 222.

The overall length of the catheter advancement element 300 (e.g., between the proximal end through to the distal-most tip) can vary, but generally is long enough to extend through the support catheter 200 plus at least a distance beyond the distal end of the support catheter 200 while at least a length of the proximal portion 366 remains outside the proximal end of the guide sheath 400 and outside the body of the patient. In some implementations, the overall length of the catheter advancement element 300 is about 145 to about 150 cm and has a working length of about 140 cm to about 145 cm from a proximal tab or hub to the distal-most end 325. The elongate body 360 can have a length that is at least as long as the luminal portion 222 of the catheter 200 although the elongate body 360 can be shorter than the luminal portion 222 so long as at least a minimum length remains inside the luminal portion 222 when a distal portion of the elongate body 360 is extended distal to the distal end of the luminal portion 222 to form a snug point or snug region with the catheter. In some implementations, this minimum length of the elongate body 360 that remains inside the luminal portion 222 when the distal end region 346 is positioned at its optimal advancement configuration is at least about 5 cm, at least about 6 cm, at least about 7 cm, at least about 8 cm, at least about 9 cm, at least about 10 cm, at least about 11 cm, or at least about 12 cm up to about 50 cm. In some implementations, the shaft length of the distal luminal portion 222 can be about 35 cm up to about 75 cm and shorter than a working length of the guide sheath and the insert length of the elongate body 360 can be at least about 45 cm, 46 cm, 47 cm, 48 cm, 48.5 cm, 49 cm, 49.5 cm up to about 85 cm.

The length of the elongate body 360 can allow for the distal end of the elongate body 360 to reach cerebrovascular targets or occlusions within, for example, segments of the internal carotid artery including the cervical (C1), petrous (C2), lacerum (C3), cavernous (C4), clinoid (C5), ophthalmic (C6), and communicating (C7) segments of the internal carotid artery (ICA) as well as branches off these segments including the M1 or M2 segments of the middle cerebral artery (MCA), anterior cerebral artery (ACA), anterior temporal branch (ATB), and/or posterior cerebral artery (PCA). The distal end region of the elongate body 360 can reach these distal target locations while the proximal end region of the elongate body 360 remains proximal to or below the level of severe turns along the path of insertion. For example, the entry location of the catheter system can be in the femoral artery (or radial, ulnar, or brachial arteries, as well as direct puncture of the carotid artery) and the target occlusion location can be distal to the right common carotid artery, such as within the M1 segment of the middle cerebral artery on the right side. The proximal end region of the elongate body 360 where it transitions to the proximal portion 366 can remain within a vessel that is proximal to severely tortuous anatomy, such as the carotid siphon, the right common carotid artery, the brachiocephalic trunk, the take-off into the brachiocephalic artery from the aortic arch, the aortic arch as it transitions from the descending aorta. This avoids inserting the stiffer proximal portion 366, or the material transition between the stiffer proximal portion 366 and the elongate body 360, from taking the turn of the aortic arch or the turn of the brachiocephalic take-off from the aortic arch, which both can be very severe. The lengths described herein for the distal luminal portion 222 also can apply to the elongate body 360 of the catheter advancement element.

The proximal portion 366 can have a length that varies as well. In some implementations, the proximal portion 366 is about 90 cm up to about 95 cm. The distal portion extending distal to the distal end of the luminal portion 222 can include distal end region 346 that protrudes a length beyond the distal end of the luminal portion 222 during use of the catheter advancement element 300. The distal end region 346 of the elongate body 360 that is configured to protrude distally from the distal end of the luminal portion 222 during advancement of the catheter 200 through the tortuous anatomy of the cerebral vessels, as will be described in more detail below. The proximal portion 366 coupled to and extending proximally from the elongate body 360 can align generally side-by-side with the proximal control element 230 of the catheter 200. The arrangement between the elongate body 360 and the luminal portion 222 can be maintained during advancement of the catheter 200 through the tortuous anatomy to reach the target location for treatment in the distal vessels and aids in preventing the distal end of the catheter 200 from catching on tortuous branching vessels, as will be described in more detail below.

In some implementations, the elongate body 360 can have a region of relatively uniform outer diameter extending along at least a portion of its length and the distal end region 346 tapers down from the uniform outer diameter. The outer diameter of the elongate body 360 also can taper or step down in outer diameter proximally, for example near where the elongate body 360 couples or transitions to the proximal portion 366. The outer diameter of the elongate body 360 need not change in outer diameter near where the elongate body 360 couples or transitions to the proximal portion 366. In some implementations, the region of relatively uniform outer diameter can extend along a majority of the working length of the catheter advancement element 300 including the proximal portion 366. This first region of uniform outer diameter can transition to a second region of uniform outer diameter located distal to the first region. The transition can incorporate a smooth taper or step change in outer diameter between the two regions. The second region of uniform outer diameter having the larger size and located distal to the first region can be useful in filling a lumen of a larger bore catheter without the entire working length of the elongate body needing to have this larger size. In this implementation, the elongate body 360 can have a distal taper changing in diameter from the second uniform diameter region towards the distal opening and a proximal taper changing in diameter from the second uniform diameter region towards the first region of uniform outer diameter.

Depending upon the inner diameter of the catheter 200, the difference between the inner diameter of catheter 200 and the outer diameter of the elongate body 360 along at least a portion of its length, such as at least 10 cm of its length, preferably at least 15 cm of its length can be no more than about 0.015″ (0.381 mm), such as within a range of about 0.003″-0.015″ (0.0762 mm-0.381 mm) or between 0.006″-0.010″ (0.1524 mm-0.254 mm). Thus, the clearance between the catheter 200 and the elongate body 360 can result in a space on opposite sides that is no more than about 0.008″ (0.2032 mm), or can be no more than about 0.005″ (0.127 mm), for example, from about 0.001″ up to about 0.006″ (0.0254 mm-0.1524 mm), preferably about 0.002″ to about 0.005″ (0.0508 mm-0.127 mm), and more preferably about 0.003″ to about 0.005″ (0.0762 mm-0.0508 mm).

The catheter advancement element 300 has a large outer diameter and a relatively small inner diameter, particularly when a guidewire extends into or through the lumen of the catheter advancement element 300. The elongate body 360 can have an overall shape profile from proximal end to distal end that transitions from a first outer diameter having a first length to a tapering outer diameter having a second length. The first length of this first outer diameter region (i.e., the snug-fitting region between the distal luminal portion 222 and the elongate body 360) can be at least about 5 cm, or 10 cm, up to about 50 cm, including 5, 6, 7, 8, 9, 10, 11, 12, 13, 14, 15, 16, 17, 18, 19, 20, 21, 22, 23, 24, 25, 30, 35, 40, 45, up to about 50 cm and any length in between. In other implementations, the snug-fitting region can extend from the proximal tab or luer 364 substantially to the tapered distal end region 346 which depending on the length of the catheter advancement element 300, can be up to about 170 cm. The length of the tapering outer diameter of the distal end region 346 can be about 0.5 cm to about 5 cm, about 1 cm to about 4 cm, or about 1.5 cm to about 3 cm, or between 2.0 cm and about 2.5 cm. In some implementations, the length of the distal end region 346 varies depending on the inner diameter of the catheter 200 with which the catheter advancement element 300 is to be used. For example, the length of the distal end region 346 can be shorter (e.g., 1.2 cm) for a catheter advancement element 300 sized to be used with a catheter 200 having an inner diameter of about 0.054″ (1.372 mm) and can be longer (e.g., 2.5 cm) for a catheter advancement element 300 sized to be used with a catheter 200 having an inner diameter of about 0.088″ (2.235 mm). The distal end region 346 can be a constant taper from the larger outer diameter of the elongate body 360 (e.g., the distal end of the marker 344b) down to a second smaller outer diameter at the distal-most terminus (e.g., the proximal end of the marker 344a) as shown in FIG. 8A. In some implementations, the constant taper of the distal end region 346 can be from about 0.048″ outer diameter down to about 0.031″ (0.787 mm) outer diameter over a length of about 1 cm. In some implementations, the constant taper of the distal end region 346 can be from 0.062″ (1.575 mm) outer diameter to about 0.031″ (0.787 mm) outer diameter over a length of about 2 cm. In still further implementations, the constant taper of the distal end region 346 can be from 0.080″ (2.032 mm) outer diameter to about 0.031″ (0.787 mm) outer diameter over a length of about 2.5 cm. The length of the constant taper of the distal end region 346 can vary, for example, between 0.8 cm to about 2.5 cm, or between 1 cm and 3 cm, or between 2.0 cm and 2.5 cm. The angle of the taper can vary depending on the outer diameter of the elongate body 360. For example, the angle of the taper can be between 0.9 to 1.6 degrees relative to horizontal. The angle of the taper can be between 2-3 degrees from a center line of the elongate body 360. The length of the taper of the distal end region 346 can be between about 5 mm to 20 mm or about 20 mm to about 50 mm.

The distal end region 346 of the elongate body 360 can also be shaped with or without a taper. When the catheter advancement element 300 is inserted through the catheter 200, this distal end region 346 is configured to extend beyond and protrude out through the distal-most end 215 of the luminal portion 222 whereas the more proximal region of the body 360 (i.e., the first length described above) remains within the luminal portion 222.

As mentioned, the distal-most end 215 of the luminal portion 222 can be blunt and have no change in the dimension of the outer diameter whereas the distal end region 346 can be tapered providing an overall elongated tapered geometry of the catheter system. The outer diameter of the elongate body 360 also approaches the inner diameter of the luminal portion 222 such that the step-up from the elongate body 360 to the outer diameter of the luminal portion 222 is minimized. Minimizing this step-up prevents issues with the lip formed by the distal end of the luminal portion 222 catching on the tortuous neurovasculature, such as around the carotid siphon near the ophthalmic artery branch, when the distal end region 346 in combination with the distal end region of the catheter 200 bends and curves along within the vascular anatomy. In some implementations, the inner diameter of the luminal portion 222 can be at least about 0.052″ (1.321 mm), about 0.054″ (1.372 mm) and the maximum outer diameter of the elongate body 360 can be about 0.048″ (1.219 mm) such that the difference between them is about 0.006″ (0.1524 mm). In some implementations, the inner diameter of the luminal portion 222 can be about 0.070″ (1.778 mm) and the maximum outer diameter of the elongate body 360 can be about 0.062″ (1.575 mm) such that the difference between them is about 0.008″ (0.2032 mm). In some implementations, the inner diameter of the luminal portion 222 can be about 0.088″ (2.235 mm) and the maximum outer diameter of the elongate body 360 can be about 0.080″ (2.032 mm) such that the difference between them is about 0.008″ (0.2032 mm). In some implementations, the inner diameter of the luminal portion 222 can be about 0.072″ (1.829 mm) and the maximum outer diameter of the elongate body 360 is about 0.070″ (1.778 mm) such that the difference between them is only 2 thousandths of an inch (0.002″/0.0508 mm). In other implementations, the maximum outer diameter of the elongate body 360 is about 0.062″ (1.575 mm) such that the difference between them is about 0.010″ (0.254 mm). Despite the outer diameter of the elongate body 360 extending through the lumen of the luminal portion 222, the luminal portion 222 and the elongate body 360 extending through it in co-axial fashion are flexible enough to navigate the tortuous anatomy leading to the level of M1 or M2 arteries without kinking and without damaging the vessel. It is preferred to deliver a catheter that is as large in inner diameter for the passage of larger-sized implant delivery systems.

The dimensions provided herein are approximate and each dimensions may have an engineering tolerance or a permissible limit of variation. Use of the term “about,” “approximately,” or “substantially” are intended to provide such permissible tolerance to the dimension being referred to. Where “about” or “approximately” or “substantially” is not used with a particular dimension herein that that dimension need not be exact.

The elongate body 360 of the catheter advancement element 300 can have a lumen 368 with an inner diameter that does not change over the length of the elongate body even in the presence of the tapering of the distal end region 346. Thus, the inner diameter of the lumen 368 extending through the tubular portion of the catheter advancement element 300 can remain uniform and the wall thickness of the distal end region 346 can decrease to provide the taper. The wall thickness can thin distally along the length of the taper. Thus, the material properties in combination with wall thickness, angle, length of the taper can all contribute to the overall maximum flexibility of the distal-most end of the distal end region 346. The catheter advancement element 300 undergoes a transition in flexibility from the distal-most end towards the snug point where it achieves an outer diameter that is no more than about 0.010″ (0.254 mm) different from the inner diameter of the catheter 200.

The inner diameter of the elongate body 360 can be constant along its length even where the single lumen passes through the tapering distal end region 346. Alternatively, the inner diameter of the elongate body 360 can have a first size through the tapering distal end region 346 and a second, larger size through the cylindrical section of the elongate body 360. The cylindrical section of the elongate body 360 can have a constant wall thickness or a wall thickness that varies to a change in inner diameter of the cylindrical section. As an example, the outer diameter of the cylindrical section of the elongate body 360 can be about 0.080″. The inner diameter of the elongate body 360 within the cylindrical section can be uniform along the length of the cylindrical section and can be about 0.019″. The wall thickness in this section, in turn, can be about 0.061″. As another example, the outer diameter of the cylindrical section of the elongate body 360 can again be between about 0.080″. The inner diameter of the elongate body 360 within the cylindrical section can be non-uniform along the length of the cylindrical section and can step-up from a first inner diameter of about 0.019″ to a larger second inner diameter of about 0.021″. The wall thickness, in turn, can be about 0.061″ at the first inner diameter region and about 0.059″ at the second inner diameter region. The wall thickness of the cylindrical portion of the elongate body 360 can be between about 0.050″ to about 0.065″. The wall thickness of the tapered distal end region 346 near the location of the proximal marker band can be the same as the cylindrical portion (between about 0.050″ and about 0.065″) and become thinner towards the location of the distal marker band. As an example, the inner diameter at the distal opening from the single lumen can be about 0.020″ and the outer diameter at the distal opening (i.e., the outer diameter of the distal marker band) and be about 0.030″ resulting in a wall thickness of about 0.010″ compared to the wall thickness of the cylindrical portion that can be up to about 0.065″. Thus, the outer diameter of the distal tip 346 can taper as can the wall thickness. A wall thickness of the intermediate segment and an un-tapered portion of the tip segment can be about 0.050″ to about 0.065″. The wall thickness of the intermediate segment and the un-tapered portion can be constant. The inner diameter of the intermediate segment and the tapered end region can be constant.

A tip segment of the flexible elongate body can have a tapered portion that tapers distally from a first outer diameter to a second outer diameter. The second outer diameter can be about ½ of the first outer diameter. The second outer diameter can be about 40% of the first outer diameter. The second outer diameter can be about 65% of the first outer diameter. The first outer diameter can be about 0.062″ up to about 0.080″. The second outer diameter can be about 0.031″. The second outer diameter can be about 50% of the first outer diameter, about 40% of the first outer diameter, or about 65% of the first outer diameter.

The length of the taper can also vary depending on the anatomy of the target region. The distal end region 346 can achieve its soft, atraumatic and flexible characteristic due to a material property other than due to a change in outer dimension to facilitate endovascular navigation to an occlusion in tortuous anatomy. Additionally or alternatively, the distal end region 346 of the elongate body 360 can have a transition in flexibility along its length. The most flexible region of the distal end region 346 can be its distal terminus. Moving along the length of the distal end region 346 from the distal terminus towards a region proximal to the distal terminus. For example, the distal end region 346 can be formed of a material having a Shore material hardness of no more than 35D or about 62A and transitions proximally to be less flexible near where it is formed of a material having a material hardness of no more than 55D and 72D up to the proximal portion 366, which can be a stainless steel hypotube, or a combination of a material property and tapered shape. The materials used to form the regions of the elongate body 360 can include PEBAX (such as PEBAX 25D, 35D, 55D, 69D, 72D) or a blend of PEBAX (such as a mix of 25D and 35D, 25D and 55D, 25D and 72D, 35D and 55D, 35D and 72D, 55D and 72D, where the blend ratios may range from 0.1% up to 50% for each PEBAX durometer), with a lubricious additive compound, such as Mobilize (Compounding Solutions, Lewiston, Maine). In some implementations, the material used to form a region of the elongate body 360 can be Tecothane 62A. Incorporation of a lubricious additive directly into the polymer elongate body means incorporation of a separate lubricious liner, such as a Teflon liner, is unnecessary. This allows for a more flexible element that can navigate the distal cerebral anatomy and is less likely to kink. Similar materials can be used for forming the distal luminal portion 222 of the catheter 200 providing similar advantages. The flexibility of the distal end region 346 can be achieved by a combination of flexible lubricious materials and tapered shapes. For example, the length of the distal end region 346 can be kept shorter than 2 cm-3 cm, but maintain optimum deliverability due to a change in flexible material from distal-most end 325 towards a more proximal region a distance away from the distal-most end 325. In an implementation, the elongate body 360 is formed of PEBAX (polyether block amide) embedded silicone designed to maintain the highest degree of flexibility. The wall thickness of the distal end of the luminal portion 222 can also be made thin enough such that the lip formed by the distal end of the luminal portion 222 relative to the elongate body 360 is minimized.

The elongate body 360 has a benefit over a microcatheter in that it can have a relatively large outer diameter that is just 0.003″-0.010″ (0.0762 mm-0.254 mm) smaller than the inner diameter of the distal luminal portion 222 of the catheter 200 and still maintain a high degree of flexibility for navigating tortuous anatomy. When the gap between the two components is too tight (e.g., less than about 0.003″ (.0762 mm), the force needed to slide the catheter advancement element 300 relative to the catheter 200 can result in damage to one or both of the components and increases risk to the patient during the procedure. The gap results in too tight of a fit to provide optimum relative sliding. When the gap between the two components is too loose (e.g., greater than about 0.010″/0.254 mm), the distal end of the catheter 200 forms a lip that is prone to catch on carotid dissections or branching vessels during advancement through tortuous neurovasculature, such as around the carotid siphon where the ophthalmic artery branches off and the piston effect of withdrawal of the elongate body 360 can be decreased or lost.

The gap in ID/OD between the elongate body 360 and the distal luminal portion 222 can be in this size range (e.g., 0.003″-0.015″ (0.0762 mm-0.381 mm) or between 0.006″-0.010″ (0.152 mm-0.254 mm)) along a majority of their lengths. For example, the elongate body 360 can have a relatively uniform outer diameter that is between about 0.048″ (1.219 mm) to about 0.080″ (2.032 mm) from a proximal end region to a distal end region up to a point where the taper of the distal end region 346 begins. Similarly, the distal luminal portion 222 of the catheter 200 can have a relatively uniform inner diameter that is between about 0.054″ (1.372 mm) to about 0.088″ (2.235 mm) from a proximal end region to a distal end region. As such, the difference between their respective inner and outer diameters along a majority of their lengths can be within this gap size range of 0.003″ to 0.015″ (0.0762 mm-0.381 mm). The distal end region 346 of the elongate body 360 that is tapered will have a larger gap size relative to the inner diameter of the distal luminal portion 222. During use, however, this tapered distal end region 346 is configured to extend distal to the distal end of the catheter 200 such that the region of the elongate body 360 having an outer diameter sized to match the inner diameter of the distal luminal portion 222 is positioned within the lumen of the catheter 200 such that it can minimize the lip at the distal end of the catheter 200.

The elongate body 360 can be formed of various materials that provide a suitable flexibility and lubricity. Example materials include high density polyethylene, 77A PEBAX, 33D PEBAX, 42D PEBAX, 46D PEBAX, 54D PEBAX, 69D PEBAX, 72D PEBAX, 90D PEBAX, and mixtures thereof or equivalent stiffness and lubricity material. In some implementations, the elongate body 360 is an unreinforced, non-torquing catheter having a relatively large outer diameter designed to fill the lumen it is inserted through and a relatively small inner diameter to minimize any gaps at a distal-facing end of the device. In other implementations, at least a portion of the elongate body 360 can be reinforced to improve navigation and torquing (e.g., braided reinforcement layer). The flexibility of the elongate body 360 can increase towards the distal end region 346 such that the distal region of the elongate body 360 is softer, more flexible, and articulates and bends more easily than a more proximal region. For example, a more proximal region of the elongate body can have a bending stiffness that is flexible enough to navigate tortuous anatomy, such as the carotid siphon, without kinking. If the elongate body 360 has a braid reinforcement layer along at least a portion of its length, the braid reinforcement layer can terminate a distance proximal to the distal end region 346. For example, the distance from the end of the braid to the distal-most end 325 can be about 10 cm to about 15 cm or from about 4 cm to about 10 cm or from about 4 cm up to about 15 cm.

In some implementations, the elongate body 360 can be generally tubular along at least a portion of its length such that it has a single lumen 368 extending parallel to a longitudinal axis of the catheter advancement element 300 (see FIGS. 7A-7B and 8A-8B). In an implementation, the single lumen 368 of the elongate body 360 is sized to accommodate a guidewire, however use of the catheter advancement element 300 generally eliminates the need for a guidewire lead. Preferably, the assembled system includes no guidewire, or a guidewire parked inside the lumen 368 retracted away from the distal opening. Guidewires are designed to be exceptionally flexible so that they deflect to navigate the severe turns of the anatomy. However, many workhorse guidewires have a stiffness along their longitudinal axis and/or are small enough in outer diameter that they find their own paths through an occlusion rather than slipping around the occlusion or get hung up on vessel wall dissections increasing the risk of perforations. In some cases, these guidewires can cause perforations and/or dissections of the vessel itself. Guidewires tend to get redirected into branches rather than remaining within the larger vessel. This makes them helpful for selecting a branch, but problematic for navigating tortuous anatomy and following the main flow of blood. Thus, even though the guidewire may have an outer diameter at its distal tip region that is small and very flexible at the distal tip, guidewires typically are incapable of atraumatically probing an occlusion or other structure such that the pose a risk of perforation with repeated advancement. Guidewires do not deflect upon encountering something relatively dense, such as the proximal face of the occlusion or a dissection flap. Instead, guidewires embed and penetrate such structures. The catheter advancement element 300 has a softness, taper, and sizing that finds and/or creates space. For example, the catheter advancement element 300 upon encountering an occlusion, such as an atherosclerotic lesion or embolus, can slide between a portion of the occlusion and the vessel wall rather than penetrating through it like a guidewire does. In the case of a partially occluded vessel, such as a narrowing within the carotid artery, the catheter advancement element 300 can atraumatically and safely find the path through the narrowing. The catheter advancement element 300 also deflects away from a dissection flap so as to remain within the larger lumen. The softness, taper, and sizing of the catheter advancement element 300 allows for it to be repeatedly passed through the carotid and into the cerebral arteries without penetrating or taking a detour relative to these structures. The distal tip region deflects and passes by these structures so that the catheter system is advanced past them to a distal occlusion site or probes and wedges near them in a safe manner.

A guidewire 500 can extend through the single lumen 368 generally concentrically from a proximal opening to a distal opening 326 at the distal-most end 325 of the catheter advancement element 300 (see FIG. 8B). In some implementations, the proximal opening is at the proximal end of the catheter advancement element 300 such that the catheter advancement element 300 is configured for over-the-wire (OTW) methodologies. In other implementations, the proximal opening is a rapid exchange opening through a wall of the catheter advancement element 300 such that the catheter advancement element 300 is configured for rapid exchange rather than or in addition to OTW. In this implementation, the proximal opening extends through the sidewall of the elongate body 360 and is located a distance away from a proximal tab or luer 364 and distal to the proximal portion 366. The proximal opening can be located a distance of about 10 cm from the distal end region 346 up to about 20 cm from the distal end region 346. In some implementations, the proximal opening can be located near a region where the elongate body 360 is joined to the proximal portion 366, for example, just distal to an end of the hypotube. In other implementations, the proximal opening is located more distally, such as about 10 cm to about 18 cm from the distal-most end of the elongate body 360. A proximal opening that is located closer to the distal end region 346 allows for easier removal of the catheter advancement element 300 from the catheter 200 leaving the guidewire in place for a “rapid exchange” type of procedure. Rapid exchanges can rely on only a single person to perform the exchange. The catheter advancement element 300 can be readily substituted for another device using the same guidewire that remains in position. The single lumen 368 of the elongate body 360 can be configured to receive a guidewire 500 in the range of 0.014″ (0.356 mm) and 0.018″ (0.457 mm) diameter, or in the range of between 0.014″ and 0.022″ (0.356 mm-0.559 mm). In this implementation, the inner luminal diameter of the elongate body 360 can be between 0.020″ and 0.024″ (0.508 mm-0.610 mm). The guidewire, the catheter advancement element 300, and the catheter 200 can all be assembled co-axially for insertion through the working lumen of the guide sheath 400. The inner diameter of the lumen 368 of the elongate body 360 can be 0.019″ to about 0.021″ (0.483 mm-0.533 mm). The distal opening 326 from the lumen 368 can have an inner diameter that is between about 0.018″ to about 0.024″ (0.457 mm-0.610 mm). The distal opening 326 from the lumen 368 can have an inner diameter that is between about 0.016″ to about 0.028″. The distal opening 326 is sized to receive a guidewire that can be a 0.014″ to a 0.024″ guidewire.

The region near the distal end region 346 can be tapered such that the outer diameter tapers over a length of about 0.5 cm to about 5 cm, or 1 cm to about 4 cm, or other length as described elsewhere herein. The larger outer diameter can be at least about 1.5 times, 2 times, 2.5 times, or about 3 times larger than the smaller outer diameter. The distal end region 346 can taper along a distance from a first outer diameter to a second outer diameter, the first outer diameter being at least 1.5 times the second outer diameter. In some implementations, the distal end region 346 tapers from about 0.080″ (2.032 mm) to about 0.031″ (0.787 mm). In some implementations, the smaller outer diameter at a distal end of the taper can be about 0.026″ (0.66 mm) up to about 0.040″ (1.016 mm) and the larger outer diameter proximal to the taper is about 0.062″ (1.575 mm) up to about 0.080″ (2.032 mm). Also, the distal end region 346 can be formed of a material having a material hardness (e.g., 62A and 35D) that transitions proximally towards increasingly harder materials having (e.g., 55D and 72D) up to the proximal portion 366. A first segment of the elongate body 360 including the distal end region 346 can be formed of a material having a material hardness of 35D and a length of about 10 cm to about 12.5 cm. The first segment of the elongate body 360 including the distal end region 346 can be formed of a material having a material hardness of 62A and a length of about 10 cm to about 12.5 cm. A second segment of the elongate body 360 can be formed of a material having a material hardness of 55D and have a length of about 5 cm to about 8 cm. A third segment of the elongate body 360 can be formed of a material having a material hardness of 72D can be about 25 cm to about 35 cm in length. The three segments combined can form an insert length of the elongate body 360 from where the proximal portion 366 couples to the elongate body 360 to the terminus of the distal end region 346 that can be about 49 cm in length.

The catheter advancement element 300 can incorporate a reinforcement layer. The reinforcement layer can be a braid or other type of reinforcement to improve the torqueability of the catheter advancement element 300 and help to bridge the components of the catheter advancement element 300 having such differences in flexibility. The reinforcement layer can bridge the transition from the rigid, proximal portion 366 to the flexible elongate body 360. In some implementations, the reinforcement layer can be a braid positioned between inner and outer layers of PEBAX. The reinforcement layer can terminate a distance proximal to the distal end region 346. The distal end region 346 can be formed of a material having a material hardness of at most about 35D. The first segment can be unreinforced polymer having a length of about 4 cm up to about 12.5 cm without metal reinforcement. The third segment of the elongate body 360 located proximal to the first segment can include the reinforcement layer and can extend a total of about 37 cm up to the unreinforced distal segment. A proximal end region of the reinforcement layer can overlap with a distal end region of the proximal portion 366 such that a small overlap of hypotube and reinforcement exists near the transition between the proximal portion 366 and the elongate body 360.

The tubular portion of the catheter advancement element 300 can have an outer diameter that has at least one snug point. A difference between the outer diameter at the snug point and the inner diameter of the lumen at the distal end of the distal, catheter portion can be no more than about 0.015″ (0.381 mm), or can be no more than about 0.010″ (0.254 mm). The at least one snug point of this tubular portion can be a point along the length of the tubular portion. The at least one snug point of this tubular portion can have a length that is at least about 5 cm up to about 50 cm, including for example, at least about 6 cm, at least about 7 cm, at least about 8 cm, at least about 9 cm, at least about 10 cm, at least about 11 cm, or at least about 12 cm up to about 50 cm. This length need not be uniform such that the length need not be snug along its entire length. For example, the snug point region can include ridges, grooves, slits, or other surface features.

In other implementations, the entire catheter advancement element 300 can be a tubular element configured to receive a guidewire 500 through both the proximal portion 366 as well as the elongate body 360. For example, the proximal portion 366 can be a tubular element having a lumen that communicates with the lumen 368 extending through the elongate body 360. In some implementations, the proximal portion 366 can be a skived hypotube of stainless steel coated with PTFE having an outer diameter of 0.026″ (0.660 mm). In other implementations, the outer diameter can be between 0.024″ (0.610 mm) and 0.030″ (0.762 mm). In some implementations, such as an over-the-wire version, the proximal portion 366 can be a skived hypotube coupled to a proximal hub or luer 364. The proximal portion 366 can extend eccentric or concentric to the distal luminal portion 222. The proximal portion 366 can be a stainless steel hypotube. The proximal portion 366 can be a solid metal wire that is round or oval cross-sectional shape. The proximal portion 366 can be a flattened ribbon of wire having a rectangular cross-sectional shape. The ribbon of wire can be curved into a circular, oval, c-shape, or quarter circle, or other cross-sectional shape along an arc. The proximal portion 366 can have any of variety of cross-sectional shapes whether or not a lumen extends therethrough, including a circular, oval, C-shaped, D-shape, or other shape. In some implementations, the proximal portion 366 is a hypotube having a D-shape such that an inner-facing side is flat, and an outer-facing side is rounded. The rounded side of the proximal portion 366 can be shaped to engage with a correspondingly rounded inner surface of the sheath 400. The hypotube can have a lubricious coating, such as PTFE or another lubricious polymer covering the hypotube. The hypotube can have an inner diameter of about 0.021″ (0.533 mm), an outer diameter of about 0.0275″ (0.699 mm), and an overall length of about 94 cm providing a working length for the catheter advancement element 300 that is about 143 cm. Including the proximal luer 364, the catheter advancement element 300 can have an overall length of about 149 cm. In some implementations, the hypotube can be a tapered part with a length of about 100 mm, starting proximal with a thickness of 0.3 mm and ending with a thickness of 0.10 mm to 0.15 mm. In still further implementations, the elongate body 360 can be a solid element coupled to the proximal portion 366 having no guidewire lumen.

The proximal portion 366 is shown in FIG. 7A as having a smaller outer diameter compared to the outer diameter of the elongate body 360. The proximal portion 366 need not step down in outer diameter and can also have the same outer diameter as the outer diameter as the elongate body 360. The proximal portion 366 can incorporate a hypotube or other stiffening element that is coated by one or more layers of polymer resulting in a proximal portion 366 having substantially the same outer diameter as the elongate body 360.

At least a portion of the solid elongate body 360, such as the elongate distal end region 346, can be formed of or embedded with or attached to a malleable material that skives down to a smaller dimension at a distal end. The distal end region 346 can be shaped to a desired angle or shape similar to how a guidewire may be used. The malleable length of the elongate body 360 can be at least about 1 cm, 3 cm, 5 cm, and up to about 10 cm, 15 cm, or longer. In some implementations, the malleable length can be about 1%, 2%, 5%, 10%, 20%, 25%, 50% or more of the total length of the elongate body 360. In some implementations, the catheter advancement element 300 can have a working length of about 140 cm to about 143 cm and the elongate body 360 can have an insert length of about 49 cm. The insert length can be the PEBAX portion of the elongate body 360 that is about 49.5 cm. As such, the malleable length of the elongate body 360 can be between about 0.5 cm to about 25 cm or more. The shape change can be a function of a user manually shaping the malleable length prior to insertion or the distal end region 346 can be pre-shaped at the time of manufacturing into a particular angle or curve. Alternatively, the shape change can be a reversible and actuatable shape change such that the distal end region 346 forms the shape upon activation by a user such that the distal end region 346 can be used in a straight format until a shape change is desired by the user. The catheter advancement element 300 can also include a forming mandrel extending through the lumen of the elongate body 360 such that a physician at the time of use can mold the distal end region 346 into a desired shape. As such, the moldable distal end region 346 can be incorporated onto an elongate body 360 that has a guidewire lumen.

The elongate body 360 can extend along the entire length of the catheter 200, including the distal luminal portion 222 and the proximal control element 230 or the elongate body 360 can incorporate the proximal portion 366 that aligns generally side-by-side with the proximal control element 230 of the catheter 200. The proximal portion 366 of the elongate body 360 can be positioned co-axial with or eccentric to the elongate body 360. The proximal portion 366 of the elongate body 360 can have a lumen extending through it. Alternatively, the portion 366 can be a solid rod or ribbon having no lumen.

Again with respect to FIGS. 7A-7B and 8A-8B, like the distal luminal portion 222 of the catheter 200, the elongate body 360 can have one or more radiopaque markers 344 along its length. The one or more markers 344 can vary in size, shape, and location. One or more markers 344 can be incorporated along one or more parts of the catheter advancement element 300, such as a tip-to-tip marker, a tip-to-taper marker, an RHV proximity marker, a Fluoro-saver marker, or other markers providing various information regarding the relative position of the catheter advancement element 300 and its components. The at least one radiopaque marker can identify the tapered end region of the elongate body 360. In some implementations and as best shown in FIGS. 8A-8B, a distal end region can have a first radiopaque marker 344a and a second radiopaque marker 344b can be located to indicate the border between the tapering of the distal end region 346 and the more proximal region of the elongate body 360 having a uniform or maximum outer diameter. This provides a user with information regarding an optimal extension of the distal end region 346 relative to the distal end of the luminal portion 222 to minimize the lip at this distal end of the luminal portion 222 for advancement through tortuous anatomy. In other implementations, for example where the distal end region 346 is not necessarily tapered, but instead has a change in overall flexibility along its length, the second radiopaque marker 344b can be located to indicate the region where the relative flexibilities of the elongate body 360 (or the distal end region 346 of the elongate body 360) and the distal end of the luminal portion 222 are substantially the same. The marker material may be a platinum/iridium band, a tungsten, platinum, or tantalum-impregnated polymer, a metallic coil or braid, or another radiopaque marker. The radiopaque marker(s) preferably do not impact the flexibility of the distal end region 346 and elongate body 360. In some implementations, the radiopaque markers are extruded PEBAX loaded with tungsten for radiopacity. In some implementations, the proximal marker band can be about 2.0 mm wide, and the distal marker band can be about 2.5 mm wide to provide discernable information about the distal end region 346. In other implementations, the proximal marker is a different construction and/or material from the proximal marker. Additionally, the radiopaque marker bands 344a, 344b can be visible to a user without fluoroscopy, for example, prior to inserting the catheter system into the patient. The marker bands 344a, 344b can form a contrasting color visible to a user compared to a color of the polymer of the flexible elongate body, such as a black band relative to a white color of the polymer. The marker bands 344a, 344b can be useful in achieving a particular relative extension of the catheter advancement element 300 to the catheter 200 prior to insertion of the devices into an RHV.

The catheter 200 and catheter advancement element 300 (with or without a guidewire) can be advanced as a single unit through both turns of the carotid siphon. Both turns can be traversed in a single smooth pass or throw to a target in a cerebral vessel without the step-wise adjustment of their relative extensions and without relying on the conventional step-wise advancement technique with conventional microcatheters. The catheter 200 having the catheter advancement element 300 extending through it allows a user to advance them in unison in the same relative position from the first bend of the siphon through the second bend beyond the terminal cavernous carotid artery into the ACA and MCA. Importantly, the advancement of the two components can be performed in a single smooth movement through both bends without any change of hand position.

The catheter advancement element 300 can be juxtapositioned relative to the catheter 200 that provides an optimum relative extension between the two components for single smooth advancement. The catheter advancement element 300 can be positioned through the lumen of the catheter 200 such that its distal end region 346 extends just beyond a distal-most end 215 of the catheter 200. The distal end region 346 of the catheter advancement element 300 eliminates the stepped transition between the inner member and the outer catheter 200 thereby avoiding issues with catching on branching vessels within the region of the vasculature such that the catheter 200 may easily traverse the multiple angulated turns of the carotid siphon. The optimum relative extension, for example, can be the distal end region 346 of the elongate body 360 extending just distal to a distal-most end 215 of the catheter 200. A length of the distal end region 346 extending distal to the distal-most end 215 of the catheter 200 during advancement can be between 0.5 cm and about 4 cm. This juxtaposition can be a locked engagement with a mechanical element or simply by a user holding the two components together. The mechanical locking element can be a fixed or removable mechanical element 605 configured to connect to one or more of the catheter 200, the catheter advancement element 300, and the guidewire 500. The mechanical locking element 605 can be slidable along at least a length of the system components when coupled so that the mechanical attachment is adjustable. The mechanical locking element 605 can be a disposable feature or reusable for connecting to at least a portion of the shaft or a more proximal portion of the component, such as the luer or hub at a proximal end of the component. In some implementations, the mechanical locking element 605 can be clamped onto the catheter 200 and the catheter advancement element 300 in a desired relative position so that the two can be advanced together without the relative position being inadvertently changed. The relative position can be changed, if desired, while the mechanical locking element 605 is clamped onto the catheter 200 and the catheter advancement element 300. The mechanical locking element 605 can be additionally clamped onto a region of the guidewire 500 extending through the catheter advancement element 300 such that the relative position of all three components can be maintained during advancement until a relative sliding motion is desired. In still further implementations, the clamping position of the mechanical locking element 605 can be changed from engaging with a first combination of components (e.g., the catheter, catheter advancement element, and the guidewire) to a different combination of components (e.g., the catheter advancement element and the guidewire) depending on what phase of the method is being performed. In still further implementations, the guidewire 500 is held fixed relative to the catheter advancement element 300 via a rotating hemostatic valve coupled to the proximal hub 434 and the catheter advancement element 300 is held fixed to the catheter 200 by a separate mechanical locking element 605. Whether the relative position of the components is fixed by a mechanical element, a combination of mechanical elements, or by a user, the proximal portions 264 of each of the catheter 200 and the catheter advancement element 300 (and the guidewire 500, if present) are configured to be held at a single point by a user. For example, where the catheter and catheter advancement element are advanced and/or withdrawn manually, the single point can be between just a forefinger and thumb of the user.

The components can be advanced together with a guidewire, over a guidewire pre-positioned, or without any guidewire at all. In some implementations, the guidewire can be pre-assembled with the catheter advancement element 300 and catheter 200 such that the guidewire extends through a lumen of the catheter advancement element 300, which is loaded through a lumen of the catheter 200, all prior to insertion into the patient. The pre-assembled components can be simultaneously inserted into the sheath 400 and advanced together up through and past the turns of the carotid siphon. A guidewire may be located within the lumen 368 of the catheter advancement element 300 and parked proximal of the tapered distal end region 346 or proximal of the distal tip for potential use in the event the catheter advancement element without a guidewire does not reach the target location. For example, a distal tip of the guidewire 500 can be positioned about 5 cm to about 40 cm, or about 20 cm to about 30 cm proximal of the distal end region 346 of the catheter advancement element 300. At this location the guidewire does not interfere with the performance or function of the catheter advancement element. The guidewire can be positioned within the lumen of the catheter advancement element such that the distal end of the guidewire is within the catheter advancement element during the step of advancing the assembled system of devices together and is extendable from the catheter advancement element out the distal opening 326 when needed for navigation. In one example, a rescue guidewire is parked within the lumen of the catheter advancement element with a distal end of the guidewire about 0 cm to about 40 cm proximal or about 5 cm to about 35 cm proximal or about 7 cm to about 30 cm of the distal end of the catheter advancement element, preferably about 10 cm proximal of the distal end of the catheter advancement element. The guidewire at this parked position can provide additional support for the proximal portion of the system without affecting the flexibility and performance of the distal portion of the system.

Standard neurovascular intervention, and nearly all endovascular intervention, is predicated on the concept that a guidewire leads a catheter to a target location. The guidewires are typically pre-shaped and often find side-branches of off-target locations where the guidewire will bunch or prolapse causing time-consuming nuisances during interventions that often require repeated redirection of the guidewire by the operator to overcome. In addition, this propensity of a guidewire to enter side-branches can be dangerous. Guidewires are typically 0.014″ to 0.018″ (0.356 mm-0.457 mm) in the neuroanatomy and will find and often traumatize dissection flaps or small branches that accommodate this size, which can lead to small bleeds or dissections and further occlusion. In a sensitive area like the brain these events can be catastrophic. The tendency of a guidewire to bunch and prolapse can also cause a leading edge to the guidewire that can be advanced on its own or as part of a tri-axial system to create dissection planes and traumatize small vessels.

In contrast, the catheter advancement element 300 described herein preferentially stays in the larger lumen of a conduit vessel. The catheter advancement element 300 delivers to the largest lumen within the anatomy even in light of the highly tortuous anatomy and curves being navigated. The catheter advancement element 300 can preferentially take the larger lumen at a bifurcation or dissection flap while also following the current of the greatest blood flow thereby maintaining the general direction and angulations of the parent vessel. In viewing the standard anatomy found in the cerebral vasculature, the Circle of Willis is fed by two vertebral and two carotid conduit arteries. As these four arteries are the access points to the cerebral anatomy-the course of the catheter advancement element 300 can be identified and has been validated in standard cerebral anatomy models.

In the anterior circulation where the conduit artery point of entry for cerebral endovascular procedures is the internal carotid artery (ICA), the catheter advancement element can guide the large-bore catheter to the M1 segment of the middle cerebral artery (MCA) bypassing the anterior communicating artery (ACA) and anterior temporal branch (ATB). The very flexible nature of the catheter advancement element 300 combined with the distal flexible nature of most cerebral catheters combine to allow delivery through severe tortuosity. Independent of the tortuous nature of the course of the arteries, the catheter advancement element 300 tends to navigate the turns and deliver to the largest offspring from a parent artery, for example, ICA to M1 segment of the MCA. The M2 level branching of the MI can be variable, but is often seen to have two major M2 branches (superior and inferior) and, depending on the anatomy, which can vary significantly between patients, may be seen to bifurcate “equally” or “unequally.” If the caliber of the M2 branching is of similar size and angulation, the catheter advancement element 300 may take one of the two branches. If the target for catheter placement is not in a favorable angulation or size of artery, the catheter advancement element 300 may be curved (e.g., via shaping of a malleable distal tip) and directed or a guidewire may be used.

In some anatomies where the M2 bifurcation is “even” in size, a back-and-forth motion may aid in selecting one branch then the other while still avoid the need or use of a guidewire or a curved distal tip of the catheter advancement element. The back-and-forth motion can allow for the catheter advancement element to be directed into either branch of the M2. The catheter advancement element, even when initially straight, achieves some curvature that aids in directing it into a branch vessel. Thus, when an operator encounters an M2 bifurcation and there is a desire to cannulate either branch of an evenly divided bifurcation, selection of either branch is possible using the catheter advancement element without a guidewire.

Thus, main channels, such as the ICA, the middle cerebral artery and its tributaries in the anterior circulation will naturally be the pathway of preference for the described catheter advancement element and subsequence large-bore catheter delivery (via access from the ICA). A similar phenomenon can occur in the posterior circulation, which is accessed via the vertebral arteries arising from the subclavian arteries on the right and the left. The catheter advancement element will take the main channels in this circulation as well by traversing the vertebral arteries to the basilar artery and to the major tributaries of the basilar: the posterior cerebral artery and superior cerebellar arteries in the posterior circulation.

Navigation using the catheter advancement element can provide maximal deliverability with minimal vascular trauma. Catheters can cause “razoring” effects in a curved vessel because the blunt end of a large bore catheter can tend to take the greater curve in rounding a vessel when pushed by the operator. This blunt end can gouge or “razor” the greater curve with its sharp edge increasing the risk for dissection along an anatomic plane within the multilayered mid-or large-sized artery or vein (sec, e.g., Catheter Cardiovasc. Interv. 2014 February; 83(2):211-20). The catheter advancement element can serve to minimize the edge of these catheters. Positioning the catheter advancement element within the lumen of the large-bore catheter such that the taper marker of the catheter advancement element is aligned optimally with the distal tip marker of the catheter minimizes the edge and thereby eliminates “razoring” as the large-bore catheter is advanced through turns of the vessel. This is particularly useful for the cerebral anatomy. Treatments distal to the carotid siphon, particularly distal to the ophthalmic artery takeoff from the greater curve of the severe tortuosity of the final turn of the carotid siphon “S-turn”, the “anterior genu” of the carotid siphon typically seen as part of the terminal internal carotid artery (ICA) can be improved using the access system described herein. The specifics of the catheter advancement element in proper alignment within the large bore catheter (the “tip-to-taper” position noted by the distal tip marker) relative to the taper marker of the catheter advancement element maximize the likelihood that razoring and hang-up on the ophthalmic artery are avoided during manual advancement of the catheter system. The taper marker of the catheter advancement element can be positioned at or past the take-off of the ophthalmic artery to minimize these deleterious effects and allows the large-bore catheter to pass the ophthalmic artery without incident. In a relatively straight segment, which is common after passing the siphon, the large-bore catheter can be advanced over the catheter advancement element, which serves still as a guiding element to the target. The transition between the catheter advancement element and the distal edge of the large-bore catheter is insignificant, especially compared to the step changes present with a typical microcatheter or guidewire, which do not prevent hang-ups on branches, such as the ophthalmic artery. The catheter advancement element allows for maneuvering of the large-bore catheter to distal sites without use of a microcatheter or guidewire.

The systems described herein can but need not incorporate a guidewire. And, if a guidewire is used, it need not be advanced independently (i.e., unsheathed) to the target treatment site. Thus, the systems described herein can incorporate relatively large bore catheters that are delivered without disturbing anatomy with a guidewire, reducing the risk for stroke and downstream effects from fragmentation of an occlusion, and having improved efficiency. Additionally, the systems described herein are single-operator systems allowing the operator to work at a single RHV and, in the case of spined components, can manipulate all the elements being used to navigate the anatomy with single-handed “pinches.” This can be referred to as “monopoint.”

Any implant described herein may be used with any delivery system including but not limited to those described here, and may be delivered via access catheters including but not limited to those described here.

### Methods of Use

One or more components of the catheter systems, implants, and implant delivery systems described herein can be used to access and/or treat intracranial and cerebral diseases, such as delivery of expandable implants, such as stents or flow diverters, to treat intracranial and cerebral vessel narrowing, vasospasm, atherosclerotic disease, intracranial stenosis or other blockages in arteries, veins, or venous sinuses, aneurysms, and the like. The catheter systems described herein, including the implant delivery system 800, can be used to deliver expandable endovascular devices to a treatment site within the arterial system or within a venous system, for example, intracranial arteries or intracranial veins or dural venous sinuses. The catheter systems can provide “monopoint” manipulation at the base sheath for the various tools used in the method providing improved safety, case of use, and single operator manipulations compared to conventional systems. These catheter systems provide easy and quick access to target sites even through tortuous anatomy to reach the target lesion. The implants and implant delivery systems described herein provide improved, more accurate, and safer treatment of diseases. In addition, the implants described here have potentially reduced complication rates due to the geometry of the device apposition against the vessel wall.

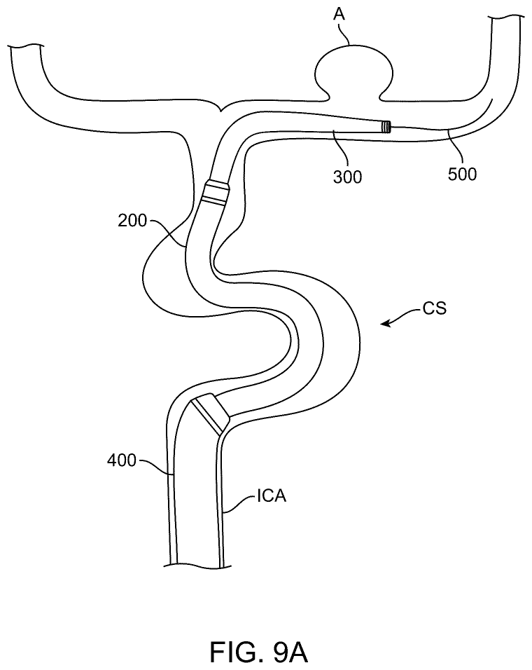

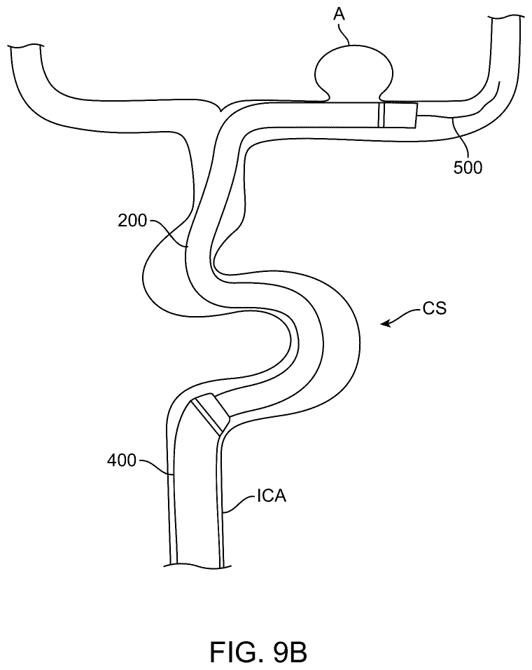

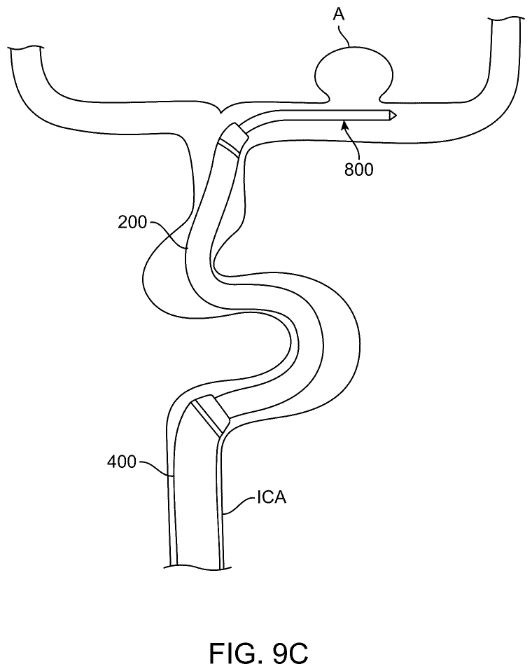

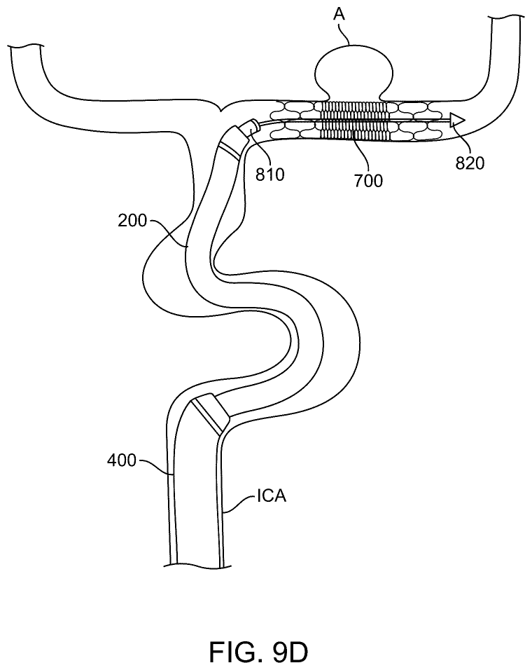

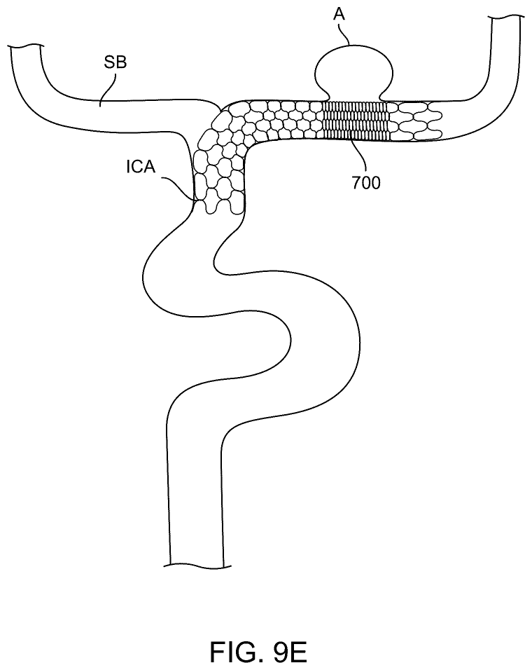

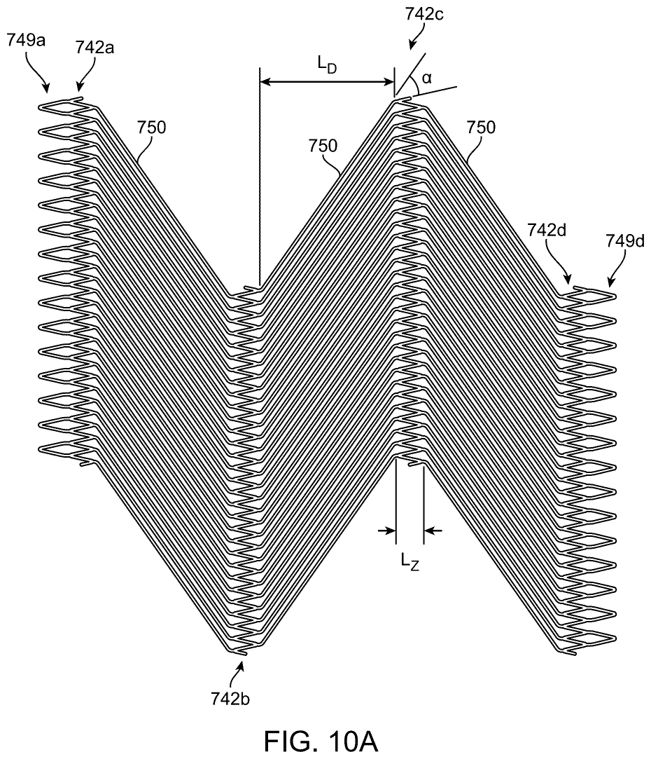

A method for the treatment of cerebral or intracranial aneurysm is now described. The method can include a flow diverter delivered using an implant delivery system 800 advanced over a guidewire (or advanced alone without any guidewire or advanced with a guidewire parked within a lumen of the delivery system) through an outer catheter extending through a base sheath. The outer catheter can be a conventional full-length catheter, but is preferably a catheter having a larger diameter distal luminal portion 222 coupled to a smaller diameter proximal control element 230 as shown in FIGS. 7A-7B so that monopoint manipulation at the base sheath hub is possible. The base sheath 400 can be introduced into a blood vessel at a vascular access point (e.g., femoral artery, radial, ulnar, or brachial arteries, or direct puncture of the carotid artery) and advanced to the level of at least the common carotid artery towards an intracranial or cerebral vessel having a segment with an aneurysm. An outer catheter 200 is advanced through the hub (e.g., an RHV 434) on the base sheath 400 until the distal end of the catheter 200 exits the distal opening 408 of the base sheath 400 (see FIG. 9A). The catheter 200 can be advanced to the site A intended for treatment, e.g. the site of an aneurysm. The outer catheter 200 can be part of a catheter system including an inner catheter 300 (also referred to herein as a catheter advancement element) having a tapered end region 346 that extends distal to the distal end of the outer catheter 200 as shown in FIGS. 8A and 8B. The outer catheter 200 can be navigated through the carotid siphon CS towards the treatment site A aided by the inner catheter 300. The outer catheter 200 and inner catheter 300 can be advanced together through the carotid siphon until at least a portion of the tapered end region 346 of the inner catheter 300 is positioned across the target treatment site A, e.g., the aneurysm as illustrated in FIG. 9A. Alternatively, a guidewire 500 can be advanced through the hub on the base sheath 400 and advanced until the guidewire 500 is positioned across the treatment site A while the outer catheter 200 remains parked at a location between the distal end of the base sheath 400 and the treatment site A (e.g., at or near the carotid siphon CS).

The distal end region of the outer catheter 200 can be advanced over or together with the inner catheter 300 and positioned across the treatment site A. The inner catheter 300 can be withdrawn from the outer catheter 200 and the outer catheter 200 maintained in position across the treatment site A (see FIG. 9B). The outer catheter 200 can have an inner diameter (ID) of between 2.0 mm and 3.0 mm that is configured to receive an implant 700, such as a flow diverter, mounted within an implant delivery system 800. The implant delivery system 800 and implant 700 can be advanced (e.g., through the hub of the outer catheter 200 or the hub of the base sheath 400 and into the distal tubular portion 222 of the catheter 200 if the catheter 200 is a partial length catheter) to the distal end region of the outer catheter 200. The outer catheter 200 can be withdrawn to expose the implant delivery system 800 while the implant delivery system 800 is maintained across the treatment site A (i.e., aneurysm in FIG. 9C). The implant 700 of the implant delivery system 800 can then be deployed across the treatment site A (see FIG. 9D).

The implant delivery system 800 can include an inner core member 820 and an outer restraining sleeve 810. The implant 700 can be mounted on the inner core member 820 and constrained by the outer restraining sleeve 810 during delivery. The implant 700 constrained by the outer restraining sleeve 810 can be deliverable through a delivery catheter having an inner diameter that is between 2.0 mm and 3.0 mm. Deployment of the implant 700 across the treatment site A can be achieved, for example, in reference to FIG. 9D, by retracting the outer restraining sleeve 810 of delivery system 800 to expose the implant 700 while the inner core member 820 remains in place distal to the treatment site A.

In another example, the implant 700 is an asymmetrical flow diverter with a longer proximal end. As shown in FIG. 9E, the treatment site A is an aneurysm very close to side branch SB. In this case, the implant 700 is a flow diverter that is positioned such that the dense middle portion is across the aneurysm, but the less dense proximal portion is long enough to cross the side-branch and be anchored firmly into the ICA. The less dense section allows flow into the side branch SB and if desired, additional interventional devices to access side branch SB.

The implant 700 can be any of the flow diverters described previously, such as shown in FIGS. 1A-1B, 2A-2B, 3, 10A-10B, or 11A-11D. For example, the implant can be a laser-cut expandable metal tube flow diverter. The flow diverter can be formed of first and second expandable tubes where each is a laser cut metal tube. The first expandable tube can be a laser cut metal tube and the second expandable tube can be a braided tube. Alternatively, the first expandable tube can be a laser cut metal tube and the second expandable tube can be a polymer sleeve. The flow diverter can have a compound construction. The compound construction can include two end sections constructed from laser-cut tube and a middle section that is a braid.

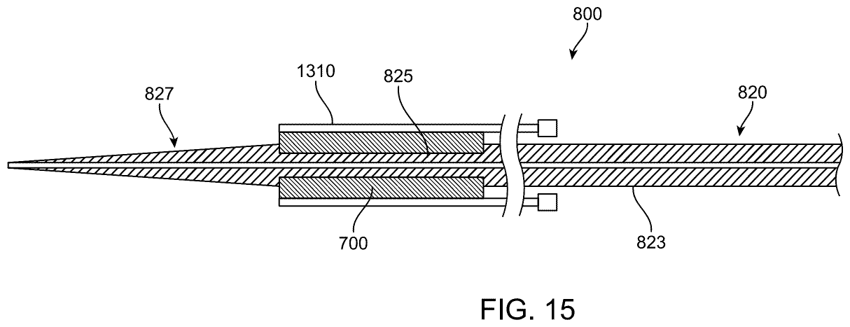

In an interrelated method of use, the implant delivery system 800 includes the inner core member 820 and does not have its own separate restraining sleeve. Instead, the catheter 200 of FIG, 7A serves as the outer restraining sleeve 810. As illustrated in FIG. 15, the implant 700 is pre-assembled on inner core member 820 and constrained on the recessed section 825 just proximal of the distal tip region 827. The outer catheter 200 is first advanced to be positioned across the treatment site A (e.g., aneurysm in the case of a flow diverter or a blood vessel narrowing in the case of a stent or other scaffolding device), for example, using an inner catheter 300 over a guidewire. The inner catheter 300 and optionally the guidewire are then removed, leaving the outer catheter 200 in place. The implant 700 mounted on the inner core member 820 of the implant delivery system 800 are then inserted into the outer catheter 200. An introducer component 1310 is optionally incorporated with the implant delivery system 800 to keep the implant 700 in a constrained diameter during this insertion into the catheter 200. Once the implant 700 is entirely contained within the outer catheter 200, the implant 700 and inner core member 820 are advanced until the implant 700 is positioned within a region of the outer catheter 200 that is positioned across the treatment site A. The outer catheter 200 can be withdrawn to unsleeve and thereby deploy the implant 700 across the treatment site A.

In embodiments of implant that can be stretched or compressed, the step of deploying the implant may also comprise pushing or pulling on the outer sheath 810 and inner core member 820 to compress the implant across specific locations like the neck of the aneurysm where a greater density of material is desired. This step may be facilitated by radiopaque markers at the boundaries of the compressible sections, so that this step can be guided by fluoroscopy.

A method for the treatment of a venous sinus is now described. Cerebral veins empty into large venous sinuses (also known as dural venous sinuses). The venous sinuses of the brain are situated within the subarachnoid space that facilitate outflow of blood from the cerebral veins of the brain to the jugular vein in the neck and back into the heart. There are seven paired dural sinuses including the transverse, cavernous, petrosal, sphenoparietal, sigmoid, and basilar sinuses. There are five unpaired dural sinuses including sagittal, straight, occipital, and intercavernous sinuses.

Blockages can occur in these sinuses to impair venous outflow from the brain. For example, cerebral venous sinus thrombosis (CVST) occurs when a blood clot forms in the brain's venous sinuses. Thrombosis within the venous sinuses builds pressure in the vessels leading to swelling and hemorrhage in the brain. CVST typically affects the superior sagittal, transverse, and cavernous sinuses. Risk factors for CVST include thrombophilia, acquired prothrombotic states, such as infections such as SARS-COV-2 leading to COVID-19, otitis, sinusitis, and mastoiditis; acquired prothrombotic states, such as pregnancy, antiphospholipid syndrome, and the puerperium; chronic inflammatory conditions, such as Wegener granulomatosis and sarcoidoisis, as well as trauma such as head injury, dehydration, and neurosurgical procedures. Swelling and hemorrhage due to high pressure built in the venous sinuses of the brain can also be due to non-thrombotic pathologies. In another example, Intracranial venous sinus stenosis is thought to play a role in the development of idiopathic intracranial hypertension (IIH), which may lead to visual disability and blindness. Arachnoid granulations or fibrous septae can create intrinsic discreet obstructions called “intrinsic stenosis”.

Surgical treatments of venous stenosis and CVST may also be performed to control and relieve pressure, for example, by deploying a stent or shunt to improve flow through the veins and lower pressure. Other surgical treatments, such as deploying a catheter to the dural sinuses for direct catheter thrombolysis or mechanical thrombectomy can also be performed. These surgical treatments require direct catheter access to the venous sinuses.

The navigation of the venous system is particularly problematic and difficult. The dural venous sinuses are located within deep, tortuous anatomy in an anatomical location that has potential for life-threatening outcomes. The cerebral venous system is far more variable than the arterial system. The variation can be among individuals and also between the hemispheres of the same brain. For example, tributaries leading into the various dural venous sinuses can be found along a particular sinus in non-standard, unpredictable locations patient-to-patient. The venous sinuses are also variable due to the presence of trabecular or septations that can arise from the lateral or medial aspect of a sinus wall and transect the space. The dural venous sinuses also include structures in the inner wall called chordae willisii, which are thought to be flow-improving structures. These intraluminal bands (trabeculae, bridges, synechiae, septations) vary in structure. The dural venous sinuses can also incorporate arachnoid granulations or villi, which are microscopic herniations of the arachnoid membrane that can invaginate through the walls of the venous sinuses. Each of these characteristics increase the difficulty in locating and navigating to a target site, in the dural sinuses, for example the site of an occlusion or other treatment. Together with the risk of perforating the dural venous sinus wall, this difficulty in access has generally limited the effectiveness and acceptance of surgical treatments of the venous sinuses. Release of embolic material is also a particular risk in venous clots because the venous flow takes the embolic material back to the right side of the heart and then the lungs.

Venous sinus treatments can be made difficult due to the large size of the vessels in relation to the catheter being delivered. Dural venous sinuses occupy a significant volume within the cranial cavity. Unlike on the arterial side of the cerebral vasculature, the deeper more distal sites within the intracranial venous sinuses can be nearly as large as the veins leading out of the cranium. The superior sagittal sinus (SSS) is typically considered the largest of the dural venous sinuses and can have a lumen diameter of 7.3 mm-8.8 mm depending on measurement technique used, whereas the proximal transverse sinus can be 7.5 mm-9.4 mm in diameter, the mid sigmoid sinus can be about 7.3 mm-10 mm in diameter, the proximal sigmoid sinus can be 8.6 mm-10.5 mm in diameter (Boddu et al. PLOS One 2018; 13(6):e0196275). Thus, distal treatment sites such as the superior sagittal sinus may be as large as the veins traversed to reach it. Treating the superior sagittal sinus by aspiration embolectomy to remove thrombosis has not been an effective treatment due to conventional neurovascular catheters being small, prone to clogging, and unable to achieve significant improvement in flow while the risk of perforation is high.

Difficulties in access also occur due to the rigid environment of the dura mater in which the venous sinuses lie. Tracking access catheters or stent delivery or other treatment catheters in arteries and veins that are fixed in position is much more challenging than when the vessels are surrounded by relatively compliant tissue, such as musculature. Not only is catheter advancement more difficult, but the risk of vessel injury is greater. This is especially true for catheters that are larger in diameter and closer to the cross-sectional dimensions of the lumen.

The catheters, catheter systems, implant delivery systems, and methods described herein also allow for safely and quickly accessing and treating the dural venous sinuses. In particular, the catheter systems described herein allow delivery of large-bore devices and implants deep within the dural venous sinuses of the brain to restore blood outflow from the brain quickly and while minimizing the risk of perforation, either through delivery of a stent or aspiration of the blockage. Delivery of large bore access devices to the dural venous sinuses is also beneficial to the delivery of endovascular implants to target sites in the venous sinuses, for example the sagittal venous sinus.

The method of stenting a venous sinus can include using a stent delivered by an implant delivery system 800 that is advanced over a guidewire or advanced alone without any guidewire or advanced with a guidewire parked within a lumen of the delivery system. The implant delivery system 800 can be advanced through an outer catheter extending through a base sheath. The outer catheter can be a conventional full-length catheter, but is preferably a catheter having a larger diameter distal luminal portion 222 coupled to a smaller diameter proximal control element 230 as shown in FIGS. 7A-7B so that monopoint manipulation at the base sheath hub is possible.

The base sheath 400 can be introduced via direct puncture of the internal jugular vein and advanced to the level of at least the bulb. An outer catheter 200 is advanced through the hub (e.g., an RHV 434) on the base sheath 400 until the distal end of the catheter 200 exits the distal opening 408 of the base sheath 400. The catheter 200 can be advanced using a catheter advancement element 300 as described elsewhere herein, that has a tapered end region 346 that leads the outer catheter 200 as the catheter system navigates through the venous system. The outer catheter 200 and the inner catheter 300 can be advanced together as a single unit in the advancement configuration (i.e., the tapered end region 346 extending a preferred distance outside the distal end of the outer catheter 200) to the superior sagittal sinus. The inner catheter 300 can be removed from the lumen of the outer catheter 200, while the proximal end of the outer catheter 200 is held fixed by a user. An implant delivery system 800 can then be inserted through the lumen of the outer catheter 200 towards the distal end of the outer catheter 200 positioned at or near the target site. The implant delivery system 800 can be positioned relative to the target site as desired for deployment of the implant. The outer catheter 200 can be positioned at the treatment site such that retracting the outer catheter 200 exposes the implant delivery system 800 for deployment of the implant, such as a stent, at the treatment site. The implant delivery system 800 can deploy the implant 700 as described elsewhere herein by retracting the outer restraining sheath. In other implementations, the outer catheter 200 provides restraining function for the implant of the implant delivery system 800 such that retracting the outer catheter 200 releases the implant from constraining forces such that it is deployed at the treatment site.

### Materials

One or more components of the catheters, delivery systems, and implant described herein may include or be made from a variety of materials including one or more of a metal, metal alloy, polymer, a metal-polymer composite, ceramics, hydrophilic polymers, polyacrylamide, polyethers, polyamides, polyethylenes, polyurethanes, copolymers thereof, polyvinyl chloride (PVC), PEO, PEO-impregnated polyurethanes, such as Hydrothane, Tecophilic polyurethane, Tecothane, PEO soft segmented polyurethane blended with Tecoflex, thermoplastic starch, PVP, and combinations thereof, and the like, or other suitable materials.

Some examples of suitable cut-tube or flat metal material includes Nitinol, Layered tube with Nitinol on outside and inner core of radiopaque material, such as tantalum, platinum, iridium, gold, alloy etc. Additionally, material could be cobalt, cobalt alloy, or stainless steel.

Some examples of suitable metals and metal alloys include stainless steel, such as 304V, 304L, and 316LV stainless steel; mild steel; nickel-titanium alloy, such as linear-clastic and/or super-elastic Nitinol; other nickel alloys, such as nickel-chromium-molybdenum alloys (e.g., UNS: N06625, such as INCONEL® 625, UNS: N06022, such as HASTELLOY® C-22®, UNS: N10276, such as HASTELLOY® C276®, other HASTELLOY® alloys, and the like), nickel-copper alloys (e.g., UNS: N04400, such as MONEL® 400, NICKELVAC® 400, NICORROS® 400, and the like), nickel-cobalt-chromium-molybdenum alloys (e.g., UNS: R30035, such as MP35-N® and the like), nickel-molybdenum alloys (e.g., UNS: N10665, such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003,such as ELGILOY®, PHYNOX®, and the like); platinum enriched stainless steel; titanium; combinations thereof; and the like; or any other suitable material and as described elsewhere herein.

Inner liner materials of the catheters described herein can include low friction polymers, such as PTFE (polytetrafluoroethylene) or FEP (fluorinated ethylene propylene), PTFE with polyurethane layer (Tecoflex). Reinforcement layer materials of the catheters described herein can be incorporated to provide mechanical integrity for applying torque and/or to prevent flattening or kinking, such as metals including stainless steel, Nitinol, Nitinol braid, helical ribbon, helical wire, cut stainless steel, or the like, or stiff polymers, such as PEEK. Reinforcement fiber materials of the catheters described herein can include various high tenacity polymers like Kevlar, polyester, meta-para-aramide, PEEK, single fiber, multi-fiber bundles, high tensile strength polymers, metals, or alloys, and the like. Outer jacket materials of the catheters described herein can provide mechanical integrity and can be contracted of a variety of materials, such as polyethylene, polyurethane, PEBAX, nylon, Tecothane, and the like. Other coating materials of the catheters described herein include paralene, Teflon, silicone, polyimide-polytetrafluoroetheylene, and the like. The inner liner may further include different surface finishes, such as dimples, bumps, ridges, troughs. The surface finishes may be randomly disposed, linearly disposed, spirally disposed, or otherwise disposed using a specific pattern along the length of the catheter. It is further contemplated that the inner liner may include a mixture of different surface finishes, for example, one section may have dimples, another section may have troughs, etc. Additionally, the surface finish may be incorporated along the entire length of the catheter or only in sections of the catheter. It is also contemplated that the inner liner may further include an electrosprayed layer, whereby materials could be incorporated into the inner liner. Examples of materials can include low friction materials as described above. Alternatively, the electrosprayed or electrospun layer may incorporate a beneficial agent that becomes free from the coating when exposed to blood, or to compression from a clot, for example, the beneficial agent may be a tissue plasminogen activator (tPA), or heparin encased in alginate.

Implementations describe catheters and delivery systems and methods to deliver catheters to target anatomies. However, while some implementations are described with specific regard to delivering catheters to a target vessel of a neurovascular anatomy, such as a cerebral vessel, the implementations are not so limited and certain implementations may also be applicable to other uses. For example, the catheters can be adapted for delivery to different neuroanatomies, such as subclavian, vertebral, carotid vessels as well as to the coronary anatomy or peripheral vascular anatomy, to name only a few possible applications. It should also be appreciated that although the systems described herein are described as being useful for treating a particular condition or pathology, that the condition or pathology being treated may vary and are not intended to be limiting.

In an aspect, provided is a flow diverter including a self-expanding tubular member having a plurality of expandable cells, each of the expandable cells having interconnected struts and bridges. The tubular member has a constrained configuration having a first outer diameter of at least 1.0 mm sized for delivery using a flow diverter delivery system and an expanded configuration having a second outer diameter larger than the first outer diameter. The tubular member has one or more of a proximal end zone, a distal end zone, and a middle zone located between the proximal end zone and the distal end zone. In some embodiments, there is further a transition section between zones, for example a transition section between the distal end zone and the middle zone, and a transition section between the middle zone and the proximal end zone. In some embodiments, the distal end zone is a different length than the proximal end zone. In some embodiments, the tubular member has just a distal end zone or just a proximal end zone. In some embodiment, the tubular member has a single zone.

At least the middle zone of the tubular member is laser-cut to have a material coverage of at least 25% when the tubular member is in the expanded configuration. At least the distal and proximal end zones have a larger cell size with less material coverage, designed to open easily and minimize obstruction of perforators and side branches on either side of the aneurysm. In some embodiments one or both of the distal and proximal end section are flared to improve anchoring and apposition to the wall.

The pattern is a closed cell pattern, with a series of zig-zag circumferential rings connected by diagonal struts. The pattern may include variations along the length, with a dense middle section designed to be placed across the aneurysm, and then one or more less dense sections on either end. In one configuration, the transition from dense to less dense sections can be made by connecting adjacent zig-zags with a V-strut, to halve the number of peaks for the next section. This density reduction can occur one or more times along the end sections of the flow diverter.

The middle zone can have properties different from one or both of the proximal end zone and distal end zone. The middle zone can have greater material coverage than one or both of the proximal end zone and the distal end zone. One or both of the proximal end zone and the distal end zone can be laser-cut to have a material coverage that is less than the material coverage of the middle zone. The material coverage of the middle zone can be between 25%-35% when the tubular member is in the expanded configuration and the proximal and distal end zones can have a material coverage less than the material coverage of the middle zone. At least one of the proximal end zone, the middle zone, and the distal end zone can include at least one radiopaque marker. A length of the flow diverter in the constrained configuration can be less than 1% different from a length of the flow diverter in the expanded configuration. A length of the flow diverter in the constrained configuration can be less than about 5% different from a length of the flow diverter in the expanded configuration. A length of the flow diverter in the constrained configuration can be less than about 10% different from a length of the flow diverter in the expanded configuration.

In some embodiments, the tubular member end sections include features for attaching radiopaque components to serve as RO markers to aid in locating the device under fluoroscopy.

In an interrelated aspect, provided is a method of treating intracranial or cerebral aneurysm including advancing a catheter system through a base sheath towards an intracranial or cerebral vessel having a segment with an aneurysm. The catheter system includes an inner catheter having a tubular elongate body with a single lumen and a flexible, distal tapered end region; and an outer catheter having a catheter lumen and a distal end. The method includes positioning the tapered end region of the inner catheter distal to the distal end of the outer catheter; crossing the segment of vessel with the aneurysm with at least a portion of the tapered end region of the inner catheter; advancing the outer catheter over the inner catheter and positioning a distal end region of the outer catheter across the lesion; withdrawing the inner catheter from the catheter lumen and maintaining the outer catheter in place across the aneurysm; advancing a flow diverter delivery system comprising a flow diverter through the catheter lumen to the distal end region of the outer catheter; withdrawing the outer catheter while maintaining the flow diverter delivery system in place; and deploying the flow diverter across the segment with the aneurysm.

In an interrelated aspect, provided is a method of performing a medical procedure at a treatment site in a brain of a patient including positioning a system of devices into an advancement configuration. The system of devices includes a catheter having a catheter lumen, an inner diameter, and a distal end; and an inner member sized and shaped to slide within the catheter lumen. The inner member defines a single lumen and has a distal portion. The distal portion has a first outer diameter that tapers distally to a second outer diameter that is smaller than the first outer diameter, and the inner member transitions in flexibility from a proximal end of the inner member to a distal end of the inner member, the distal end of the inner member being more flexible than the distal end of the catheter. When positioned in an advancement configuration, the inner member extends coaxially through the catheter lumen until the distal portion of the inner member is positioned distal to the distal end of the catheter. The method includes advancing the catheter and the flexible inner member to a target location to an access point of entry while the system of devices is positioned in the advancement configuration; positioning the catheter at the treatment site, the treatment site comprising an aneurysm; removing the inner member from the patient; and treating the aneurysm through the catheter. The step of treating can include delivering a flow diverter to the aneurysm through the catheter.

In an interrelated aspect, provided is a flow diverter delivery system including a flow diverter having a tubular structure and configured to treat an aneurysm in an intracranial vessel, the flow diverter having a constrained configuration having a first outer diameter and an expanded configuration having a second outer diameter; an inner core member; and an outer restraining sleeve. During delivery of the device, the flow diverter is positioned on a distal section of the inner core member and maintained in the constrained configuration by the outer restraining sleeve.

The inner core member may include features which maintain the position of the flow diverter during advancement of the delivery system to the target site, and during deployment of the flow diverter at the site. For example, the inner member is an elongate shaft with a recessed section near a distal end region of the elongate shaft, the recessed section sized to receive the tubular structure of the flow diverter when the flow diverter is in the constrained configuration. The proximal end of the recessed section steps up in outer diameter, to a dimension at or larger than the inner diameter of the flow diverter, and thus serves as a backstop to prevent proximal movement of the flow diverter during deployment. In an embodiment, a secondary component such as a washer or short tube segment constructed out of harder material than the inner core member, e.g., PEEK or metal, can be added to the proximal end of the recessed section to act as a firmer back-stop to the flow diverter.

Alternately, the inner member may comprise two components, an inner component and a mid-component. The two components are axially fixed with respect to each other. The inner component is sized to receive the tubular structure of the flow diverter when the flow diverter is in the constrained configuration. The mid-component terminates proximal to the inner component and has an outer diameter the same or larger size than the inner diameter of the flow diverter, and thus serves as a backstop to the flow diverter. The mid-component may also include features on its distal end which couple with features on the flow diverter to “capture” the flow diverter when in the constrained configuration, thus enabling re-sheathing and repositioning of the flow diverter during deployment. In some embodiments, the features on the flow diverter which are used to “capture” the flow diverter also serve as the radiopaque markers on the proximal end of the device.

The inner member may also include an atraumatic distal tip region located distal to the region where the flow diverter is housed. The distal tip region has a taper from a first outer diameter of the elongate shaft to a second outer diameter of the elongate shaft.

The system includes an outer restraining sleeve having an inner diameter sized to receive the inner core member and the flow diverter in the constrained configuration. The outer restraining sleeve is retractable at least a distance to deploy the flow diverter.

The inner diameter of the restraining sleeve can be size-matched to the first outer diameter of the elongate shaft to reduce an annular space at a leading end of the flow diverter delivery system. The distal tip region can include at least one radiopaque marker at a distal end. The distal tip region can include a second radiopaque marker positioned to identify the taper.

In an interrelated aspect, provided is a flow diverter having a self-expanding tubular member having a proximal end, a distal end, and a longitudinal axis. The tubular member has a constrained configuration with a first outer diameter sized for delivery and an expanded configuration having a second outer diameter larger than the first outer diameter. The tubular member includes a plurality of expandable cells, each cell having interconnected struts and bridges arranged in circumferential rings. The circumferential rings form rows of the expandable cells extending between the proximal and distal ends of the tubular member parallel with the longitudinal axis. The tubular member has a proximal end zone near the proximal end of the tubular member, a distal end zone near the distal end of the tubular member, and a middle zone located between the proximal end zone and the distal end zone. At least the distal end zone includes at least one rail formed of bridges interconnecting each circumferential ring of expandable cells within a single row.

The at least one rail can enable re-sheathing of the distal end zone in a delivery system after at least partial deployment of the distal end zone from the delivery system. At least the middle zone of the tubular member can be laser-cut to have a material coverage of at least 25% when the tubular member is in the expanded configuration.

In an interrelated aspect, provided is a flow diverter configured to expand from a constrained state to an expanded state. The flow diverter includes a first tube of superelastic material formed of a plurality of cells having a first material coverage; and a second tube of superelastic material formed of a plurality of cells having a second material coverage. The second tube is positioned inside of the first tube so that an overlap of the plurality of expandable cells of the first tube and the plurality of expandable cells of the second tube forms a third material coverage that is greater than the first material coverage and the second material coverage when the flow diverter is in the expanded state.

The second tube can be locked in position inside the first tube by a feature in a cut pattern of at least one of the first tube and the second tube. The feature can include a slot in the first or the second tube and tab configured to protrude into the slot to lock the first and second tubes together. The feature can include a hole in the first or the second tube and a malleable disk configured to insert within the hole to lock the first and second tubes together. At least one of the first tube and the second tube can be non-braided and laser-cut.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference, and are not intended to limit the use or orientation of the catheters and/or delivery systems to a specific configuration described in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value. One inch or 1″ corresponds to 2.54 cm (SI-units).

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The components of the systems disclosed herein may be packaged together in a single package or separately. The finished package would be sterilized using sterilization methods such as Ethylene oxide or radiation and labeled and boxed. Instructions for use may also be provided in-box or through an internet link printed on the label.

### P EMBODIMENTS

P Embodiment 1. A flow diverter comprising: a self-expanding tubular member comprising a plurality of expandable cells, each of the expandable cells comprising interconnected zig-zag rings and diagonal struts, wherein the tubular member has a constrained configuration having a first outer diameter of at least 1.0 mm sized for delivery using a flow diverter delivery system and an expanded configuration having a second outer diameter larger than the first outer diameter, and wherein the tubular member has a proximal end zone, a distal end zone, and a middle zone located between the proximal end zone and the distal end zone, wherein at least the middle zone of the tubular member is laser-cut to have a material coverage of at least 25% when the tubular member is in the expanded configuration.
P Embodiment 2. The flow diverter of P Embodiment 1, wherein the distal end zone and the proximal end zone have lower material coverage than the middle zone.
P Embodiment 3. The flow diverter of P Embodiment 2, wherein the proximal end zone is longer than the distal end zone.
P Embodiment 4. The flow diverter of P Embodiment 2, wherein the proximal end zone is shorter than the distal end zone.
P Embodiment 5. A method of treating intracranial or cerebral aneurysm, the method comprising: advancing a catheter system through a base sheath towards an intracranial or cerebral vessel having a segment with an aneurysm, the catheter system comprising: an inner catheter having a tubular elongate body with a single lumen and a flexible, distal tapered end region; and an outer catheter having a catheter lumen and a distal end; positioning the tapered end region of the inner catheter distal to the distal end of the outer catheter; crossing the segment of vessel with the aneurysm with at least a portion of the tapered end region of the inner catheter; advancing the outer catheter over the inner catheter and positioning a distal end region of the outer catheter across the aneurysm; withdrawing the inner catheter from the catheter lumen and maintaining the outer catheter in place across the aneurysm; advancing a flow diverter delivery system comprising a flow diverter through the catheter lumen to the distal end region of the outer catheter; withdrawing the outer catheter while maintaining the flow diverter delivery system in place; and deploying the flow diverter across the segment with the aneurysm.
P Embodiment 6. The method of P Embodiment 5, wherein the flow diverter has a longer proximal zone than distal zone, and wherein deploying the flow diverter across the segment with the aneurysm also comprises deploying the low-density proximal zone across a side branch.

1. A flow diverter comprising:
a self-expanding tubular member comprising a plurality of expandable cells, each of the expandable cells comprising interconnected zig-zag rings and diagonal struts,
wherein the tubular member has a constrained configuration having a first outer diameter of at least 1.0 mm sized for delivery using a flow diverter delivery system and an expanded configuration having a second outer diameter larger than the first outer diameter, and
wherein the tubular member has a proximal end zone, a distal end zone, and a middle zone located between the proximal end zone and the distal end zone, wherein at least the middle zone of the tubular member is laser-cut to have a material coverage of at least 25% when the tubular member is in the expanded configuration.

2. The flow diverter of claim 1, wherein the distal end zone and the proximal end zone have lower material coverage than the middle zone.

3. The flow diverter of claim 1, wherein the proximal end zone is longer than the distal end zone.

4. The flow diverter of claim 1, wherein the proximal end zone is shorter than the distal end zone.

5. A method of treating intracranial or cerebral aneurysm, the method comprising:
advancing a catheter system through a base sheath towards an intracranial or cerebral vessel having a segment with an aneurysm, the catheter system comprising:
an inner catheter having a tubular elongate body with a single lumen and a flexible, distal tapered end region; and
an outer catheter having a catheter lumen and a distal end;
positioning the tapered end region of the inner catheter distal to the distal end of the outer catheter;
crossing the segment of vessel with the aneurysm with at least a portion of the tapered end region of the inner catheter;
advancing the outer catheter over the inner catheter and positioning a distal end region of the outer catheter across the aneurysm;
withdrawing the inner catheter from the catheter lumen and maintaining the outer catheter in place across the aneurysm;
advancing a flow diverter delivery system comprising a flow diverter through the catheter lumen to the distal end region of the outer catheter;
withdrawing the outer catheter while maintaining the flow diverter delivery system in place; and
deploying the flow diverter across the segment with the aneurysm.

6. The method of claim 5, wherein the flow diverter has a longer proximal zone than distal zone, and wherein deploying the flow diverter across the segment with the aneurysm also comprises deploying the low-density proximal zone across a side branch.

7. A flow diverter comprising:
a self-expanding tubular member comprising a plurality of expandable cells, each of the expandable cells comprising interconnected zig-zag rings and diagonal struts,
wherein the tubular member has a constrained configuration having a first outer diameter sized for delivery using a flow diverter delivery system and an expanded configuration having a second outer diameter larger than the first outer diameter, and
wherein the tubular member has a proximal end zone near a proximal end, a distal end zone near a distal end, and a middle zone located between the proximal end zone and the distal end zone, wherein at least the middle zone of the tubular member is laser-cut to have a higher material coverage when the tubular member is in the expanded configuration compared to the proximal end zone or the distal end zone.

8. The flow diverter of claim 7, wherein each of the zig-zag rings has a length, a height, and a number, wherein each of the diagonal struts has a length and an angle, and wherein the tubular member has a longitudinal axis extending along a lumen of the flow diverter from the proximal end to the distal end.

9. The flow diverter of claim 8, wherein the height and the number of zig-zag rings and the length and the angle of the diagonal struts provide a targeted material coverage of at least 25% in the expanded configuration and a maximum outer diameter of no greater than 0.070″ in the collapsed configuration.

10. The flow diverter of claim 8, wherein each end of the diagonal struts where the diagonal struts connect with the zig-zag rings are substantially parallel to the longitudinal axis of the flow diverter while a mid-section of each diagonal strut is arranged at an angle to the longitudinal axis of the flow diverter.

11. The flow diverter of claim 10, wherein each diagonal strut is connected at a first end to a peak in a first zig-zag ring and is connected at a second end to a peak of an adjacent zig-zag ring.

12. The flow diverter of claim 11, wherein the second end connected to the peak of the adjacent zig-zag ring is rotated at least 4 peaks circumferentially from the first end connected to the peak in the first zig-zag ring.

13. The flow diverter of claim 12, wherein the diagonal struts connected to the first zig-zag ring are angled in a direction opposite from the diagonal struts connected to the adjacent zig-zag ring.

14. The flow diverter of claim 8, wherein the length of the diagonal struts located in the proximal end zone, the distal end zone, and the middle zone are substantially the same while the length of the zig-zag rings increases from the middle zone to the proximal zone and from the middle zone to the distal zone.

15. The flow diverter of claim 8, wherein the middle zone has three zig-zag rings and at least two sections of diagonal struts.

16.-24. (canceled)

25. The flow diverter of claim 15, wherein a distal region of the at least two sections of diagonal struts connects to a distal zig-zag ring and a proximal-most region of the at least two diagonal struts connects to a proximal zig-zag ring.

26. The flow diverter of claim 25, wherein the distal-most end of the flow diverter incorporates a terminal zig-zag ring distal to a distal-most zig-zag ring and the proximal-most end of the flow diverter incorporates a terminal zig-zag ring proximal to a proximal-most zig-zag ring.

27. The flow diverter of claim 26, wherein an amplitude of the terminal zig-zag ring distal to the distal-most zig-zag ring and an amplitude of the terminal zig-zag ring proximal to the proximal-most zig-zag ring is the same height as or a greater height than that of a respective adjacent zig-zag ring.

28. The flow diverter of claim 15, wherein the at least two sections of diagonal struts connect one V formed by a pair of adjacent angled struts of a first zig-zag ring to one V formed by another pair of adjacent angled struts of a second zig-zag ring.

29. The flow diverter of claim 15, wherein each diagonal strut of the at least two sections of diagonal struts has a distal end that connects to a first zig-zag ring and a proximal end that connects to an adjacent zig-zag ring, wherein a connection point between each diagonal strut and the zig-zag rings is at a peak, a valley, or mid-strut between the peak and the valley.

30. The flow diverter of claim 15, wherein a first diagonal strut connects a valley of a pair of angled struts in a first zig-zag ring to a connection point of an angled strut of an adjacent zig-zag ring.

31. The flow diverter of claim 30, wherein a portion of the first diagonal strut that is within the valley lies substantially parallel to the longitudinal axis of the flow diverter.

32. The flow diverter of claim 31, wherein the first diagonal strut curves in a first direction away from the longitudinal axis moving proximally from its distal end to extend substantially diagonal to the longitudinal axis.

33. The flow diverter of claim 32, wherein a proximal end of the first diagonal strut curves in an opposite direction to be parallel to the longitudinal axis of the flow diverter and to be positioned within a valley of an adjacent zig-zag ring.

34. The flow diverter of claim 33, wherein a first portion of a second diagonal strut connects between a peak and a valley of the pair of angled struts in the first zig-zag ring to a valley in the adjacent zig-zag ring.

35. The flow diverter of claim 34, wherein a third diagonal strut 750c connects at a peak of the pair of angled struts in the first zig-zag ring has a connection point between a peak and a valley of a second pair of angled struts in the second zig-zag ring.

36. The flow diverter of claim 35, wherein each of the first diagonal strut and the second diagonal strut incorporates a curve near the distal end of the diagonal struts and the proximal end of the diagonal struts so that at least a portion of each of the first diagonal strut and the second diagonal strut lies parallel to the longitudinal axis of flow diverter near its connection point with its respective angled strut.

37. The flow diverter of claim 36, wherein connections formed by the first diagonal strut, the second diagonal strut, the third diagonal strut, and a fourth diagonal strut between adjacent zig-zag rings creates a circumferential pattern of connections.

38. The flow diverter of claim 37, wherein the circumferential pattern of connections repeats around a circumference of and along the length of flow diverter within the middle zone.

39. The flow diverter of claim 37, wherein the circumferential pattern of connections comprises:
a proximally-facing valley of the first zig-zag ring connected to a mid-strut point of the second zig-zag ring;
a mid-strut point of the first zig-zag ring connected to distally-facing valley of the second zig-zag ring;
a proximally-facing peak of the first zig-zag ring connected to mid-strut point of the second zig-zag ring; and
a mid-strut point of the first zig-zag ring connected to distally-facing peak of the second zig-zag ring.

40. The flow diverter of claim 8, wherein each diagonal strut has a substantially identical length from a distal end connection with an angled strut of a first zig-zag ring to a proximal end connection with an angled strut of an adjacent zig-zag ring allowing the flow diverter to be constrained in a tubular shape without any of the diagonal struts being placed under tension.

41. The flow diverter of claim 40, wherein each diagonal strut has a curvature near the distal end connection and near the proximal end connection allowing for each diagonal strut to form the distal end connection and the proximal end connection with the angled struts in a substantially parallel manner relative to the longitudinal axis of the flow diverter.

42.-50. (canceled)

51. An implant delivery system comprising:
an inner core member comprising an elongate shaft having a single inner lumen, a recessed section, and a tip distal to the recessed section;
an outer tubular member having a length sufficient to extend over the recessed section and that is retractable relative to the inner core member to expose the recessed section; and
an expandable device,
wherein when the expandable device is assembled for delivery by the delivery system, the expandable device is mounted around the recessed section of the inner tubular member and the distal end of the outer tubular member is positioned distal to the expandable device to constrain the expandable device within the recessed section, and the tip projects distal to the distal end of the outer tubular member, the tip having a flexibility, a shape, a taper length, and a taper angle configured for atraumatic delivery of the delivery system to a vessel in the brain with or without a guidewire.

52. The implant delivery system of claim 51, wherein the inner core member comprises an outer diameter proximal to the tip that is sized to minimize a distal-facing lip of the outer tubular member while allowing for movement between the inner core member and the outer tubular member upon application of a relatively small load.

53.-54. (canceled)

55. The implant delivery system of claim 51, wherein the tip tapers along at least a portion of a length of the tip distally from the outer diameter to a smaller outer diameter near a distal-most end of the inner core member.

56.-68. (canceled)

69. The implant delivery system of claim 51, wherein the tip comprises 3-5 material transitions from a distal-most end of the elongate shaft to the recessed section.

70. The implant delivery system of claim 51, wherein the elongate shaft and the recessed section are constructed to maintain axial integrity during deployment of the expandable device.

71.-109. (canceled)

110. A system for treating an intracranial vessel, the system comprising:
an access catheter comprising a lumen extending from a proximal opening to a distal opening at a distal end of the access catheter; and
an implant delivery system sized to be received within the lumen of the access catheter and having no separate restraining sleeve, the implant delivery system comprising:
an inner core member comprising an elongate shaft having a single inner lumen, a recessed section, and a tip distal to the recessed section; and
an expandable device mounted around the recessed section,
wherein the access catheter functions as a restraining sleeve for the expandable device of the implant delivery system.

111.-159. (canceled)

160. The system of claim 110, wherein the access catheter comprises:
a flexible, distal luminal portion having the lumen extending from the proximal opening at a proximal end of the flexible, distal luminal portion and the distal opening at the distal end; and
a proximal tether element extending proximally from a point of attachment near the proximal end of the flexible distal luminal portion, wherein an outer diameter of a portion of the proximal tether element near the point of attachment is smaller than an outer diameter of the distal luminal portion near the point of attachment.

161. A method of using the system of claim 160, comprising:
positioning the access catheter over a treatment site within the intracranial vessel;
inserting the implant delivery system into the lumen of the access catheter using the introducer component;
advancing the implant delivery system through the lumen towards the treatment site; and
unsleeving the expandable device mounted around the recessed section by withdrawing proximally the access catheter from over the expandable device to deploy the expandable device at the treatment site.

162.-180. (canceled)
